(19) United States
(12) Patent Application Publication
(10) Pub. No.: US 2025/0368629 A1
(43) Pub. Date: Dec. 4, 2025

(54) FLAVONOID COMPOUNDS AND METHODS AND MATERIALS FOR USING FLAVONOID COMPOUNDS TO TREAT FIBROTIC CONDITIONS

(71) Applicant: Mayo Foundation for Medical Education and Research, Rochester, MN (US)

(72) Inventors: Andrew J. Haak, Rochester, MN (US); Steven P. O'Hara, Rochester, MN (US); Maria E. Guicciardi, Rochester, MN (US); Petra Hirsova, Rochester, MN (US); Nathan K. LeBrasseur, Rochester, MN (US); Marissa J. Schafer, Rochester, MN (US)

(21) Appl. No.: 18/874,799

(22) PCT Filed: Jun. 13, 2023
(86) PCT No.: PCT/US2023/025221

§ 371 Date: Dec. 13, 2024

Related U.S. Application Data
(60) Provisional application No. 63/351,485, filed on Jun. 13, 2022.

(51) Int. Cl. C07D 405/04; A61K 31/352; A61P 29/00; C07D 311/62; C07D 405/12; C07D 407/12; C07D 409/12

(52) U.S. Cl. CPC C07D 405/04; A61K 31/352; A61P 29/00; C07D 311/62; C07D 405/12; C07D 407/12; C07D 409/12

(57) ABSTRACT

This document relates to flavonoid compounds and methods and materials for using flavonoid compounds to treat one or more fibrotic conditions (e.g., idiopathic pulmonary fibrosis (IPF), non-alcoholic steatohepatitis (NASH), primary sclerosing cholangitis (PSC), and/or ocular fibrosis). For example, one or more flavonoid compounds having the structure of Formula (I) or Formula (II) can be administered to a mammal (e.g., a human) having one or more fibrotic conditions (e.g., IPF, NASH, PSC, and ocular fibrosis) to treat the mammal.

### CROSS-REFERENCE TO RELATED APPLICATIONS

This application claims the benefit of U.S. Patent Application Ser. No. 63/351,485, filed on Jun. 13, 2022. The disclosure of the prior application is considered part of, and is incorporated by reference in, the disclosure of this application.

### TECHNICAL FIELD

This document relates to flavonoid compounds and methods and materials for using flavonoid compounds to treat fibrotic conditions.

### BACKGROUND INFORMATION

Fibrotic disease is a leading cause of morbidity and mortality, and can affect nearly all tissues and organ systems. The United States government estimates that 45% of deaths in the United States can be attributed to fibrotic diseases.

### SUMMARY

This document provides flavonoid compounds and methods and materials for using flavonoid compounds to treat mammals (e.g., humans) having one or more fibrotic conditions (e.g., idiopathic pulmonary fibrosis (IPF), non-alcoholic steatohepatitis (NASH), primary sclerosing cholangitis (PSC), and/or ocular fibrosis). For example, this document provides flavonoid compounds having the structure of Formula (I) as well as methods and materials for using one or more flavonoid compounds having the structure of Formula (I). In some cases, one or more flavonoid compounds having the structure of Formula (I) can be administered to a mammal (e.g., a human) having one or more fibrotic conditions to treat that condition within the mammal. As demonstrated herein, one or more flavonoid compounds having the structure of Formula (I) can induce apoptosis in senescent cells (e.g., senescent fibroblasts), and can be used to treat a fibrotic condition within a mammal (e.g., a human).

In general, one aspect of this document features compositions including a flavonoid compound having a structure of Formula (I):

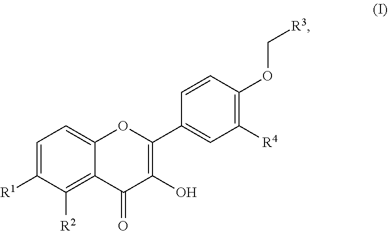

or a pharmaceutically acceptable salt thereof, where: R¹ is H, OH, a C₁-C₄ alkyl, a halogen, or a C₁-C₄ alkoxy; R² is H, OH, a C₁-C₄ alkyl, a halogen, or a C₁-C₄ alkoxy; R³ is H, CH₂CH₃, cyclopropyl, phenyl, 2-pyridinyl, 3-pyridinyl, or 4-pyridinyl; and R⁴ is H, OH, a C₁-C₄ alkyl, a halogen, or a C₁-C₄ alkoxy. The flavonoid compound of Formula (I) can have structure:

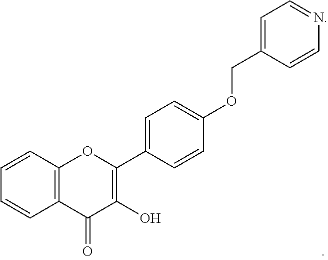 [SMILES: O=c1c(O)c(-c2ccc(OCc3ccncc3)cc2)oc2ccccc12]

The flavonoid compound of Formula (I) can have structure:

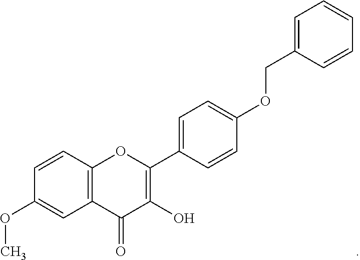 [SMILES: COc1ccc2oc(-c3ccc(OCc4ccccc4)cc3)c(O)c(=O)c2c1]

The flavonoid compound of Formula (I) can have structure:

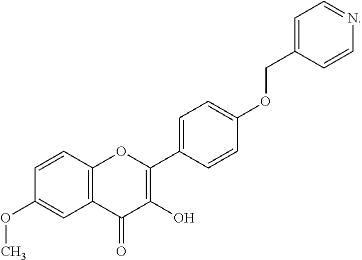 [SMILES: COc1ccc2oc(-c3ccc(OCc4ccncc4)cc3)c(O)c(=O)c2c1]

The composition also can include a pharmaceutically acceptable carrier, excipient, or diluent.

In another aspect, this document features pharmaceutical compositions including a flavonoid compound having a structure of Formula (I):

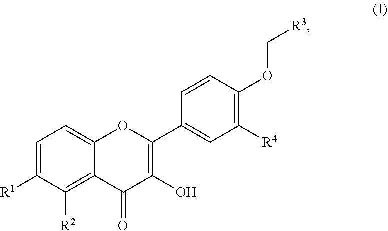

or a pharmaceutically acceptable salt thereof, where: R¹ is H, OH, a C₁-C₄ alkyl, a halogen, or a C₁-C₄ alkoxy; R² is H, OH, a C₁-C₄ alkyl, a halogen, or a C₁-C₄ alkoxy; R³ is H, CH₂CH₃, cyclopropyl, phenyl, 2-pyridinyl, 3-pyridinyl, or 4-pyridinyl; and R⁴ is H, OH, a C₁-C₄ alkyl, a halogen, or a C₁-C₄ alkoxy, and a pharmaceutically acceptable carrier, excipient, or diluent.

In another aspect, this document features methods for treating a mammal having a fibrotic condition. The methods can include, or consist essentially of, administering a composition including a flavonoid compound having a structure of Formula (I):

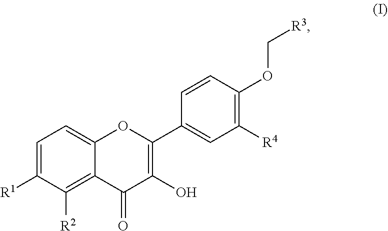

or a pharmaceutically acceptable salt thereof, where: R¹ is H, OH, a C₁-C₄ alkyl, a halogen, or a C₁-C₄ alkoxy; R² is H, OH, a C₁-C₄ alkyl, a halogen, or a C₁-C₄ alkoxy; R³ is H, CH₂CH₃, cyclopropyl, phenyl, 2-pyridinyl, 3-pyridinyl, or 4-pyridinyl; and R⁴ is H, OH, a C₁-C₄ alkyl, a halogen, or a C₁-C₄ alkoxy to a mammal having a fibrotic condition. The mammal can be a human. The method can include identifying the mammal as having the fibrotic condition. The fibrotic condition can be IPF, PSC, NASH, or ocular fibrosis. The fibrotic condition can be IPF, and the method can include administering an agent used to treat IPF to the mammal. The agent used to treat IPF can be pirfenidone, nintedanib, N-acetylcysteine, sildenafil, vardenafil, tadalafil, avanafil, promethazine, FTY720, AM152, BMS-986020, VPC 12249, AM966, AM095, taribavirin, BI-2545, GLPG1690, BBT 877, SAR100842, BMS-986,020, minaprine, dopamine, levodopa, apomorphine, fenoldopam, pergolide, bromocriptine, cabergoline, dasatinib, hydroxyfasudil, ripasudil, netarsudil, belumosudil, lebrikizumab, tralokinumab, dupilumab, or pamrevlumab. The fibrotic condition can be PSC, and the method can include administering an agent used to treat PSC to the mammal. The agent used to treat said PSC can be ursodeoxycholic acid (UDCA), a corticosteroid, a bile acid sequestrant, an antibiotic, or an antihistamine.

In another aspect, this document features methods for reducing fibrosis in a mammal having a fibrotic condition. The methods can include, or consist essentially of, administering a composition including a flavonoid compound having a structure of Formula (I):

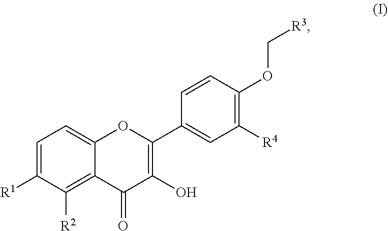

or a pharmaceutically acceptable salt thereof, where: R¹ is H, OH, a C₁-C₄ alkyl, a halogen, or a C₁-C₄ alkoxy; R² is H, OH, a C₁-C₄ alkyl, a halogen, or a C₁-C₄ alkoxy; R³ is H, CH₂CH₃, cyclopropyl, phenyl, 2-pyridinyl, 3-pyridinyl, or 4-pyridinyl; and R⁴ is H, OH, a C₁-C₄ alkyl, a halogen, or a C₁-C₄ alkoxy to a mammal having a fibrotic condition. The mammal can be a human. The method can include identifying the mammal as having the fibrotic condition. The fibrotic condition can be IPF, PSC, NASH, or ocular fibrosis.

In another aspect, this document features methods for reducing a number of senescent cells in a mammal having a fibrotic condition. The methods can include, or consist essentially of, administering a composition including a flavonoid compound having a structure of Formula (I):

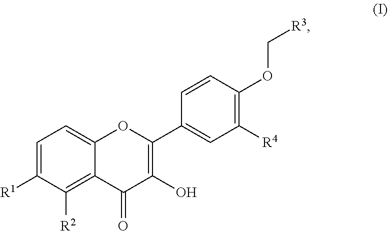

or a pharmaceutically acceptable salt thereof, where: R¹ is H, OH, a C₁-C₄ alkyl, a halogen, or a C₁-C₄ alkoxy; R² is H, OH, a C₁-C₄ alkyl, a halogen, or a C₁-C₄ alkoxy; R³ is H, CH₂CH₃, cyclopropyl, phenyl, 2-pyridinyl, 3-pyridinyl, or 4-pyridinyl; and R⁴ is H, OH, a C₁-C₄ alkyl, a halogen, or a C₁-C₄ alkoxy to a mammal having a fibrotic condition. The mammal can be a human. The method can include identifying the mammal as having the fibrotic condition. The fibrotic condition can be IPF, PSC, NASH, or ocular fibrosis. The senescent cell can be a fibroblast. The fibrotic condition can be IPF, and the senescent cell can be a lung fibroblast. The senescent cell can be an epithelial cell. The fibrotic condition can be PSC, and the senescent cell can be a cholangiocyte.

In another aspect, this document features methods for inhibiting a serine/threonine kinase 17 (STK17) polypeptide in a mammal. The methods can include, or consist essentially of, administering a composition including a flavonoid compound having a structure of Formula (I):

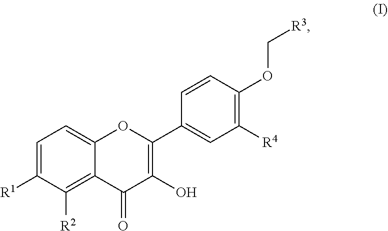

or a pharmaceutically acceptable salt thereof, where: R¹ is H, OH, a C₁-C₄ alkyl, a halogen, or a C₁-C₄ alkoxy; R² is H, OH, a C₁-C₄ alkyl, a halogen, or a C₁-C₄ alkoxy; R³ is H, CH₂CH₃, cyclopropyl, phenyl, 2-pyridinyl, 3-pyridinyl, or 4-pyridinyl; and R⁴ is H, OH, a C₁-C₄ alkyl, a halogen, or a C₁-C₄ alkoxy to a mammal. The mammal can be a human. The STK17 polypeptide can be a STK17A (DRAK1) polypeptide or a STK17B (DRAK2) polypeptide.

In another aspect, this document features compositions including a flavonoid compound having a structure of Formula (II):

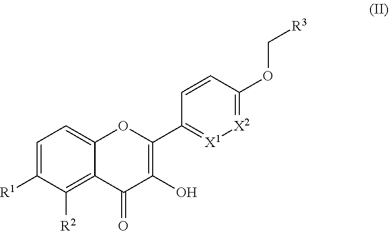

or a pharmaceutically acceptable salt thereof, wherein X¹ is selected from N and CH; X² is selected from N and CR⁴; R¹ is selected from the group consisting of H, OH, a C₁-C₄ alkyl, a halogen, and a C₁-C₄ alkoxy; R² is selected from the group consisting of H, OH, a C₁-C₄ alkyl, a halogen, and a C₁-C₄ alkoxy; R³ is selected from the group consisting of H, CH₃, CH₂CH₃, cyclopropyl, phenyl, 4-OH-phenyl, 2-OH-phenyl, 3-OH-phenyl, 2-pyridinyl, 3-pyridinyl, 4-pyridinyl, thiophen-2-yl, thiophen-3-yl, tetrahydrofuran-2-yl, and tetrahydrofuran-3-yl; and R⁴ is selected from the group consisting of H, OH, a C₁-C₄ alkyl, a halogen, and a C₁-C₄ alkoxy. The flavonoid compound of Formula (II) has any one of the following formulae:

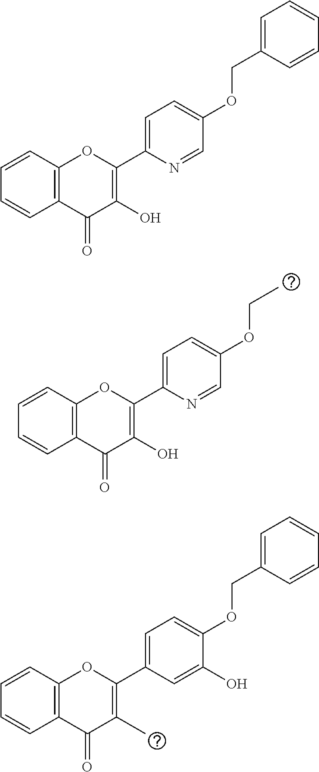 [SMILES: O=c1c(O)c(-c2ccc(OCc3ccccc3)cn2)oc2ccccc12 ; CCOc1ccc(-c2oc3ccccc3c(=O)c2O)nc1 ; Cc1c(-c2ccc(OCc3ccccc3)c(O)c2)oc2ccccc2c1=O]

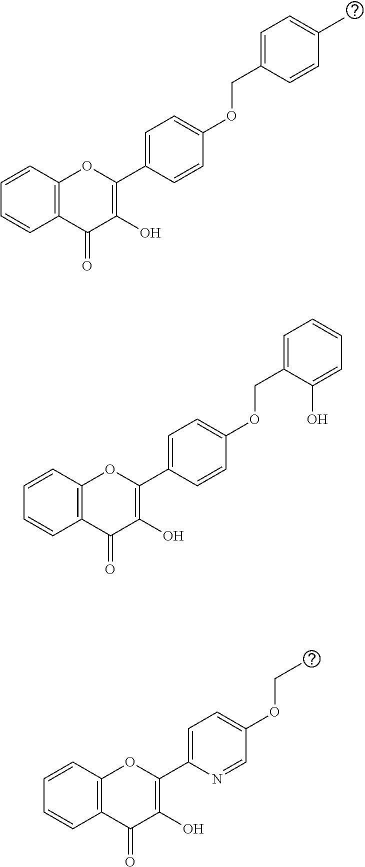 [SMILES: Cc1ccc(COc2ccc(-c3oc4ccccc4c(=O)c3O)cc2)cc1 ; O=c1c(O)c(-c2ccc(OCc3ccccc3O)cc2)oc2ccccc12 ; CCOc1ccc(-c2oc3ccccc3c(=O)c2O)nc1]

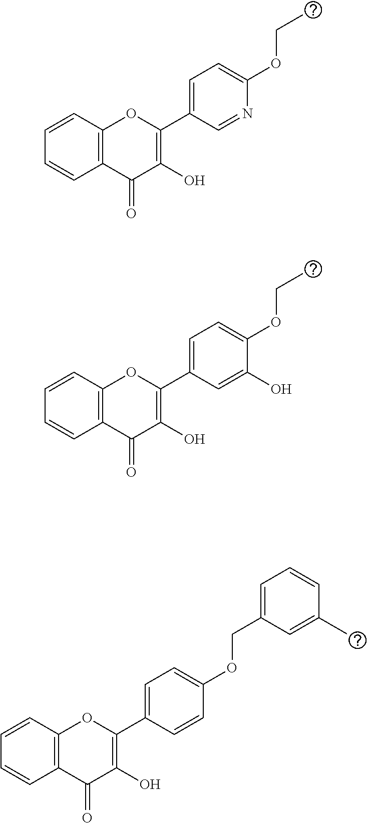 [SMILES: CCOc1ccc(-c2oc3ccccc3c(=O)c2O)cn1 ; CCOc1ccc(-c2oc3ccccc3c(=O)c2O)cc1O ; Cc1cccc(COc2ccc(-c3oc4ccccc4c(=O)c3O)cc2)c1]

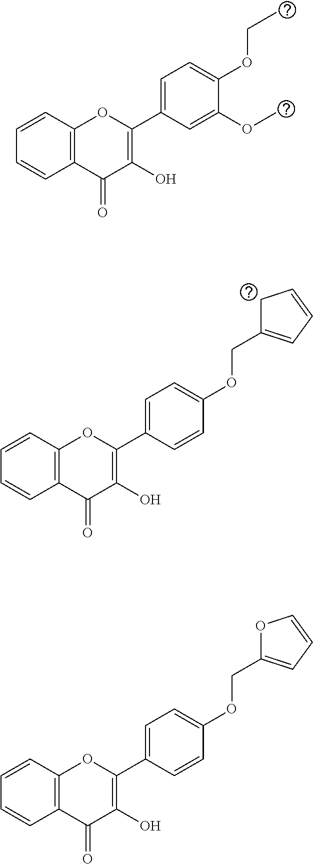 [SMILES: CCOc1ccc(-c2oc3ccccc3c(=O)c2O)cc1OC ; O=c1c(O)c(-c2ccc(OCC3=CC=CC3)cc2)oc2ccccc12 ; O=c1c(O)c(-c2ccc(OCc3ccco3)cc2)oc2ccccc12]

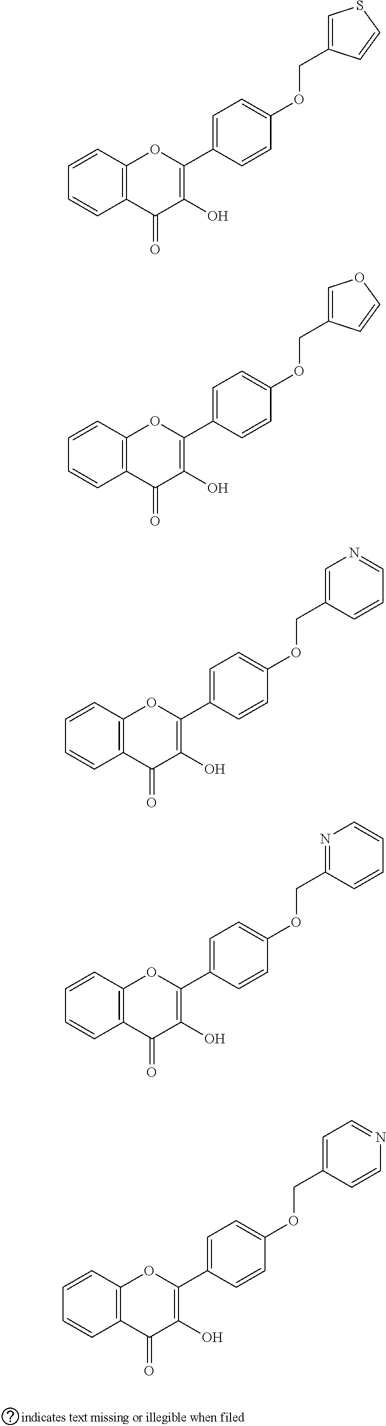 [SMILES: O=c1c(O)c(-c2ccc(OCc3ccsc3)cc2)oc2ccccc12 ; O=c1c(O)c(-c2ccc(OCc3ccoc3)cc2)oc2ccccc12 ; O=c1c(O)c(-c2ccc(OCc3cccnc3)cc2)oc2ccccc12 ; O=c1c(O)c(-c2ccc(OCc3ccccn3)cc2)oc2ccccc12 ; O=c1c(O)c(-c2ccc(OCc3ccncc3)cc2)oc2ccccc12]

or a pharmaceutically acceptable salt thereof. The composition can be a pharmaceutical composition. The pharmaceutical composition can include a pharmaceutically acceptable carrier, excipient, or diluent.

In another aspect, this document features methods for treating a mammal having a fibrotic condition. The methods can include, or consist essentially of, administering a composition including a flavonoid compound having a structure of Formula (II):

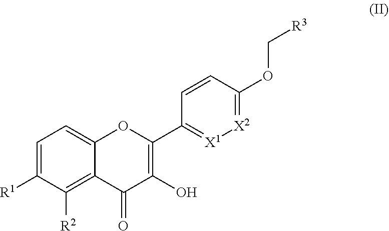

or a pharmaceutically acceptable salt thereof, wherein X¹ is selected from N and CH; X² is selected from N and CR⁴; R¹ is selected from the group consisting of H, OH, a C₁-C₄ alkyl, a halogen, and a C₁-C₄ alkoxy; R² is selected from the group consisting of H, OH, a C₁-C₄ alkyl, a halogen, and a C₁-C₄ alkoxy; R³ is selected from the group consisting of H, CH₃, CH₂CH₃, cyclopropyl, phenyl, 4-OH-phenyl, 2-OH-phenyl, 3-OH-phenyl, 2-pyridinyl, 3-pyridinyl, 4-pyridinyl, thiophen-2-yl, thiophen-3-yl, tetrahydrofuran-2-yl, and tetrahydrofuran-3-yl; and R⁴ is selected from the group consisting of H, OH, a C₁-C₄ alkyl, a halogen, and a C₁-C₄ alkoxy to a mammal having a fibrotic condition. The mammal can be a human. The method can include identifying the mammal as having said fibrotic condition. The fibrotic condition can be IPF, PSC, NASH, or ocular fibrosis. The fibrotic condition can be IPF, and the method also can include administering an agent used to treat IPF to said mammal. The agent used to treat said IPF can be pirfenidone, nintedanib, N-acetylcysteine, sildenafil, vardenafil, tadalafil, avanafil, promethazine, FTY720, AM152, BMS-986020, VPC 12249, AM966, AM095, taribavirin, BI-2545, GLPG1690, BBT 877, SAR100842, BMS-986,020, minaprine, dopamine, levodopa, apomorphine, fenoldopam, pergolide, bromocriptine, cabergoline, dasatinib, hydroxyfasudil, ripasudil, netarsudil, belumosudil, lebrikizumab, tralokinumab, dupilumab, or pamrevlumab. The fibrotic condition can be PSC, and the method also can include administering an agent used to treat PSC to said mammal. The agent used to treat said PSC can be UDCA, a corticosteroid, a bile acid sequestrant, an antibiotic, or an antihistamine.

In another aspect, this document features methods for reducing fibrosis in a mammal having a fibrotic condition. The methods can include, or consist essentially of, administering a composition including a flavonoid compound having a structure of Formula (II):

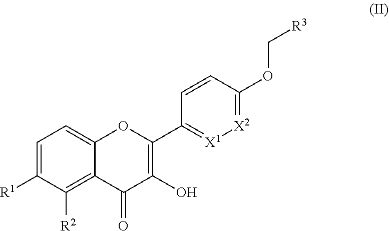

or a pharmaceutically acceptable salt thereof, wherein X¹ is selected from N and CH; X² is selected from N and CR⁴; R¹ is selected from the group consisting of H, OH, a C₁-C₄ alkyl, a halogen, and a C₁-C₄ alkoxy; R² is selected from the group consisting of H, OH, a C₁-C₄ alkyl, a halogen, and a C₁-C₄ alkoxy; R³ is selected from the group consisting of H, CH₃, CH₂CH₃, cyclopropyl, phenyl, 4-OH-phenyl, 2-OH-phenyl, 3-OH-phenyl, 2-pyridinyl, 3-pyridinyl, 4-pyridinyl, thiophen-2-yl, thiophen-3-yl, tetrahydrofuran-2-yl, and tetrahydrofuran-3-yl; and R⁴ is selected from the group consisting of H, OH, a C₁-C₄ alkyl, a halogen, and a C₁-C₄ alkoxy to a mammal having a fibrotic condition. The mammal can be a human. The method can include identifying the mammal as having said fibrotic condition. The fibrotic condition can be IPF, PSC, NASH, or ocular fibrosis.

In another aspect, this document features methods for reducing a number of senescent cells in a mammal having a fibrotic condition. The methods can include, or consist essentially of, administering a composition including a flavonoid compound having a structure of Formula (II):

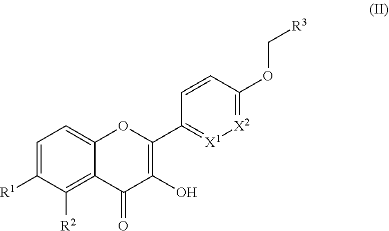

or a pharmaceutically acceptable salt thereof, wherein X¹ is selected from N and CH; X² is selected from N and CR⁴; R¹ is selected from the group consisting of H, OH, a C₁-C₄ alkyl, a halogen, and a C₁-C₄ alkoxy; R² is selected from the group consisting of H, OH, a C₁-C₄ alkyl, a halogen, and a C₁-C₄ alkoxy; R³ is selected from the group consisting of H, CH₃, CH₂CH₃, cyclopropyl, phenyl, 4-OH-phenyl, 2-OH-phenyl, 3-OH-phenyl, 2-pyridinyl, 3-pyridinyl, 4-pyridinyl, thiophen-2-yl, thiophen-3-yl, tetrahydrofuran-2-yl, and tetrahydrofuran-3-yl; and R⁴ is selected from the group consisting of H, OH, a C₁-C₄ alkyl, a halogen, and a C₁-C₄ alkoxy to a mammal having a fibrotic condition. The mammal can be a human. The method can include identifying the mammal as having said fibrotic condition. The fibrotic condition can be IPF, PSC, NASH, and ocular fibrosis. The senescent cell can be a fibroblast. The fibrotic condition can be IPF, and the senescent cell can be a lung fibroblast. The senescent cell can be an epithelial cell. The fibrotic condition can be PSC, and the senescent cell can be a cholangiocyte.

In another aspect, this document features methods for inhibiting a STK17 polypeptide in a mammal. The methods can include, or consist essentially of, administering a composition including a flavonoid compound having a structure of Formula (II):

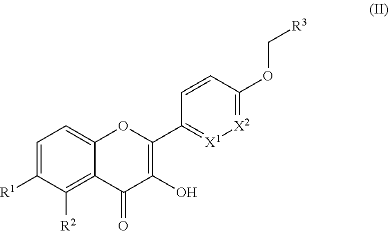

or a pharmaceutically acceptable salt thereof, wherein X¹ is selected from N and CH; X² is selected from N and CR⁴; R¹ is selected from the group consisting of H, OH, a C₁-C₄ alkyl, a halogen, and a C₁-C₄ alkoxy; R² is selected from the group consisting of H, OH, a C₁-C₄ alkyl, a halogen, and a C₁-C₄ alkoxy; R³ is selected from the group consisting of H, CH₃, CH₂CH₃, cyclopropyl, phenyl, 4-OH-phenyl, 2-OH-phenyl, 3-OH-phenyl, 2-pyridinyl, 3-pyridinyl, 4-pyridinyl, thiophen-2-yl, thiophen-3-yl, tetrahydrofuran-2-yl, and tetrahydrofuran-3-yl; and R⁴ is selected from the group consisting of H, OH, a C₁-C₄ alkyl, a halogen, and a C₁-C₄ alkoxy to a mammal. The mammal can be a human. The STK17 polypeptide can be a STK17A (DRAK1) polypeptide or a STK17B (DRAK2) polypeptide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 shows a schematic representation of an exemplary chemical synthesis of flavonoid compounds having the structure of Formula (I).

FIG. 2 shows effects of F-19 on resident lung cell populations following bleomycin injury and lung fibrosis. Seven days prior to injury col1a2-mTmG mice were treated with tamoxifen to turn on GFP expression in collagen producing fibroblasts. F-19 was administered daily starting on day 10 after injury, lungs were isolated, and lung tissue was flow sorted for fibroblasts, epithelial cells, and leukocytes on day 14. The sorted cells were subjected to RNA isolation followed by qPCR analysis of the indicated genes in each population of cells. N=3 mice/group. No changes in senescence markers or inflammatory cytokines were measured in leukocytes (CD45⁺ cells), suggesting selectivity.

FIGS. 3A-3C show efficacy of F-4N in a non-resolving model of lung fibrosis (aged mice). FIG. 3A is a graph showing survival of the mouse groups. 10-18-month-old C57/B6 mice received intratracheal bleomycin on day 0, and on day 14 one group started receiving F-4N (10 mg/kg daily i.p.). FIG. 3B contains images showing lung histology (trichrome staining) of mouse groups (Sham, Bleo+vehicle, and Bleo+F-4N), and a bar graph showing hydroxyproline content analysis. FIG. 3C contains bar graphs showing whole lung expression of collagen I and senescence markers (Cdkn2a, Cdkn1a, and Ccl2). N=7-9 male and female mice.

FIGS. 4A-4B show mechanism of action of F-4N. FIG. 4A shows a Z-score plot of kinome screen of 1 μM F-4N against ˜400 kinases. Dose response curves on STK17A and STK17B show half maximal inhibitory concentration (IC₅₀) values of ˜200 nM. FIG. 4B contains a graph showing expression of putative targets in senescent lung fibroblasts and show that STK17A/B are dramatically overexpressed.

FIGS. 5A-5B contain bar graphs comparing the effect of siRNA targeting STK17A/B in senescent lung fibroblasts (etoposide induced) and proliferative lung fibroblasts (pro), and show that STK17A/B selectively regulate senescent lung fibroblasts. FIG. 5A contains bar graphs showing the relative cell numbers per field of view for senescent (sen) or proliferative (pro) cells transfected with non-targeting (NT) siRNA or STK17A and STK17B (STK17A/B) siRNA. N=2. After 4 days, cells were fixed and stained for DAPI (cell count) and cleaved caspase-3 (apoptosis). FIG. 5B contains bar graphs showing cells counted by automated imaging software. Cells were transfected; after 4 days, total RNA was isolated, and qPCR analysis performed. N=3.

FIG. 6. Biochemical and histological examination of fibrosis in Mdr2^{-/-} mice treated with vehicle or F-4N. Mdr2^{-/-} mice (18-20 weeks old) with established liver fibrosis were treated with F-4N (10 mg/kg/daily i.p. n=10) or vehicle (i.p. n=10) for 14 days, then sacrificed the next day. Liver tissue and serum were collected for analysis. Left panel. Hydroxyproline assays on liver tissue revealed that F-4N treatment reduced collagen content by ˜40% compared to vehicle control-treated mice. Right panels. Picrosirius red staining of mouse liver that selectively visualizes collagen fibers shows reduction of collagen fibers in F-4N treated mice, particularly in the portal-to-portal areas (“bridging fibrosis”).

FIG. 7. Liver function tests are improved in Mdr2^{-/-} mice following F-4N treatment. Alanine aminotransferase (ALT), a marker of liver injury, and alkaline phosphatase (ALP) and serum bile acid values (markers of biliary injury and cholestasis) were significantly reduced in Mdr2^{-/-} mice treated with F-4N compared to vehicle-treated Mdr2^{-/-} mice.

FIG. 8. Reverse transcriptase (RT)-PCR of whole liver RNA shows decreased markers of fibrosis, inflammation and senescence. RT-PCR was performed for the fibrosis marker collagen 1A1 (Col1a1), the inflammatory markers Interleukin 6 (Il6) and C-C Motif Chemokine Ligand 2 (Ccl2), and the senescence markers Cyclin-Dependent Kinase Inhibitor 1A (Cdkn1a) and 2A (Cdkn2a). Each marker was significantly reduced in Mdr2^{-/-} mice treated with F-4N compared to vehicle-treated Mdr2^{-/-} mice.

FIGS. 9A-9R contain 3 point dose response curves of senescent fibroblasts and healthy fibroblasts treated with flavonols. Senescent fibroblasts (circle data points) or TGFB stimulated collagen deposition in low passage lung fibroblasts (square datapoints) were treated with either escalating doses of either (FIG. 9A) quercetin, (FIG. 9B) fisetin, (FIG. 9C) 2-(3,4-dimethoxyphenyl)-3-hydroxy-6,8-dimethylchromen-4-one, (FIG. 9D) 6-chloro-2-(3,4-dimethoxyphenyl)-3-hydroxy-4h-1-benzopyran-4-one, (FIG. 9E) flavonol, (FIG. 9F) 4′-methoxy-3-flavonol, (FIG. 9G) 4′-hydroxy-3′-methoxy-flavone, (FIG. 9H) 3-hydroxy-2-(4-methoxyphenyl)-6-methyl-4h-1-benzopyran-4-one, (FIG. 91) 2-(3,4-dimethoxyphenyl)-3-hydroxy-6-methyl-4h-1-benzopyran-4-one, (FIG. 9J) 2-(4-ethoxy-3-methoxyphenyl)-3-hydroxy-6-methylchromen-4-one, (FIG. 9K) 3-hydroxy-6-methyl-flavone, (FIG. 9L) 3-hydroxy-2-(3-methoxyphenyl)-4h-1-benzopyran-4-one, (FIG. 9M) 3′,4′-dihydroxy-flavone, (FIG. 9N) 3-hydroxy-7-(phenylmethoxy)-2-(3,4,5-trimethoxyphenyl)-4h-1-benzopyran-4-one, (FIG. 9O) 2-(3,4-dimethoxyphenyl)-3-hydroxy-7-methyl-4h-1-benzopyran-4-one, (FIG. 9P) 3-hydroxy-3′,4′-(methylenedioxy)-flavone, (FIG. 9Q) 2-(3,4-diethoxyphenyl)-3-hydroxy-4h-1-benzopyran-4-one, or (FIG. 9R) 3-hydroxy-3′,4′-dimethoxyflavone. For senescent fibroblast survival experiments (circle datapoints), human adult lung fibroblasts were replicatively passaged until senescence (confirmed by RNA and senescence associated beta galactosidase staining). Cells were then plated into 96-well plates and treated with the indicated compound and incubated for 96 hours. Cells were then fixed using 4% PFA, permeabilized using 0.25% triton x-100, and nuclear stained using DAPI. Cells were then imaged using a 4× objective on a Cytation5 microscope and cell counts were quantified using automated software (Biotek Gen5). Data shown are plotted as % survival, normalized to the vehicle treated well. Mean+/−SEM, N=3 independent experiments. For the TGFβ stimulated collagen deposition experiments (square datapoints), human adult lung fibroblasts (passage 3) were plated into 96-well plates and treated with the indicated compound +2 ng/mL TGFβ to stimulated collagen expression. Cells were incubated for 96 hours, fixed using 4% PFA, permeabilized using 0.25% triton x-100, and stained using a primary antibody recognizing type I collagen and a secondary infrared conjugated antibody. Wells were then imaged at 1× using a Odyssey Lx (LI-CORE) infrared imager and collagen intensity was quantified using automated software. Data shown are plotted as % collagen intensity, normalized to the vehicle treated well. Mean+/−SEM, N=3 independent experiments.

FIGS. 10A-10B contain 6 point dose response curves of senescent fibroblasts and healthy fibroblasts treated with flavonols. Senescent fibroblasts (circle data points) or TGFB stimulated collagen deposition in low passage lung fibroblasts (square datapoints) were treated with either escalating doses of either (FIG. 10A) 2-(4-ethoxy-3-methoxyphenyl)-3-hydroxy-6-methylchromen-4-one or (FIG. 10B) 2-(3,4-diethoxyphenyl)-3-hydroxy-4h-1-benzopyran-4-one. For senescent fibroblast survival experiments (circle datapoints), human adult lung fibroblasts were replicatively passaged until senescence (confirmed by RNA and senescence associated beta galactosidase staining). Cells were then plated into 96-well plates and treated with the indicated compound and incubated for 96 hours. Cells were then fixed using 4% PFA, permeabilized using 0.25% triton x-100, and nuclear stained using DAPI. Cells were then imaged using a 4× objective on a Cytation5 microscope and cell counts were quantified using automated software (Biotek Gen5). Data shown are plotted as % survival, normalized to the vehicle treated well. Mean+/−SEM, N=3 independent experiments. For the TGFβ stimulated collagen deposition experiments (square datapoints), human adult lung fibroblasts (passage 3) were plated into 96-well plates and treated with the indicated compound +2 ng/mL TGFβ to stimulated collagen expression. Cells were incubated for 96 hours, fixed using 4% PFA, permeabilized using 0.25% triton x-100, and stained using a primary antibody recognizing type I collagen and a secondary infrared conjugated antibody. Wells were then imaged at 1× using a Odyssey Lx (LI-CORE) infrared imager and collagen intensity was quantified using automated software. Data shown are plotted as % collagen intensity, normalized to the vehicle treated well. Mean+/−SEM, N=3 independent experiments.

FIGS. 11A-11J contain 6 point dose response curves of senescent fibroblasts and healthy fibroblasts treated with flavonols modified with para ethoxy derivatives. Senescent fibroblasts (circle data points) or TGFB stimulated collagen deposition in low passage lung fibroblasts (square datapoints) were treated with either escalating doses of either (FIG. 11A) compound 17, (FIG. 11B) compound 18, (FIG. 11C) compound 19, (FIG. 11D) compound 20, (FIG. 11E) compound 21, (FIG. 11F) compound 22, (FIG. 11G) compound 23, (FIG. 11H) compound 24, (FIG. 11I) compound 25, or (FIG. 11J) compound 26. For senescent fibroblast survival experiments (circle datapoints), human adult lung fibroblasts were replicatively passaged until senescence (confirmed by RNA and senescence associated beta galactosidase staining (not shown)). Cells were then plated into 96-well plates and treated with the indicated compound and incubated for 96 hours. Cells were then fixed using 4% PFA, permeabilized using 0.25% triton x-100, and nuclear stained using DAPI. Cells were then imaged using a 4× objective on a Cytation5 microscope and cell counts were quantified using automated software (Biotek Gen5). Data shown are plotted as % survival, normalized to the vehicle treated well. Mean+/−SEM, N=3 independent experiments. For the TGFβ stimulated collagen deposition experiments (square datapoints), human adult lung fibroblasts (passage 3) were plated into 96-well plates and treated with the indicated compound +2 ng/mL TGFβ to stimulated collagen expression. Cells were incubated for 96 hours, fixed using 4% PFA, permeabilized using 0.25% triton x-100, and stained using a primary antibody recognizing type I collagen and a secondary infrared conjugated antibody. Wells were then imaged at 1× using a Odyssey Lx (LI-CORE) infrared imager and collagen intensity was quantified using automated software. Data shown are plotted as % collagen intensity, normalized to the vehicle treated well. Mean+/−SEM, N=3 independent experiments.

FIGS. 12A-12E contain 6 point dose response curves of senescent fibroblasts and healthy fibroblasts treated with flavonols with derivatized flavonol cores. Senescent fibroblasts (circle data points) or TGFB stimulated collagen deposition in low passage lung fibroblasts (square datapoints) were treated with either escalating doses of either (FIG. 12A) compound 27, (FIG. 12B) compound 28, (FIG. 12C) compound 29, (FIG. 12D) compound 30, or (FIG. 12E) compound 31. For senescent fibroblast survival experiments (circle datapoints), human adult lung fibroblasts were replicatively passaged until senescence (confirmed by RNA and senescence associated beta galactosidase staining (not shown)). Cells were then plated into 96-well plates and treated with the indicated compound and incubated for 96 hours. Cells were then fixed using 4% PFA, permeabilized using 0.25% triton x-100, and nuclear stained using DAPI. Cells were then imaged using a 4× objective on a Cytation5 microscope and cell counts were quantified using automated software (Biotek Gen5). Data shown are plotted as % survival, normalized to the vehicle treated well. Mean+/−SEM, N=3 independent experiments. For the TGFβ stimulated collagen deposition experiments (square datapoints), human adult lung fibroblasts (passage 3) were plated into 96-well plates and treated with the indicated compound +2 ng/mL TGFβ to stimulated collagen expression. Cells were incubated for 96 hours, fixed using 4% PFA, permeabilized using 0.25% triton x-100, and stained using a primary antibody recognizing type I collagen and a secondary infrared conjugated antibody. Wells were then imaged at 1× using a Odyssey Lx (LI-CORE) infrared imager and collagen intensity was quantified using automated software. Data shown are plotted as % collagen intensity, normalized to the vehicle treated well. Mean+/−SEM, N=3 independent experiments.

FIGS. 13A-13B show the efficacy of F-4N in a bleomycin injury model of lung fibrosis. FIG. 13A. Study design. Mice received intratracheal bleomycin treatment 3× every 2 weeks. 28 days after the last bleomycin injury mice were treated with vehicle of F-4N daily for 14 days. FIG. 13B. Representative H&E stained histological images of lungs from each group and hydroxyproline analysis of each lung from each group. N=3 sham mice, N=6 bleo vehicle. N=7 bleo F-4N. 8-week-old FVB wildtype mice.

FIG. 14. Whole lung RNA expression from repeated bleomycin injury study. Mouse lungs from FIG. 1 were analyzed by qPCR for changes in profibrotic genes, senescence associated genes, alveolar epithelial type I/type II markers, intermediate/transitional alveolar markers unique to fibrosis, and Stk17b. N=3 sham mice, N=6 bleo vehicle. N=7 bleo F-4N. 8-week-old FVB wildtype mice.

FIG. 15 contains a schematic of the study design used for Example 8.

FIG. 16 contains graphs showing that liver mass was reduced in Mdr2^{-/-} mice after oral treatment with F-4N.

FIGS. 17A-17B show that liver fibrosis was reduced in Mdr2^{-/-} mice after oral treatment with F-4N. FIG. 17A) Microscope images of liver tissues. FIG. 17B) Graphs showing amount of hydroxyproline (HYP) in livers.

FIG. 18 contains graphs showing that oral delivery of F-4N reduced markers of liver fibrosis in Mdr2^{-/-} mice.

FIG. 19 contains graphs showing that oral delivery of F-4N significantly reduces markers of liver inflammation and cellular senescence in Mdr2^{-/-} mice.

FIGS. 20A-20D show the efficacy of F-4N in a mouse model of NASH. FIG. 20A) Study design and weight changes of mice treated with 10 mg/kg F-4N daily i.p. for 2 weeks. FIG. 20B). Representative H&E and Sirius Red liver histology. FIG. 20C) Objective automated quantification of Sirius Red staining. FIG. 20D) Liver weight and colon weight changes. N=5 chow diet, N=10 holine deficient-high fat diet (CD-HFD) vehicle, N=10 CD-HFD+F-4N.

FIG. 21 shows an analysis of whole liver RNA. Livers from the study of FIG. 20 were analyzed by qPCR for expression of profibrotic and inflammatory genes, and expression of Stk17b the molecular target of F-4N.

FIGS. 22A-22C shows further analysis of samples from the study of FIG. 20 including liver function tests (FIG. 22A), liver triglyceride analysis (FIG. 22B), and hydroxyproline assessment of liver collagen content (FIG. 22C).

FIGS. 23A-23C shows in vitro efficacy of F-4N in ocular fibrosis models. FIG. 23A) Conjunctival fibroblasts cultured with or without fetal bovine serum (FBS) and with or without F-4N. N=3. FIG. 23B) Conjunctival fibroblasts cultured for 3 days+/−2 ng/ml TGFβ and the indicated concentration of 4N, cells are fixed, stained with DAPI and an antibody for αSMA, then quantified using automated software on a Cytation 5. N=3. FIG. 23C) Conjunctival fibroblasts cultured for 6 days+/−2 ng/ml TGFβ and 2% FBS, and the indicated concentration of 4N, cells are fixed, stained with DAPI and an antibody for collagen I, then quantified using automated software on a Cytation 5. N=3.

FIGS. 24A-24C shows an acute exposure bleomycin study with varying doses of F-4N. FIG. 24A) A schematic of the study protocol. On day 1 mice received intratracheal sham or bleomycin injury. On day 7 mice were divided into groups: vehicle, 10, 30, and 100 mg/kg F-4N treated daily by oral gavage, for 7 days. On day 14 organs and plasma were collected. FIG. 24B) Weight changes during the experiment. FIG. 24C) Whole lung RNA expression of profibrotic genes.

FIG. 25 shows a discovery of F-4N efficacy biomarkers. Mice were treated with vehicle or 30 mg/kg F-4N, and RNA was analyzed. Shown are 11 genes having reduced expression in mice after 7 days of treatment with F-4N.

FIG. 26 shows levels of F-4N in the plasma (top) and liver (bottom) of mice following F-4N exposure.

FIG. 27 shows stability of F-4N in plasma. Compounds were incubated with human (top) or mouse (bottom) plasma for the indicated amount of time. Warfarin was used as a control for stable compound, and propantheline was used as a control for an unstable compound.

FIG. 28 shows microsomal stability of F-4N. Compounds were incubated with human (top) or mouse (bottom) liver derived microsomes for the indicated amount of time. Verapamil was used as a control for a rapidly degraded compound.

FIGS. 29A-29B show results of Stk17b knockdown in a precision cut lung slice (PCLS) model. FIG. 29A) A schematic of the study protocol. PCLSs were cultured ex vivo for 4 days with either non-targeting siRNA or Stk17b targeting siRNA. FIG. 29B) RNA was then collected and analyzed by qPCR.

FIG. 30 shows the results of a DRAK1 activity assay in the presence of F-4N (top) or an inactive analog 5-MeOH-F-4N (bottom).

FIGS. 31A-31B show efficacy of F-4N in an IPF patient-derived organotypic ex vivo culture. FIG. 31A) qPCR analysis of lung tissue slices. FIG. 31B) ELISA analysis of media IL-6. N=3 patient samples.

FIGS. 32A-32D contain graphs showing comparative flavonoid efficacy and lack of toxicity in aged brain and liver. Five-month-old and twenty-eight month old wild type mice were oral gavage treated with the indicated concentrations of flavonoids (F, Q, compound 19, or compound 20) or vehicle for four consecutive days and were euthanized one week later. Real-time PCR analysis suggests that low dose administration of compound 19 and/or 20 may reduce expression of a key senescence-activation gene, p16ink4a, in (FIG. 32A) brain and (FIG. 32B) liver more robustly than fisetin (F) or quercetin (Q), which have established senolytic effects at comparatively higher doses. Analysis of CD68 expression, an indicator of inflammatory activation, demonstrates a lack of drug-induced toxicity in brain (FIG. 32C) or liver (FIG. 32D).

FIGS. 33A-33C show that quercetin analogs can potently kill senescent fibroblasts. FIGS. 33A and 33B) Validation of replicative induced senescent fibroblasts as measure by expression of senescence markers (FIG. 33A) and by proliferation (FIG. 33B). FIG. 33C) Proliferation of low passage proliferative fibroblasts after treatment with quercetin analogs. Shown are the 5 representative derivatives exhibiting nanomolar to low micromolar potency.

FIGS. 34A-34B show that TGFβ and senescent cell conditioned media can promote fibroblast to myofibroblast transdifferentiation. FIG. 34A) Representative images observed by αSMA staining. FIG. 34B) Quercetin analogs potently prevent fibroblast activation. NCM: non-conditioned media (control). NCM+TGFβ: non-conditioned media+2 ng/mL TGFβ. CCM: conditioned media from normal lung fibroblasts. SASP-CM: conditioned media from senescent lung fibroblasts.

FIGS. 35A-35B show that quercetin analogs can prevent SASP-CM and TGFβ induced collagen deposition. FIG. 35A) TGFβ and senescent cell conditioned media promote collagen I deposition. FIG. 35B) Quercetin analogs potently prevent collagen I deposition. NCM: non-conditioned media (control). NCM+TGFβ: non-conditioned media+2 ng/mL TGFβ.

FIGS. 36A-36B show that quercetin analogs can prevent TGFβ induced profibrotic gene expression. FIG. 36A) TGFβ promotes profibrotic gene expression. FIG. 36B) Quercetin analogs potently block profibrotic gene expression. NCM: non-conditioned media (control). NCM+TGFβ: non-conditioned media+2 ng/mL TGFβ.

FIGS. 37A-37C show that quercetin analogs having a p-ethoxy have enhanced activity. FIG. 37A) Exemplary quercetin analogs having a p-ethoxy (highlighted). FIG. 37B) A graph of cell proliferation showing that a quercetin analog having a p-ethoxy induced cellular senescence. FIG. 37C) A graph of cell proliferation showing that a quercetin analog lacking a p-ethoxy did not induce cellular senescence.

### DETAILED DESCRIPTION

This document provides flavonoid compounds and methods and materials for using flavonoid compounds to treat mammals (e.g., humans) having one or more fibrotic conditions (e.g., IPF, NASH, PSC, and ocular fibrosis). For example, this document provides flavonoid compounds having the structure of Formula (I):

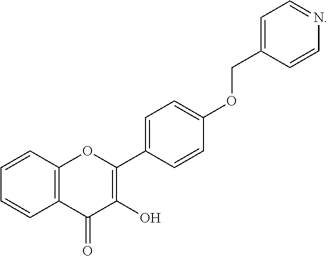 [SMILES: O=c1c(O)c(-c2ccc(OCc3ccncc3)cc2)oc2ccccc12]

where R¹ can be H, OH, a C₁-C₄ alkyl (e.g., methyl), a halogen, or a C₁-C₄ alkoxy (e.g., methoxy and ethoxy); R² can be H, OH, a C₁-C₄ alkyl (e.g., methyl), a halogen, or a C₁-C₄ alkoxy (e.g., methoxy and ethoxy); R³ can be H, CH₂CH₃, cyclopropyl, phenyl, 2-pyridinyl, 3-pyridinyl, or 4-pyridinyl; and R⁴ can be H, OH, a C₁-C₄ alkyl (e.g., methyl), a halogen, or a C₁-C₄ alkoxy (e.g., methoxy and ethoxy).

In some cases, a flavonoid compound provided herein can have the following structure and can be referred to as F-4N:

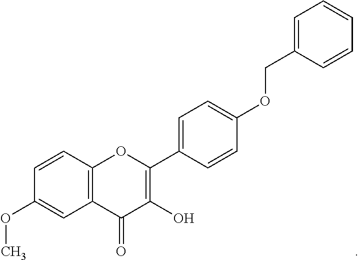 [SMILES: COc1ccc2oc(-c3ccc(OCc4ccccc4)cc3)c(O)c(=O)c2c1]

In some cases, a flavonoid compound provided herein can have the following structure and can be referred to as F-5MeO:

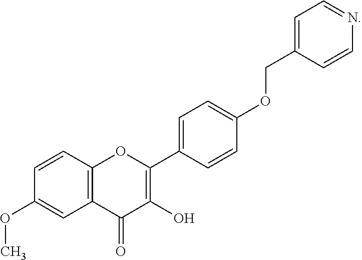 [SMILES: COc1ccc2oc(-c3ccc(OCc4ccncc4)cc3)c(O)c(=O)c2c1]

In some cases, a flavonoid compound provided herein can have the following structure and can be referred to as F-4N-5MeO:

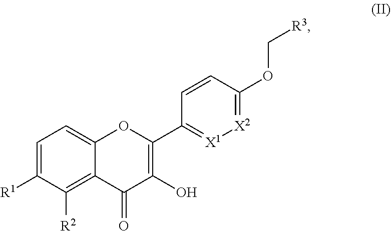

This document also provides flavonoid compounds and methods and materials for using flavonoid compounds to treat mammals (e.g., humans) having one or more fibrotic conditions (e.g., IPF, NASH, PSC, and ocular fibrosis). For example, this document provides flavonoid compounds having the structure of Formula (II):

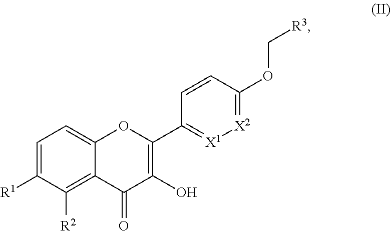

or a pharmaceutically acceptable salt thereof, wherein:
X¹ is selected from N and CH;
X² is selected from N and CR⁴;
R¹ is selected from the group consisting of H, OH, a C₁-C₄ alkyl, a halogen, and a C₁-C₄ alkoxy;
R² is selected from the group consisting of H, OH, a C₁-C₄ alkyl, a halogen, and a C₁-C₄ alkoxy;
R³ is selected from the group consisting of H, CH₃, CH₂CH₃, cyclopropyl, phenyl, 4-OH-phenyl, 2-OH-phenyl, 3-OH-phenyl, 2-pyridinyl, 3-pyridinyl, 4-pyridinyl, thiophen-2-yl, thiophen-3-yl, tetrahydrofuran-2-yl, and tetrahydrofuran-3-yl;
and R⁴ is selected from the group consisting of H, OH, a C₁-C₄ alkyl, a halogen, and a C₁-C₄ alkoxy.

In some embodiments, X¹ is N. In some embodiments, X¹ is CH. In some embodiments, X² is N. In some embodiments, X² is CR⁴. In some embodiments, R³ is H. In some embodiments, R³ is CH₃. In some embodiments, R³ is phenyl, 4-OH-phenyl, 2-OH-phenyl, or 3-OH-phenyl. In some embodiments, R³ is 2-pyridinyl, 3-pyridinyl, or 4-pyridinyl. In some embodiments, R³ is thiophen-2-yl, thiophen-3-yl, tetrahydrofuran-2-yl, or tetrahydrofuran-3-yl.

In some embodiments, the compound of Formula (II) has formula:

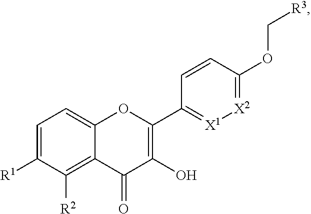

or a pharmaceutically acceptable salt thereof.

In some embodiments, the compound of Formula (II) has formula:

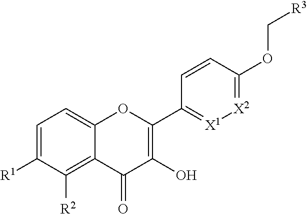

or a pharmaceutically acceptable salt thereof.

In some embodiments, the compound of Formula (II) is selected from any one of the following compounds:

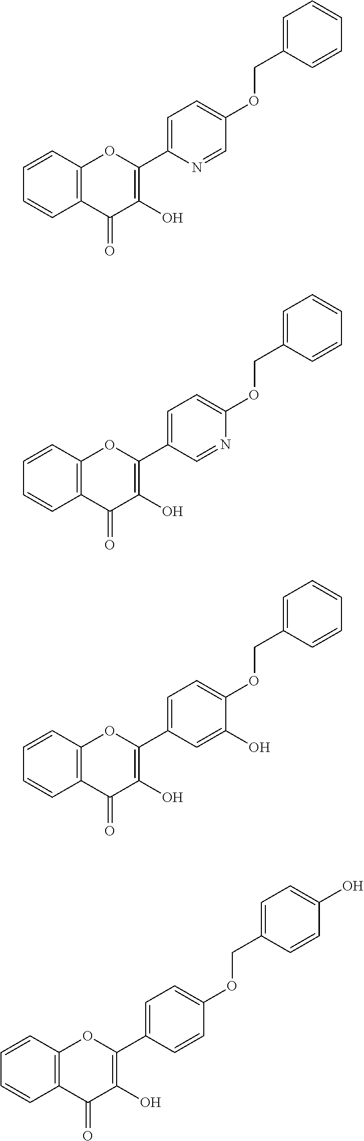 [SMILES: O=c1c(O)c(-c2ccc(OCc3ccccc3)cn2)oc2ccccc12 ; O=c1c(O)c(-c2ccc(OCc3ccccc3)nc2)oc2ccccc12 ; O=c1c(O)c(-c2ccc(OCc3ccccc3)c(O)c2)oc2ccccc12 ; O=c1c(O)c(-c2ccc(OCc3ccc(O)cc3)cc2)oc2ccccc12]

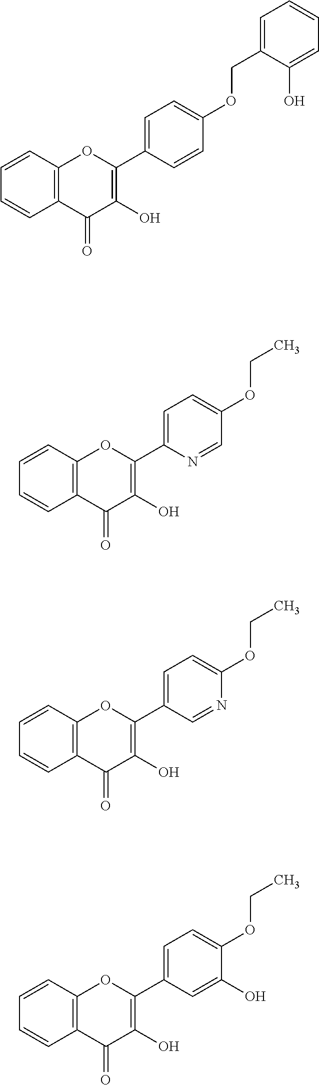 [SMILES: O=c1c(O)c(-c2ccc(OCc3ccccc3O)cc2)oc2ccccc12 ; CCOc1ccc(-c2oc3ccccc3c(=O)c2O)nc1 ; CCOc1ccc(-c2oc3ccccc3c(=O)c2O)cn1 ; CCOc1ccc(-c2oc3ccccc3c(=O)c2O)cc1O]

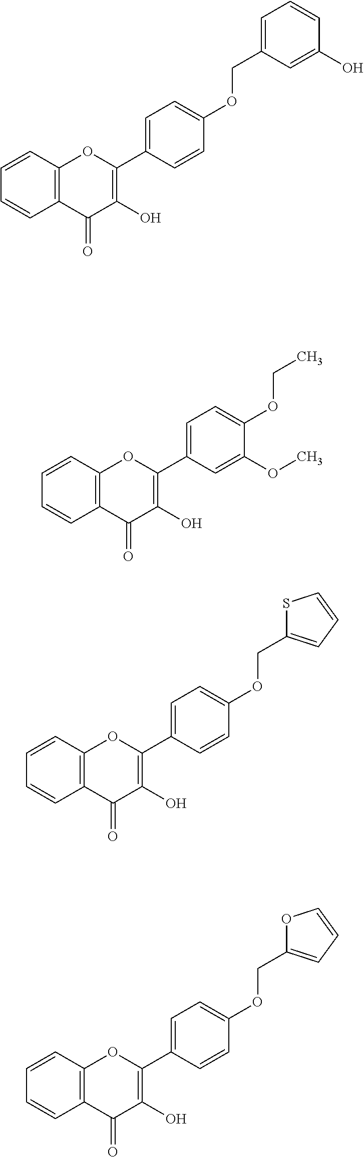 [SMILES: O=c1c(O)c(-c2ccc(OCc3cccc(O)c3)cc2)oc2ccccc12 ; CCOc1ccc(-c2oc3ccccc3c(=O)c2O)cc1OC ; O=c1c(O)c(-c2ccc(OCc3cccs3)cc2)oc2ccccc12 ; O=c1c(O)c(-c2ccc(OCc3ccco3)cc2)oc2ccccc12]

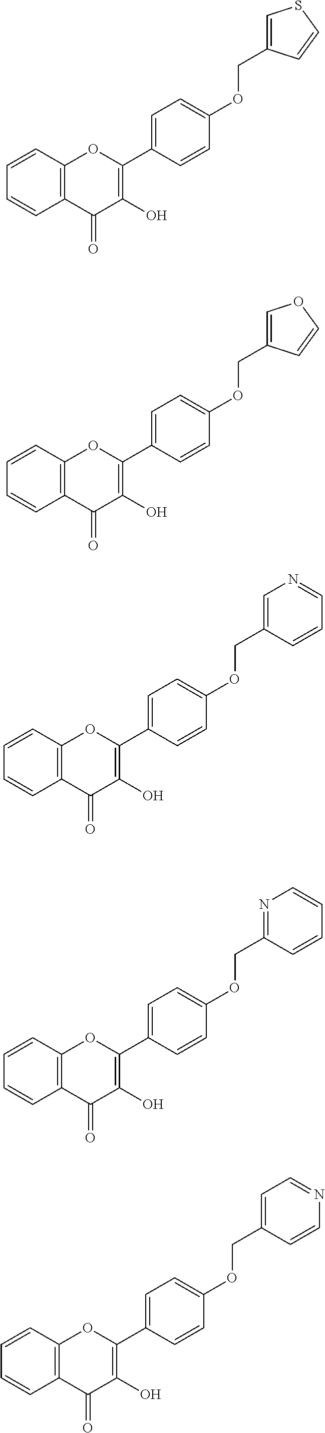 [SMILES: O=c1c(O)c(-c2ccc(OCc3ccsc3)cc2)oc2ccccc12 ; O=c1c(O)c(-c2ccc(OCc3ccoc3)cc2)oc2ccccc12 ; O=c1c(O)c(-c2ccc(OCc3cccnc3)cc2)oc2ccccc12 ; O=c1c(O)c(-c2ccc(OCc3ccccn3)cc2)oc2ccccc12 ; O=c1c(O)c(-c2ccc(OCc3ccncc3)cc2)oc2ccccc12]

or a pharmaceutically acceptable salt thereof.

In some cases, a flavonoid compound provided herein (e.g., a flavonoid compound having the structure of Formula (I) or Formula (II)) can be in the form of a salt (e.g., pharmaceutically acceptable salt). A salt of a compound provided herein can be formed between an acid and a basic group of the compound, such as an amino functional group, or a base and an acidic group of the compound, such as a carboxyl functional group. When a flavonoid compound having the structure of Formula (I) or Formula (II) is in the form of a salt, the salt can include any appropriate acid (e.g., an organic acid or an inorganic acid). Examples of acids that can be used to form a pharmaceutically acceptable salt of a compound described herein include, without limitation, inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts can be used including, without limitation, those formed with mineral acids such as hydrochloric acid and hydrobromic acid, and those formed with organic acids such as maleic acid. Examples of bases that can be used to form a pharmaceutically acceptable salt of a compound described herein include, without limitation, hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. In some cases, a compound described herein, or a pharmaceutically acceptable salt thereof, can be substantially isolated.

At various places herein, substituents of compounds described herein are described in groups or in ranges. It is specifically intended that this document include and describe each and every individual member or subcombination of the members of such groups and ranges. For example, the term “C₁-6 alkyl” is specifically intended to individually disclose methyl, ethyl, C₃ alkyl, C₄ alkyl, C₅ alkyl, and C₆ alkyl.

At various places herein, various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features herein, which are, for clarity, described in the context of separate embodiments, can also be provided in combination in a single embodiment. Conversely, various features herein which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cn-m” indicates a range which includes the endpoints, wherein n and m are integers and indicate the number of carbons. Examples include C₁₋₄, C₁₋₆, and the like.

As used herein, the term “Cn-m alkyl”, used alone or in combination with other terms, refers to a saturated hydrocarbon group that may be straight-chain or branched, having n to m carbons. Examples of alkyl moieties include, without limitation, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cn-m alkylene”, used alone or in combination with other terms, refers to a divalent alkyl linking group having n to m carbons. Examples of alkylene groups include, without limitation, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cn-m alkoxy”, used alone or in combination with other terms, refers to a group of formula-O-alkyl, wherein the alkyl group has n to m carbons. Example alkoxy groups include, without limitation, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include, without limitation, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include, without limitation, pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include, without limitation, ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some cases, one or more flavonoid compounds provided herein (e.g., one or more flavonoid compounds having the structure of Formula (I) or Formula (II)) can lack chirality.

In some cases, one or more flavonoid compounds provided herein (e.g., one or more flavonoid compounds having the structure of Formula (I) or Formula (II)) can be a neutral molecule (e.g., can lack any charged moiety).

In some cases, one or more flavonoid compounds provided herein (e.g., one or more flavonoid compounds having the structure of Formula (I) or Formula (II)) can lack any catechol moiety.

In some cases, one or more flavonoid compounds provided herein (e.g., one or more flavonoid compounds having the structure of Formula (I) or Formula (II)) can be formulated into a composition (e.g., a pharmaceutically acceptable composition) for administration to a mammal (e.g., a human) having one or more fibrotic conditions (e.g., IPF, NASH, PSC, and ocular fibrosis). For example, one or more flavonoid compounds having the structure of Formula (I) or Formula (II) can be formulated together with one or more pharmaceutically acceptable carriers (additives), excipients, and/or diluents. Examples of pharmaceutically acceptable carriers, excipients, and diluents that can be used in a composition described herein include, without limitation, cyclodextrins (e.g., beta-cyclodextrins such as KLEPTOSE®), dimethylsulfoxide (DMSO), sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

In some cases, a composition containing one or more flavonoid compounds provided herein (e.g., one or more flavonoid compounds having the structure of Formula (I) or Formula (II)) can be designed for oral or parenteral (including, without limitation, a subcutaneous, intramuscular, intravenous, intradermal, intra-cerebral, intrathecal, or intraperitoneal (i.p.) injection) administration to a mammal. Compositions suitable for oral administration include, without limitation, liquids, tablets, capsules, pills, powders, gels, and granules. In some cases, compositions suitable for oral administration can be in the form of a food supplement. In some cases, compositions suitable for oral administration can be in the form of a drink supplement. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

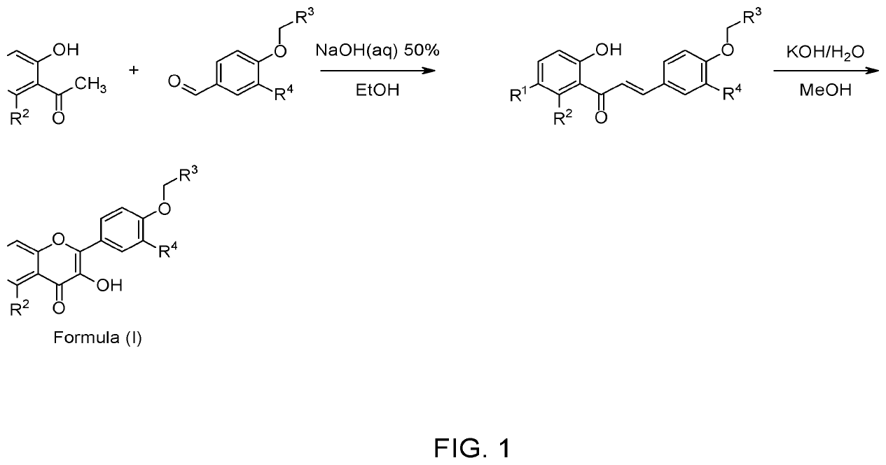

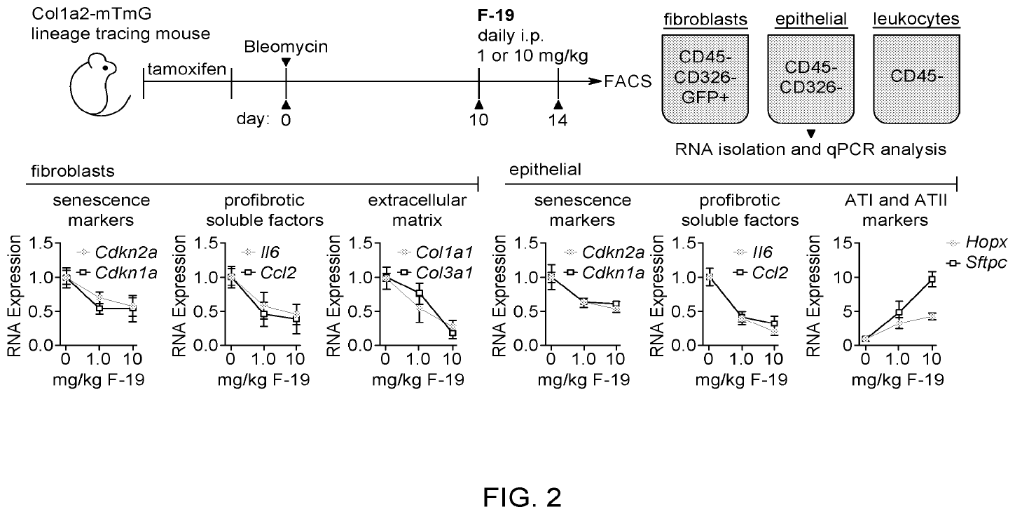

This document also provides methods for making one or more flavonoid compounds described herein (e.g., one or more flavonoid compounds having the structure of Formula (I) or Formula (II). Any appropriate method can be used to make one or more flavonoid compounds provided herein. In some cases, a flavonoid compound having the structure of Formula (I) or Formula (II) can be made as show in FIG. 1. In some cases, a flavonoid compound having the structure of Formula (I) or Formula (II) can be made as described in Example 1.

This document also provides methods for using one or more flavonoid compounds provided herein (e.g., one or more flavonoid compounds having the structure of Formula (I) or Formula (II)). For example, one or more flavonoid compounds having the structure of Formula (I) or Formula (II) can be administered to a mammal (e.g., a human) having one or more fibrotic conditions (e.g., IPF, NASH, PSC, and ocular fibrosis) to treat the mammal. In some cases, a mammal (e.g., a human) having one or more fibrotic conditions (e.g., IPF, NASH, PSC, and ocular fibrosis) can be administered or instructed to self-administer one or more flavonoid compounds having the structure of Formula (I) or Formula (II).

In some cases, one or more (e.g., one, two, three, four, or more) flavonoid compounds provided herein (e.g., one or more flavonoid compounds having the structure of Formula (I) or Formula (II)) can be used to reduce or eliminate of one or more symptoms of one or more fibrotic conditions. For example, a composition including one or more flavonoid compounds having the structure of Formula (I) or Formula (II) can be administered to a mammal (e.g., a human) in need thereof (e.g., a human having one or more fibrotic conditions such as IPF, NASH, PSC, and ocular fibrosis) to reduce or eliminate one or more symptoms of the fibrotic condition (e.g., IPF, NASH, and PSC). Examples of symptoms of IPF disease include, without limitation, shortness of breath (dyspnea), persistent dry cough, tiredness, loss of appetite and weight loss, aching muscles and joints, and clubbing, which is widening and rounding of the tips of the fingers or toes. Examples of symptoms of PSC disease include, without limitation, feeling tired or weak, itchy skin, pain in the abdomen, losing weight without trying, poor appetite, fever, enlarged liver, enlarged spleen, yellow eyes and skin (jaundice), symptoms of cirrhosis and liver failure such as bloating, bruising and bleeding easily, confusion, difficulty thinking or memory loss, redness in the palms of hands, and swelling in legs, ankles or feet. In some cases, one or more (e.g., one, two, three, four, or more) flavonoid compounds provided herein (e.g., one or more flavonoid compounds having the structure of Formula (I) or Formula (II)) can be used to reduce one or more symptoms of fibrotic condition in a mammal having one or more fibrotic conditions by, for example, 10, 20, 30, 40, 50, 60, 70, 80, 90, 95, or more percent.

In some cases, one or more (e.g., one, two, three, four, or more) flavonoid compounds provided herein (e.g., one or more flavonoid compounds having the structure of Formula (I) or Formula (II)) can be used to reduce or eliminate one or more complications associated with a fibrotic condition. For example, a composition including one or more flavonoid compounds having the structure of Formula (I) or Formula (II) can be administered to a mammal (e.g., a human) in need thereof (e.g., a human having one or more fibrotic conditions such as IPF, NASH, PSC, and ocular fibrosis) to reduce or eliminate one or more complications associated with the fibrotic condition. Examples of complications associated with IPF include, without limitation, pulmonary hypertension, acute exacerbation of pulmonary fibrosis, respiratory infection, acute coronary syndrome, thromboembolic disease, adverse medication effects, and lung cancer. Examples of complications associated with PSC include, without limitation, low levels of fat-soluble vitamins, osteoporosis, bile duct infection, portal hypertension, cirrhosis, liver failure, bile duct cancer, gall bladder cancer, colon cancer, and hepatocellular carcinoma. In some cases, one or more (e.g., one, two, three, four, or more) flavonoid compounds provided herein (e.g., one or more flavonoid compounds having the structure of Formula (I)) can be used to reduce one or more complications associated with one or more fibrotic conditions in a mammal having one or more fibrotic conditions by, for example, 10, 20, 30, 40, 50, 60, 70, 80, 90, 95, or more percent.

In some cases, one or more (e.g., one, two, three, four, or more) flavonoid compounds provided herein (e.g., one or more flavonoid compounds having the structure of Formula (I) or Formula (II)) can be used as an anti-fibrotic agent. For example, one or more flavonoid compounds provided herein (e.g., one or more flavonoid compounds having the structure of Formula (I) or Formula (II)) can be used to reduce or eliminate fibrotic scarring in a mammal (e.g., in one or more tissues within a mammal). For example, one or more flavonoid compounds provided herein (e.g., one or more flavonoid compounds having the structure of Formula (I) or Formula (II)) can be used to slow the progression of fibrosis in mammal (e.g., in one or more tissues within a mammal).

In some cases, one or more (e.g., one, two, three, four, or more) flavonoid compounds provided herein (e.g., one or more flavonoid compounds having the structure of Formula (I) or Formula (II)) can be used to reduce or eliminate fibrotic scarring in a mammal. For example, a composition including one or more (e.g., one, two, three, four, or more) flavonoid compounds having the structure of Formula (I) or Formula (II) can be administered to a mammal (e.g., a human) in need thereof (e.g., a human having one or more fibrotic conditions such as IPF, NASH, PSC, and ocular fibrosis) to reduce or eliminate fibrotic scarring in one or more tissues within the mammal. One or more flavonoid compounds provided herein can be used to reduce or eliminate fibrotic scarring in any appropriate tissue within a mammal. Examples of tissues that can have fibrotic scars and that one or more flavonoid compounds provided herein can be used to reduce or eliminate fibrotic scarring in include, without limitation, lung, liver, bile ducts, kidney, heart, and skin. In some cases, one or more (e.g., one, two, three, four, or more) flavonoid compounds provided herein (e.g., one or more flavonoid compounds having the structure of Formula (I) or Formula (II)) can be used to reduce fibrotic scarring in one or more tissues within a mammal having fibrotic scarring by, for example, 10, 20, 30, 40, 50, 60, 70, 80, 90, 95, or more percent.

In some cases, one or more (e.g., one, two, three, four, or more) flavonoid compounds provided herein (e.g., one or more flavonoid compounds having the structure of Formula (I) or Formula (II)) can be used to slow the progression of fibrosis in a mammal. For example, a composition including one or more flavonoid compounds having the structure of Formula (I) or Formula (II) can be administered to a mammal (e.g., a human) in need thereof (e.g., a human having one or more fibrotic conditions such as IPF, NASH, PSC, and ocular fibrosis) to slow the progression of fibrosis in the mammal. One or more flavonoid compounds provided herein can be used to slow the progression of fibrosis in any appropriate tissue within a mammal. Examples of tissues that can be fibrotic and that one or more flavonoid compounds provided herein can be used to slow the progression of fibrosis in include, without limitation, lung, liver, bile ducts, kidney, heart and skin. In some cases, one or more (e.g., one, two, three, four, or more) flavonoid compounds provided herein (e.g., one or more flavonoid compounds having the structure of Formula (I)) can be effective to slow the progression of fibrosis in a mammal having fibrosis by, for example, 10, 20, 30, 40, 50, 60, 70, 80, 90, 95, or more percent. In some cases, one or more (e.g., one, two, three, four, or more) flavonoid compounds provided herein (e.g., one or more flavonoid compounds having the structure of Formula (I)) can be used to slow the progression of fibrosis in a mammal having fibrosis by, for example, at least 6 months (e.g., about 6 months, about 8 months, about 10 months, about 1 year, about 1.5 years, about 2 years, about 2.5 years, about 3 years, about 4 years, about 5 years, or more).

In some cases, one or more (e.g., one, two, three, four, or more) flavonoid compounds provided herein (e.g., one or more flavonoid compounds having the structure of Formula (I) or Formula (II)) can be used as a senolytic agent. For example, one or more flavonoid compounds provided herein (e.g., one or more flavonoid compounds having the structure of Formula (I) or Formula (II)) can be used to induce apoptosis in one or more senescent cells within a mammal. In some cases, one or more flavonoid compounds provided herein (e.g., one or more flavonoid compounds having the structure of Formula (I) or Formula (II)) can exhibit little, or no, ability to induce apoptosis in proliferating cells within a mammal (e.g., a human).

In some cases, one or more (e.g., one, two, three, four, or more) flavonoid compounds provided herein (e.g., one or more flavonoid compounds having the structure of Formula (I) or Formula (II)) can be used to inhibit one or more serine/threonine kinase 17 (STK17) polypeptides. For example, a flavonoid compound having the structure of Formula (I) or Formula (II) can bind to a STK17 polypeptide to inhibit polypeptide function of the STK17 polypeptide. A flavonoid compound having the structure of Formula (I) or Formula (II) can inhibit any appropriate STK17 polypeptide. Examples of STK17 polypeptides that be inhibited by one or more flavonoid compounds provided herein (e.g., one or more flavonoid compounds having the structure of Formula (I) or Formula (II)) include, without limitation, STK17A (DRAK1) polypeptides, and STK17B (DRAK2) polypeptides. In some cases, a flavonoid compound having the structure of Formula (I) or Formula (II) can inhibit a STK17A (DRAK1) polypeptide set forth in any one of National Center for Biotechnology Information (NCBI) GenBank® or GenPept® Accession Nos. 9263 and Q9UEE5. In some cases, a flavonoid compound having the structure of Formula (I) or Formula (II) can inhibit a STK17B (DRAK2) polypeptide set forth in any one of NCBI GenBank® or GenPept® Accession Nos. 9262 and 094768.

In some cases, one or more (e.g., one, two, three, four, or more) flavonoid compounds provided herein (e.g., one or more flavonoid compounds having the structure of Formula (I) or Formula (II)) can be used to inhibit fibroblast activation. A flavonoid compound having the structure of Formula (I) or Formula (II) can inhibit any appropriate fibroblast activation. For example, a flavonoid compound having the structure of Formula (I) or Formula (II) can inhibit TGFβ-induced fibroblast activation.

In some cases, one or more (e.g., one, two, three, four, or more) flavonoid compounds provided herein (e.g., one or more flavonoid compounds having the structure of Formula (I) or Formula (II)) can be used to induce apoptosis of cells (e.g., senescent cells) within a mammal. For example, a composition including one or more flavonoid compounds having the structure of Formula (I) or Formula (II) can be administered to a mammal (e.g., a human) in need thereof (e.g., a human having one or more fibrotic conditions such as IPF, NASH, PSC, and ocular fibrosis) to induce apoptosis in senescent cells within the mammal. One or more flavonoid compounds provided herein can be used to induce apoptosis in any appropriate type of senescent cell within a mammal. Examples of types of cells that can be senescent and that one or more flavonoid compounds provided herein can be used to induce apoptosis in include, without limitation, fibroblasts (e.g., lung fibroblasts) and epithelial cells (e.g., cholangiocytes). For example, one or more flavonoid compounds provided herein (e.g., one or more flavonoid compounds having the structure of Formula (I) or Formula (II)) can be used to reduce the number of senescent cells within a mammal. In some cases, a composition including one or more flavonoid compounds having the structure of Formula (I) or Formula (II) can be administered to a mammal (e.g., a human) in need thereof (e.g., a human having one or more fibrotic conditions such as IPF, NASH, PSC, and ocular fibrosis) to reduce the number senescent cells within the mammal. In some cases, one or more (e.g., one, two, three, four, or more) flavonoid compounds provided herein (e.g., one or more flavonoid compounds having the structure of Formula (I)) can be effective to reduce the number of senescent cells within a mammal having one or more fibrotic conditions by, for example, 10, 20, 30, 40, 50, 60, 70, 80, 90, 95, or more percent.

In some cases, one or more (e.g., one, two, three, four, or more) flavonoid compounds provided herein (e.g., one or more flavonoid compounds having the structure of Formula (I) or Formula (II)) can be used to increase the survival of a mammal (e.g., a human). For example, a composition containing one or more flavonoid compounds having the structure of Formula (I) or Formula (II) can be administered to a mammal (e.g., a human) in need thereof (e.g., a human having one or more fibrotic conditions such as IPF, NASH, PSC, and ocular fibrosis) to increase the survival of the mammal. In some cases, one or more (e.g., one, two, three, four, or more) flavonoid compounds provided herein (e.g., one or more flavonoid compounds having the structure of Formula (I)) can be used to increase the survival of a mammal having one or more fibrotic conditions by, for example, 10, 20, 30, 40, 50, 60, 70, 80, 90, 95, or more percent.

In some cases, one or more (e.g., one, two, three, four, or more) flavonoid compounds provided herein (e.g., one or more flavonoid compounds having the structure of Formula (I) or Formula (II)) can be used to reduce or eliminate inflammation in one or more tissues within a mammal. For example, a composition including one or more flavonoid compounds having the structure of Formula (I) or Formula (II) can be administered to a mammal (e.g., a human) in need thereof (e.g., a human having one or more fibrotic conditions such as IPF, NASH, PSC, and ocular fibrosis) to reduce or eliminate inflammation in one or more tissues within the mammal. One or more flavonoid compounds provided herein can be used to reduce inflammation in any appropriate tissue within a mammal. Examples of tissues that can be inflamed and that one or more flavonoid compounds provided herein can be used to reduce inflammation in include, without limitation, lung, liver, bile ducts, kidney, heart and skin. In some cases, one or more (e.g., one, two, three, four, or more) flavonoid compounds provided herein (e.g., one or more flavonoid compounds having the structure of Formula (I)) can be used to reduce inflammation in one or more tissues within a mammal having a fibrotic condition (e.g., IPF) by, for example, 10, 20, 30, 40, 50, 60, 70, 80, 90, 95, or more percent.

Any appropriate mammal having one or more fibrotic conditions (e.g., IPF, NASH, PSC, and ocular fibrosis) can be treated as described herein (e.g., by administering one or more flavonoid compounds having the structure of Formula (I) or Formula (II)). Examples of mammals that can have one or more fibrotic conditions (e.g., IPF, NASH, PSC, and ocular fibrosis) and can be treated as described herein include, without limitation, humans, non-human primates such as monkeys, dogs, cats, horses, cows, pigs, sheep, mice, and rats. In some cases, a human having one or more fibrotic conditions can be treated by administering one or more flavonoid compounds having the structure of Formula (I) or Formula (II) as described herein.

When treating a mammal (e.g., a human) having one or more fibrotic conditions, the mammal can have any type of fibrotic condition(s). Examples of fibrotic conditions that can be treated as described herein (e.g., by administering one or more flavonoid compounds having the structure of Formula (I) or Formula (II)) include, without limitation, IPF, PSC, NASH, and ocular fibrosis.

In some cases, the methods described herein can include identifying a mammal (e.g., a human) as having one or more fibrotic conditions (e.g., IPF, NASH, PSC, and ocular fibrosis). Any appropriate method can be used to identify a mammal as having one or more fibrotic conditions (e.g., IPF, NASH, PSC, and ocular fibrosis). For example, chest scans including X-ray and high-resolution computed tremography, breathing tests, pulse oximetry, blood test for oxygen and CO₂, exercise capacity, and/or lung biopsy (e.g., to observe signs of scarring) can be used to identify mammals (e.g., humans) having an IPF disease. For example, imaging techniques (e.g., magnetic resonance imaging (MRI), magnetic resonance cholangiopancreatography (MRCP), and endoscopic retrograde cholangiopancreatography (ERCP)), a cholestatic biochemical profile, and/or liver biopsy can be used to identify mammals (e.g., humans) having, a PSC disease.

In some cases, one or more flavonoid compounds provided herein (e. g, one or more flavonoid compounds having the structure of Formula (I) or Formula (II)) can be used to treat a mammal (e.g., a human) having an age-related disease. For example, one or more flavonoid compounds provided herein (e.g., one or more flavonoid compounds having the structure of Formula (I) or Formula (II)) can be administered to a mammal (e.g., a human) having an age-related disease (e.g., to treat the mammal). Examples of age-related diseases that can be treated as described herein (e.g., by administering one or more flavonoid compounds provided herein) include, without limitation, osteoporosis, frailty, cardiovascular diseases, osteoarthritis, pulmonary fibrosis, renal diseases, neurodegenerative diseases, hepatic steatosis, and metabolic dysfunction. In some cases, an age-related disease that can be treated as described herein can be as described elsewhere (see, e.g., Kaur et al., Transl. Res., 226:96-104 (2020)). A composition containing one or more flavonoid compounds provided herein (e.g., one or more flavonoid compounds having the structure of Formula (I) or Formula (II)) can be administered to a mammal (e.g., a human) having one or more fibrotic conditions (e.g., IPF, NASH, PSC, and ocular fibrosis) in any appropriate amount (e.g., any appropriate dose). An effective amount of a composition containing one or more flavonoid compounds having the structure of Formula (I) or Formula (II) can be any amount that can treat a mammal (e.g., a mammal having one or more fibrotic conditions such as IPF, NASH, PSC, and ocular fibrosis) as described herein without producing significant toxicity to the mammal. In some cases, an effective amount of one or more flavonoid compounds having the structure of Formula (I) or Formula (II) can be from about 0.1 μM to about 100 μM (e.g., from about 0.1 μM to about 75 μM, from about 0.1 μM to about 60 μM, from about 0.1 μM to about 50 μM, from about 0.1 μM to about 40 μM, from about 0.1 μM to about 30 μM, from about 0.1 μM to about 20 μM, from about 0.1 μM to about 10 μM, from about 0.1 μM to about 1 μM, from about 10 μM to about 100 μM, from about 20 μM to about 100 μM, from about 30 μM to about 100 μM, from about 40 μM to about 100 μM, from about 50 μM to about 100 μM, from about 60 μM to about 100 μM, from about 75 μM to about 100 μM, from about 1 μM to about 75 μM, from about 10 μM to about 50 μM, from about 20 μM to about 40 μM, from about 1 μM to about 30 μM, from about 30 μM to about 50 μM, from about 40 μM to about 60 μM, from about 50 μM to about 70 μM, from about 60 μM to about 80 μM, or about 30 μM). In some cases, an effective amount of one or more flavonoid compounds having the structure of Formula (I) or Formula (II) can be from about 200 nM IC₅₀ to about 800 nM IC₅₀ (e.g., from about 200 nM IC₅₀ to about 700 nM IC₅₀, from about 200 nM IC₅₀ to about 600 nM IC₅₀, from about 200 nM IC₅₀ to about 500 nM IC₅₀, from about 200 nM IC₅₀ to about 400 nM IC₅₀, from about 200 nM IC₅₀ to about 300 nM IC₅₀, from about 300 nM IC₅₀ to about 800 nM IC₅₀, from about 400 nM IC₅₀ to about 800 nM IC₅₀, from about 500 nM IC₅₀ to about 800 nM IC₅₀, from about 600 nM IC₅₀ to about 800 nM IC₅₀, from about 700 nM IC₅₀ to about 800 nM IC₅₀, from about 300 nM IC₅₀ to about 700 nM IC₅₀, from about 400 nM IC₅₀ to about 600 nM IC₅₀, from about 300 nM IC₅₀ to about 400 nM IC₅₀, from about 400 nM IC₅₀ to about 500 nM IC₅₀, from about 500 nM IC₅₀ to about 600 nM IC₅₀, from about 600 nM IC₅₀ to about 700 nM IC₅₀, about 200 nM IC₅₀, or about 800 nM IC₅₀). In some cases, an effective amount of one or more flavonoid compounds having the structure of Formula (I) or Formula (II) can include from about 0.1 milligrams per kilogram body weight (mg/kg) to about 200 mg/kg (e.g., from about 0.1 mg/kg to about 175 mg/kg, from about 0.1 mg/kg to about 150 mg/kg, from about 0.1 mg/kg to about 125 mg/kg, from about 0.1 mg/kg to about 100 mg/kg, from about 0.1 mg/kg to about 75 mg/kg, from about 0.1 mg/kg to about 50 mg/kg, from about 0.1 mg/kg to about 25 mg/kg, from about 0.1 mg/kg to about 10 mg/kg, from about 0.1 mg/kg to about 5 mg/kg, from about 1 mg/kg to about 200 mg/kg, from about 10 mg/kg to about 200 mg/kg, from about 25 mg/kg to about 200 mg/kg, from about 50 mg/kg to about 200 mg/kg, from about 75 mg/kg to about 200 mg/kg, from about 100 mg/kg to about 200 mg/kg, from about 150 mg/kg to about 200 mg/kg, from about 1 mg/kg to about 150 mg/kg, from about 10 mg/kg to about 100 mg/kg, from about 25 mg/kg to about 75 mg/kg, from about 1 mg/kg to about 5 mg/kg, from about 5 mg/kg to about 10 mg/kg, from about 10 mg/kg to about 25 mg/kg, from about 25 mg/kg to about 50 mg/kg, from about 50 mg/kg to about 100 mg/kg, from about 100 mg/kg to about 150 mg/kg, or about 2 mg/kg) F-4N. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the fibrotic condition (e.g., IPF, NASH, and PSC) in the mammal being treated may require an increase or decrease in the actual effective amount administered.

A composition containing one or more flavonoid compounds provided herein (e.g., one or more flavonoid compounds having the structure of Formula (I) or Formula (II)) can be administered to a mammal (e.g., a mammal having one or more fibrotic conditions such as IPF, NASH, PSC, and ocular fibrosis) in any appropriate frequency. The frequency of administration can be any frequency that can treat a mammal (e.g., a mammal having one or more fibrotic conditions such as IPF, NASH, PSC, and ocular fibrosis) without producing significant toxicity to the mammal (e.g., human). For example, the frequency of administration can be from about once a day to about once a week, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition containing one or more flavonoid compounds provided herein (e.g., one or more flavonoid compounds having the structure of Formula (I) or Formula (II)) can be administered to a mammal (e.g., a mammal having one or more fibrotic conditions such as IPF, NASH, PSC, and ocular fibrosis) for any appropriate duration. An effective duration for administering or using a composition containing one or more flavonoid compounds having the structure of Formula (I) or Formula (II) can be any duration that can treat a mammal (e.g., a mammal having one or more fibrotic conditions such as IPF, NASH, PSC, and ocular fibrosis) without producing significant toxicity to the mammal (e.g., a human). For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, the methods for treating a mammal (e.g., a mammal such as a human having one or more fibrotic conditions such as IPF, NASH, PSC, and ocular fibrosis) as described herein (e.g., by administering one or more flavonoid compounds having the structure of Formula (I) or Formula (II)) can include administering to the mammal one or more flavonoid compounds having the structure of Formula (I) or Formula (II) as the sole active ingredient(s) to treat the mammal. For example, a composition containing one or more flavonoid compounds having the structure of Formula (I) or Formula (II) can include the flavonoid compound(s) having the structure of Formula (I) or Formula (II) as the sole active ingredient in the composition that is effective to treat a mammal (e.g., a mammal having one or more fibrotic conditions such as IPF, NASH, PSC, and ocular fibrosis).

In some cases, the methods for treating a mammal (e.g., a mammal such as a human having one or more fibrotic conditions such as IPF, NASH, PSC, and ocular fibrosis) as described herein (e.g., by administering one or more flavonoid compounds having the structure of Formula (I) or Formula (II)) also can include administering to the mammal one or more (e.g., one, two, three, four, five or more) additional agents/therapies used to treat a condition (e.g., one or more fibrotic conditions such as IPF, PSC, and/or ocular fibrosis). For example, a combination therapy used to treat one or more fibrotic conditions (e.g., IPF, NASH, PSC, and ocular fibrosis) can include administering to the mammal (e.g., a human) one or more flavonoid compounds having the structure of Formula (I) or Formula (II) described herein and one or more (e.g., one, two, three, four, five or more) agents used to treat one or more fibrotic conditions (e.g., IPF, NASH, PSC, and ocular fibrosis). Examples of agents that can be administered to a mammal to treat IPF disease include, without limitation, pirfenidone, nintedanib, N-acetylcysteine, phosphodiesterase inhibitors (e.g., sildenafil, vardenafil, tadalafil, and avanafil), lysophosphatidic acid receptor antagonists (e.g., promethazine, FTY720, AM152, BMS-986020, VPC 12249, AM966, and AM095), autotaxin inhibitors (e.g., taribavirin, BI-2545, GLPG1690, BBT 877, SAR100842, and BMS-986,020), D1 receptor agonists (e.g., minaprine, dopamine, levodopa, apomorphine, fenoldopam, pergolide, bromocriptine, and cabergoline), dasatinib, rho kinase inhibitors (e.g., hydroxyfasudil, ripasudil, netarsudil, and belumosudil), IL-13 neutralizing antibodies (e.g., lebrikizumab, tralokinumab, and dupilumab), CTGF neutralizing antibodies (e.g., pamrevlumab), and any combinations thereof. Examples of agents that can be administered to a mammal to treat PSC disease include, without limitation, ursodeoxycholic acid (UDCA), corticosteroids (e.g., glucocorticoids such as prednisolone), bile acid sequestrants, antibiotics, antihistamines, and any combinations thereof.

In cases where one or more flavonoid compounds provided herein (e.g., one or more flavonoid compounds having the structure of Formula (I) or Formula (II)) are used in combination with additional agents used to treat one or more fibrotic conditions, the one or more additional agents can be administered at the same time (e.g., in a single composition containing both one or more flavonoid compounds having the structure of Formula (I) or Formula (II) and the one or more additional agents) or independently. For example, one or more flavonoid compounds having the structure of Formula (I) or Formula (II) described herein can be administered first, and the one or more additional agents administered second, or vice versa.

In some cases, the methods for treating a mammal (e.g., a mammal such as a human having one or more fibrotic conditions such as IPF, NASH, PSC, and ocular fibrosis) as described herein (e.g., by administering one or more flavonoid compounds having the structure of Formula (I) or Formula (II)) also can include performing one or more (e.g., one, two, three, four, five or more) additional therapies used to treat one or more fibrotic conditions (e.g., IPF, NASH, PSC, and ocular fibrosis) on the mammal. Examples of therapies used to treat the IPF disease include, without limitation, oxygen therapy, pulmonary rehabilitation, and/or lung transplantation. Examples of therapies used to treat the PSC disease include, without limitation, endoscopic therapy (e.g., balloon dilatation and stent placement), percutaneous therapy, non-transplant surgery, liver transplantation, and/or fecal microbiome transplant.

In cases where one or more flavonoid compounds provided herein (e.g., one or more flavonoid compounds having the structure of Formula (I) or Formula (II)) are used in combination with one or more additional therapies used to treat one or more fibrotic conditions (e.g., IPF, NASH, PSC, and ocular fibrosis), the one or more additional therapies can be performed at the same time or independently of the administration of one or more flavonoid compounds having the structure of Formula (I) or Formula (II) described herein. For example, one or more flavonoid compounds having the structure of Formula (I) or Formula (II) described herein can be administered before, during, or after the one or more additional therapies are performed.

The invention will be further described in the following examples, which do not limit the scope of the invention described in the claims.

### Examples

### Example 1: Design and Synthesis of Flavonoid Compounds

This Example describes the design and characterization of flavonoid compounds that can be used to treat one or more fibrotic conditions.

### Methods

### Compound Synthesis

Equimolar amounts (2-8 mmol) of the hydroxyacetophenone was combined with the aldehyde in 10-50 mL of EtOH. 5-12 mL of 50% NaOH(aq) was added to the reaction and allowed to proceed for 6-48 hours at room temperature while monitoring by thin layer chromatography (TLC). After aldehyde depletion the resulting calchone was precipitated by 10% HCl (aq). Calchone was isolated, dried, weighed, and dissolved (1-4 mmol) in 8-30 mL of MeOH containing 1M KOH. 5-15 mL of H₂O₂ was added to the reaction and allowed to proceed for 1-6 hours at room temperature while monitoring by TLC. Final product was precipitated by 10% HCl (aq) and purified by recrystallization. Structure and purity (>95%) were confirmed by proton nuclear magnetic resonance.

A schematic of a synthesis of exemplary flavonoid compounds provided herein is shown in FIG. 1.

### Apoptosis

Human adult lung fibroblasts were replicatively passaged until senescence (confirmed by RNA and senescence associated beta galactosidase staining). In a parallel experiment, low passage proliferating fibroblasts from the same donor were compared. Cells were plated into 96-well plates and treated with the indicated compound (6-point dose-response) and incubated for 96 hours. Cells were then fixed using 4% PFA, permeabilized using 0.25% triton x-100, and nuclear stained using a primary antibody for cleaved caspase-3, secondary fluorescence conjugated antibody, and DAPI. Cells were then imaged using a 4× objective on a Cytation5 microscope and cell counts were quantified using automated software (Biotek Gen5). Data shown are the EC₅₀ values calculated for each compounds efficacy to induce apoptosis in senescence vs. proliferating lung fibroblasts, from the calculated dose-response curves, N=3 independent experiments.

### Fibroblast Activation

Human adult lung fibroblasts (passage 3) were plated into 96-well plates and treated with the indicated compound (6-point dose-response) +2 ng/mL TGFβ to stimulated fibroblast activation. Cells were incubated for 96 hours, fixed using 4% PFA, permeabilized using 0.25% triton x-100, and stained using a primary antibody recognizing alpha-smooth muscle actin and a secondary fluorescent conjugated antibody, and DAPI. Cells were then imaged using a 4× objective on a Cytation5 microscope and alpha-smooth muscle actin intensity was quantified using automated software (Biotek Gen5). Data shown are the IC₅₀ values for each compounds efficacy at reducing expression of alpha-smooth muscle actin, calculated from the calculated dose-response curves, N=3 independent.

### Results

Flavonoid compounds were evaluated for mechanisms of therapeutic efficacy using high throughput assays. Characteristics of exemplary flavonoid compounds are summarized in Table 1.

TABLE 1

| | | | | | Senescent | Proliferating | Fibroblast |
|---|---|---|---|---|---|---|---|
| | | | | | fibroblast | fibroblast | activation |
| | | | | | apoptosis EC50 | apoptosis EC50 | (EC50) |
| Name | R1 | R2 | R3 | R4 | (μM) | (μM) | (μM) |
| F-19 | H | H | CH₃ | H | 2.4 | 24.5 | 2.4 |
| F-20 | H | H | phenyl | H | 1.5 | 27.6 | 2.0 |
| F—2N | H | H | 2-pyridine | H | inactive | inactive | inactive |
| F—3N | H | H | 3-pyridine | H | 44.1 | inactive | inactive |
| F—4N | H | H | 4-pyridine | H | 0.8 | inactive | 1.8 |
| 6-MeOH—F-19 | H | MeOH | CH₃ | H | 2.7 | 15.1 | 4.0 |
| 6-MeOH—F-20 | H | MeOH | phenyl | H | 0.8 | 5.1 | 2.0 |
| F-19(Me) | H | H | CH₂CH₃ | H | 3.4 | 19.5 | 4.0 |
| F-19(3MeOH) | H | H | CH₃ | MeOH | 5.9 | 10.5 | 6.4 |
| 3Ethoxy | H | H | H | Ethoxy | inactive | inactive | inactive |
| F—CP | H | H | cyclopropyl | H | 0.9 | 10.9 | 1.1 |
| 5-MeOH—F-20 | MeOH | H | phenyl | H | 0.35 | 5.5 | 0.9 |
| 5-MeOH—F—4N | MeOH | H | 4-pyridine | H | inactive | inactive | 2.1 |

### Example 2: F-4N and Lung Fibrosis

This Example describes the use of one or more flavonoid compounds having the structure of Formula (I) in treating lung fibrosis.

### Methods

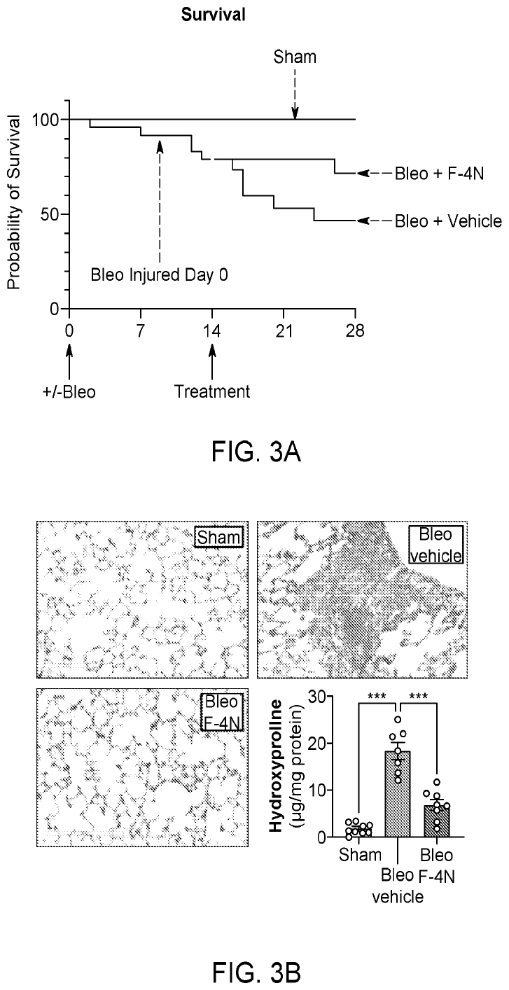

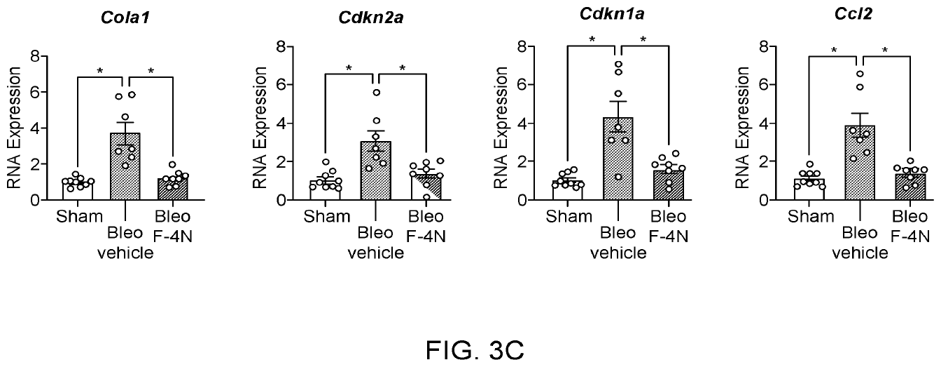

FIG. 3. Lung fibrosis was induced in 10-18-month-old C₅₇/B6 mice (average ˜15-months) using intratracheal bleomycin (1.1 units/kg) on day 0 of the study. On day 14 one group was administered F-4N (10 mg/kg daily i.p.) for 14 days prior to harvesting the lung for outcomes and senescence clearance and fibrosis. Lung architecture and fibrosis were observed by trichome histological staining and hydroxyproline content. Hydroxyproline content was measured using a hydroxyproline assay kit (Biovision) according to the manufacturer's instructions with slight modification. The lung tissues were weighed, homogenized in sterile water (10 mg of tissue per 100 μL H₂O) and hydrolyzed in 12 M HCl in a pressure-tight, Teflon capped vial at 120° C. for 3 hours followed by filtration through a 45 μm Spin-X Centrifuge Tube filter (Corning). Ten μL of the hydrolyzed samples was dried in a Speed-Vac for 2 hours, followed by incubation with 100 μL of Chloramine T reagent for 5 minutes at room temperature and 100 μL of 4-(Dimethylamino)benzaldehyde (DMAB) for 90 minutes at 60° C. The absorbance of oxidized hydroxyproline was determined at 560 nm. Hydroxyproline concentrations were calculated from a standard curve generated using known concentrations of trans-4-hydroxyl-L-proline. The total amount of protein isolated from the weighed tissues was determined by using a protein assay kit (Bio-Rad, absorbance at 595 nm). The amount of collagen was expressed in μg/mg total protein. qPCR: RNA isolation using RNeasy Plus Mini Kit (Qiagen) according to the manufacturer's instructions. Isolated RNA (250 ng) was then used to synthesize cDNA using SuperScript VILO (Invitrogen). Quantitative PCR was performed using FastStart Essential DNA Green Master (Roche) and analyzed using a LightCycler 96 (Roche). Data are expressed as a fold change by ΔΔCt relative to glyceraldehyde-3-phosphate dehydrogenase (GAPDH).

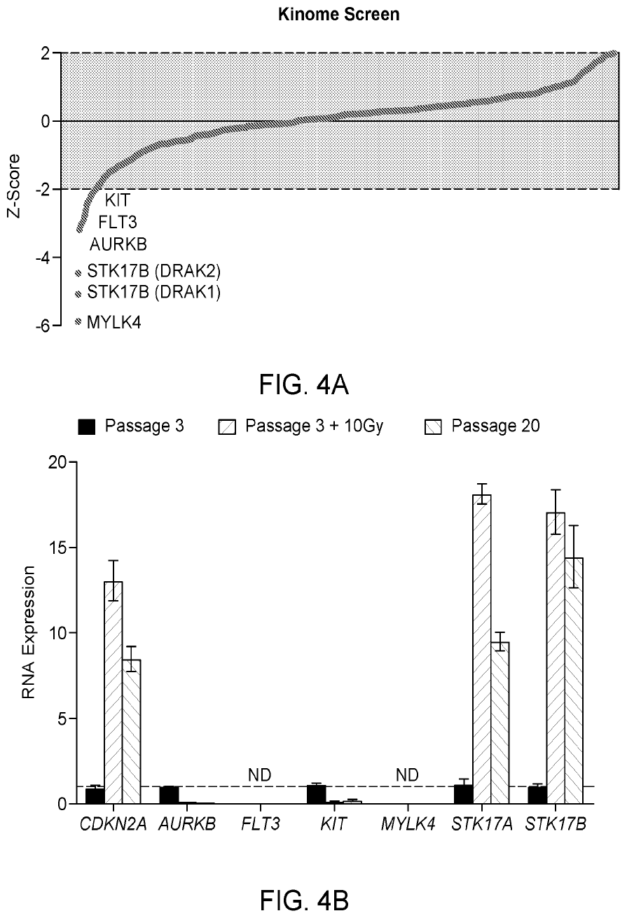

FIG. 4A. F-4N was sent to Reaction Biology to be tested against their entire wild-type kinase panel. 1 μM F-4N was tested in an ATP competition assay against 420+ kinases. Z-scores were calculated to identify true hits over noise.

FIG. 4B. Expression of CDKN2A (senescence marker) was compared to the putative targets identified in the kinome screen by qPCR in low passage fibroblasts compared to senescent fibroblasts generated by radiation (10Gy) or replicative passage.

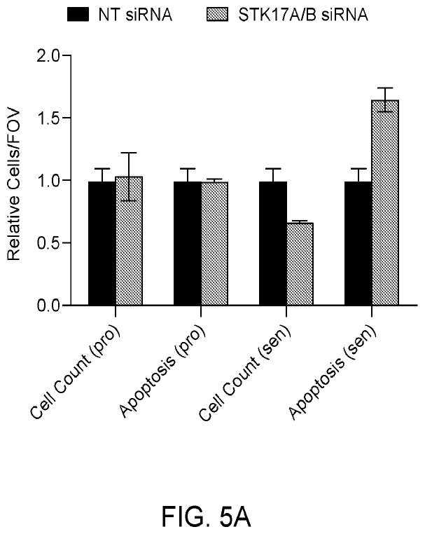

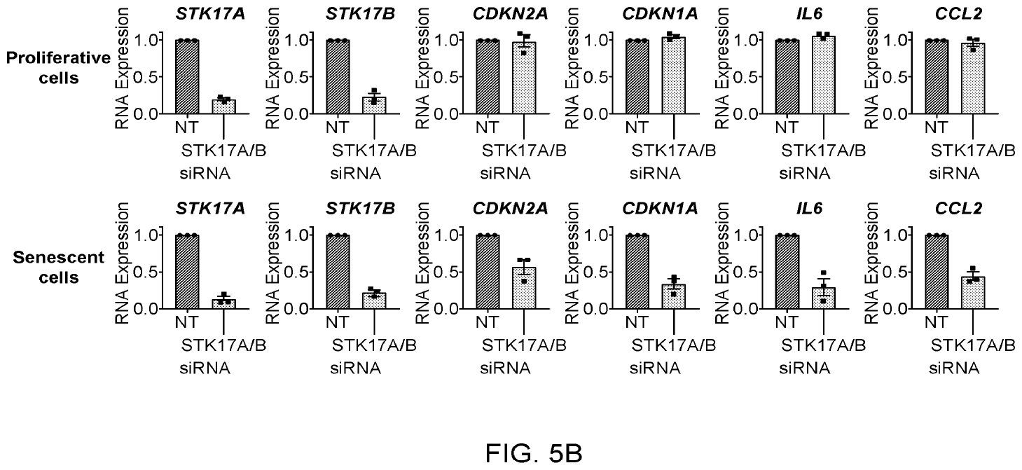

FIG. 5A. Cell counts and apoptosis were compared in proliferating low-passage fibroblasts (pro) vs. senescent high-passage fibroblasts (sen) following siRNA transfection targeting the putative molecular targets of F-4N-STK-17A and STK17B. Cell counts and apoptosis were determined by cleaved caspase-3 and DAPI staining and automated microscopy.

FIG. 5B. qPCR expression of STK17A, STK17B, and senescence markers were compared in proliferating low-passage fibroblasts vs. senescent high-passage fibroblasts following siRNA transfection targeting the putative molecular targets of F-4N-STK-17A and STK17B.

### Results

### F-4N and Lung Fibrosis

Mice that were administered F-4N exhibited improved survival following bleomycin-induced fibrosis (FIG. 3A). Aged mice exhibit chronic fibrosis following one-time administration of bleomycin. Improved survival strongly suggests a beneficial impact of F-4N.

The mice that were administered F-4N also demonstrated less fibrosis and improved lung architecture assessed by trichome histological staining and reduced hydroxyproline content as compared to mice that did not receive any F-4N (FIG. 3B). These results suggest F-4N is improving lung fibrosis in this chronic model.

Lung tissue was examined for expression of Collagen I and of senescence markers. Mice that were administered F-4N had reduced expression of Collagen I and reduced expression of senescence markers (FIG. 3C). These in vivo results are consistent with our in vitro data and overall hypothesis that F-4N has a dual impact on lung fibrosis-clearance of senescent cells and blockage of fibroblast activation.

### Mechanism of Action

Kinome screening was used to test F-4N (1 μM) against ˜400 kinases. Putative F-4N targets identified by kinome screening include STK17A (also known as DRAK1), STK17B (also known as DRAK2), and MYLK4, AURKB, FLT3, and KIT (FIG. 4A). Expression of the putative molecular targets of F-4N were measured and STK17A and STK17B were found to be highly overexpressed in senescent fibroblasts compared to low passage fibroblasts.

### F-4N and Lung Senescence

To determine if the putative F-4N targets could regulate senescence, apoptosis was evaluated in lung fibroblasts (senescence and non-senescent) that were treated with siRNA targeting STK17A and STK17B (FIG. 5A). Expression of senescence markers was also evaluated in proliferative and senescent cells that were treated with siRNA targeting STK17A polypeptides and STK17B polypeptides (FIG. 5B). These data further support F-4N can mediate senolytic activity through inhibition of STK17A/B (DRAK1/2).

### Example 3: F-4N and PSC

This Example describes the use of one or more flavonoid compounds having the structure of Formula (I) in treating PSC.

### Methods

### Hydroxyproline Assay

Hydroxyproline content was measured using a hydroxyproline assay kit (Biovision) according to the manufacturer's instructions with slight modification. The liver tissues were weighed, homogenized in sterile water (10 mg of tissue per 100 μL H₂O) and hydrolyzed in 12 M HCl in a pressure-tight, Teflon capped vial at 120° C. for 3 hours followed by filtration through a 45 μm Spin-X Centrifuge Tube filter (Corning). Ten μL of the hydrolyzed samples was dried in a Speed-Vac for 2 hours, followed by incubation with 100 μL of Chloramine T reagent for 5 minutes at room temperature and 100 μL of 4-(dimethylamino)benzaldehyde (DMAB) for 90 minutes at 60° C. The absorbance of oxidized hydroxyproline was determined at 560 nm. Hydroxyproline concentrations were calculated from a standard curve generated using known concentrations of trans-4-hydroxyl-L-proline. The total amount of protein isolated from the weighed tissues was determined by using a protein assay kit (Bio-Rad, absorbance at 595 nm). The amount of collagen was expressed in μg/mg total protein.

### Picrosirius Staining

Paraffin-embedded liver sections were de-paraffinized and re-hydrated by heating at 60° C. for 10-30 minutes, then passaging in the following solutions:

$$Xylene 1 - 2 \times 10 minutes$$

$$100 \% EtOH - 1 \times 2 minutes ; 95 \% EtOH - 1 minute ;$$

$$70 \% EtOH - 1 minute ; 50 \% EtOH - 1 minute$$

$$PBS - 3 \times 3 minutes$$

The slides were then stained with a solution containing 0.1% picrosirius red (Direct Red 80) and 0.1% fast green (counterstain) in saturated aqueous solution of picric acid, for 60 minutes at room temperature, followed by one wash in distilled water, de-hydration in 3 changes of 100% EtOH, clear in xylene and mount in resinous medium.

### Liver Function Tests

Serum alanine aminotransferase (ALT), alkaline phosphatase (ALP) and total bile acids were measured using a commercially available veterinary chemistry analyzer (VetScan 2, Abaxis).

### Reverse Transcription-Quantitative Polymerase Chain Reaction

RNA isolation using RNeasy Plus Mini Kit (Qiagen) according to the manufacturer's instructions. Isolated RNA (250 ng) was then used to synthesize cDNA using SuperScript VILO (Invitrogen). Quantitative PCR was performed using FastStart Essential DNA Green Master (Roche) and analyzed using a LightCycler 96 (Roche). Data are expressed as a fold change by ΔΔCt relative to glyceraldehyde-3-phosphate dehydrogenase (GAPDH).

### Results

### F-4N and Liver Fibrosis

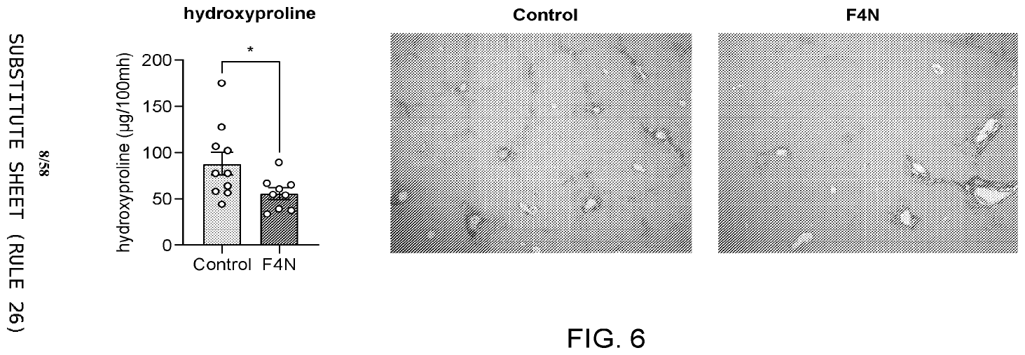

Mice that were administered F-4N demonstrated less collagen fiber deposition, especially in the portal-to-portal areas, and reduced hydroxyproline content as compared to mice that did not receive any F-4N (FIG. 6). These results suggest F-4N reduces established liver fibrosis in a mouse model of PSC.

### F-4N and Liver Function

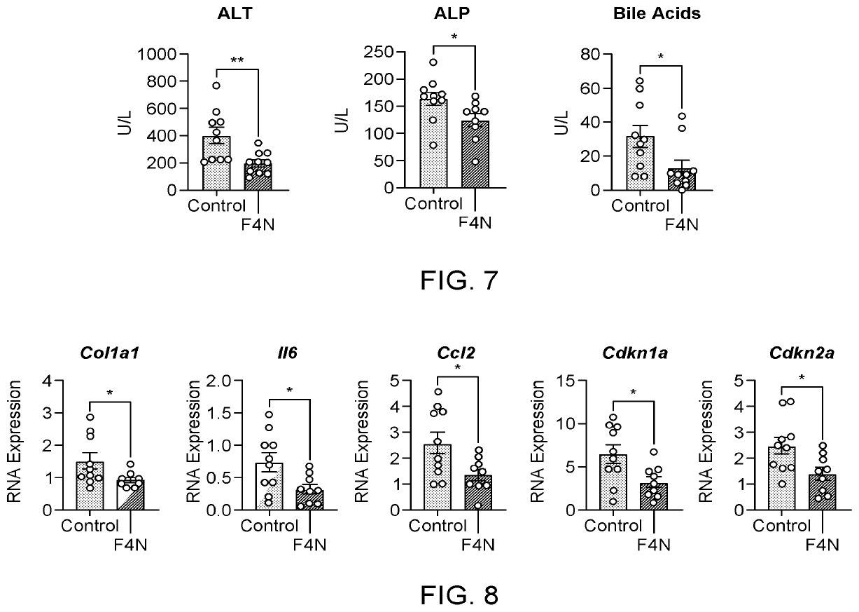

Livers from mice were examined for expression of liver function markers. Mice that were administered F-4N demonstrated lower levels of ALT, ALP and bile acids as compared to mice that did not receive any F-4N (FIG. 7). These results suggest F-4N ameliorates liver injury and cholestasis in a mouse model of PSC.

### F-4N and Liver Senescence

Liver tissue was examined for expression of Collagen I, inflammatory, and senescence markers. Mice that were administered F-4N had reduced expression of Collagen I and reduced expression of inflammatory and senescence markers (FIG. 8). These results suggest F-4N effectively targets senescent cells, resulting in reduced inflammation and liver fibrosis in a mouse model of PSC.

### Example 4: Flavonol Structure Optimization

### Materials and Methods

For senescent fibroblast survival experiments, human adult lung fibroblasts were replicatively passaged until senescence (confirmed by RNA and senescence associated beta galactosidase staining). Cells were then plated into 96-well plates and treated with the indicated compound and incubated for 96 hours. Cells were then fixed using 4% PFA, permeabilized using 0.25% triton x-100, and nuclear stained using DAPI. Cells were then imaged using a 4× objective on a Cytation5 microscope and cell counts were quantified using automated software (Biotek Gen5). Data shown are plotted as % survival, normalized to the vehicle treated well. Mean+/−SEM, N=3 independent experiments. For the TGFβ stimulated collagen deposition experiments (square datapoints), human adult lung fibroblasts (passage 3) were plated into 96-well plates and treated with the indicated compound +2 ng/mL TGFβ to stimulated collagen expression. Cells were incubated for 96 hours, fixed using 4% PFA, permeabilized using 0.25% triton x-100, and stained using a primary antibody recognizing type I collagen and a secondary infrared conjugated antibody. Wells were then imaged at 1× using a Odyssey Lx (LI-CORE) infrared imager and collagen intensity was quantified using automated software. Data shown are plotted as % collagen intensity, normalized to the vehicle treated well. Mean+/−SEM, N=3 independent experiments.

### Results

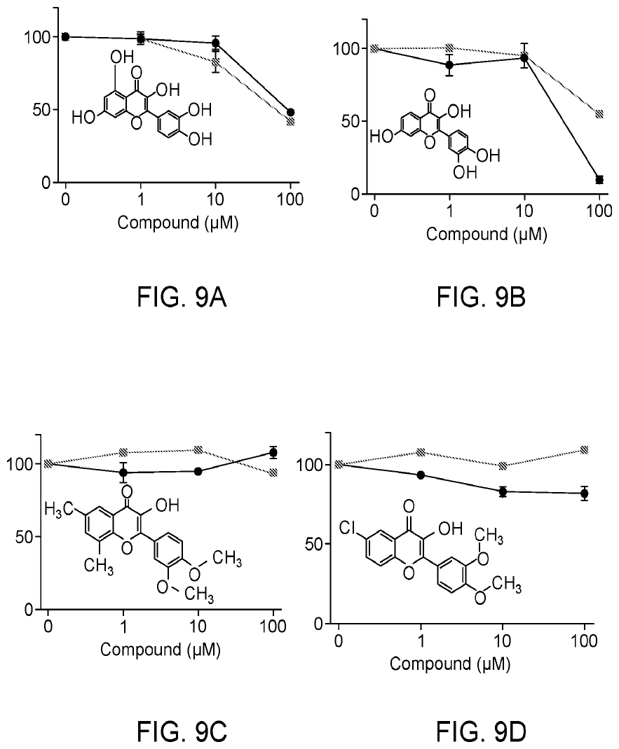

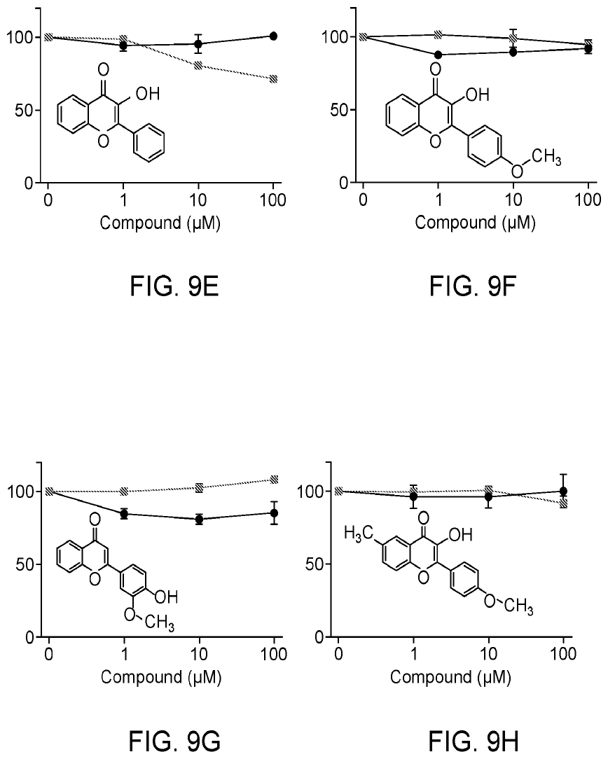

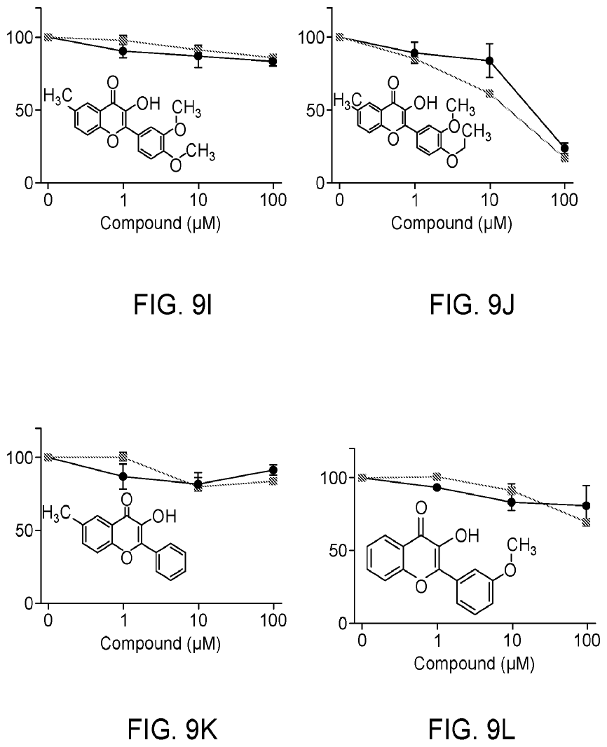

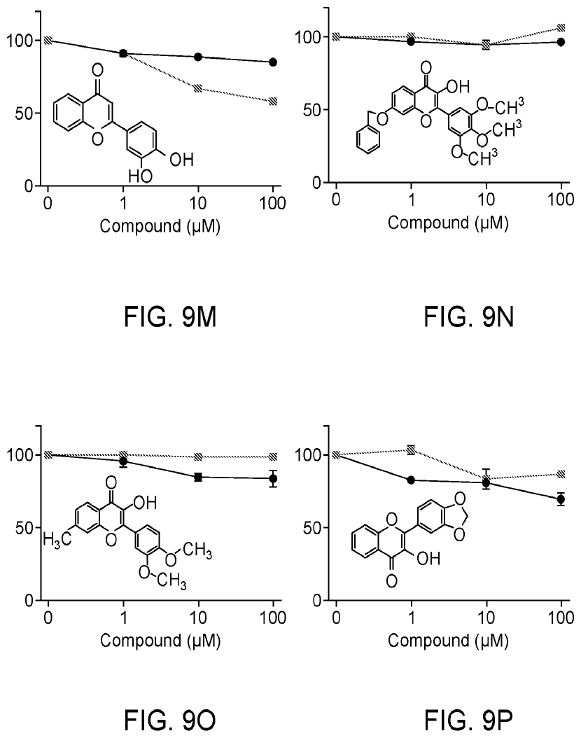

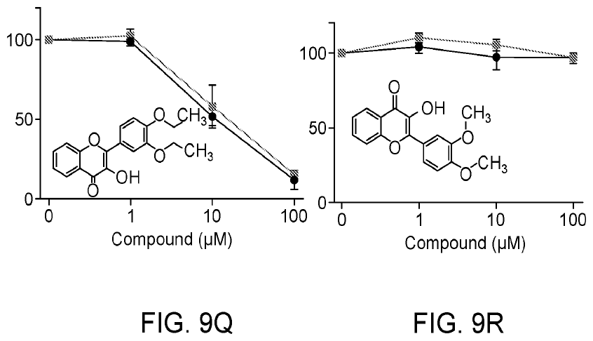

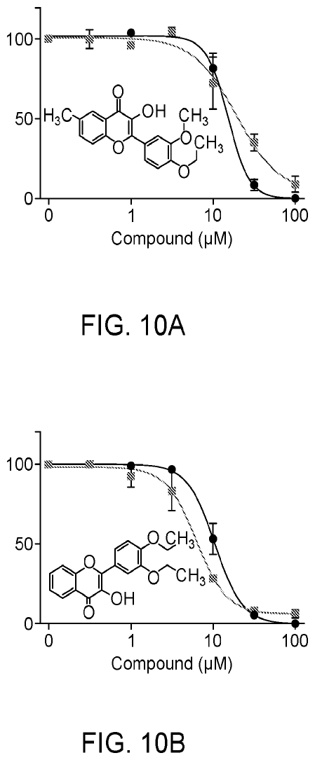

Flavonols were evaluated for the ability to differentially induce cell death in senescent fibroblasts and also for the ability to block collagen deposition in TGFβ stimulated low passage fibroblasts in a 3 dose response curve (FIG. 9). Both 2-(4-ethoxy-3-methoxyphenyl)-3-hydroxy-6-methylchromen-4-one and 2-(3,4-diethoxyphenyl)-3-hydroxy-4h-1-benzopyran-4-one demonstrated effective toxicity against senescent cells and blocked collagen deposition. This was confirmed in a 6-point dose response (FIG. 10). Both compounds contain a para-position ethoxy modification on the B ring of the flavonol core.

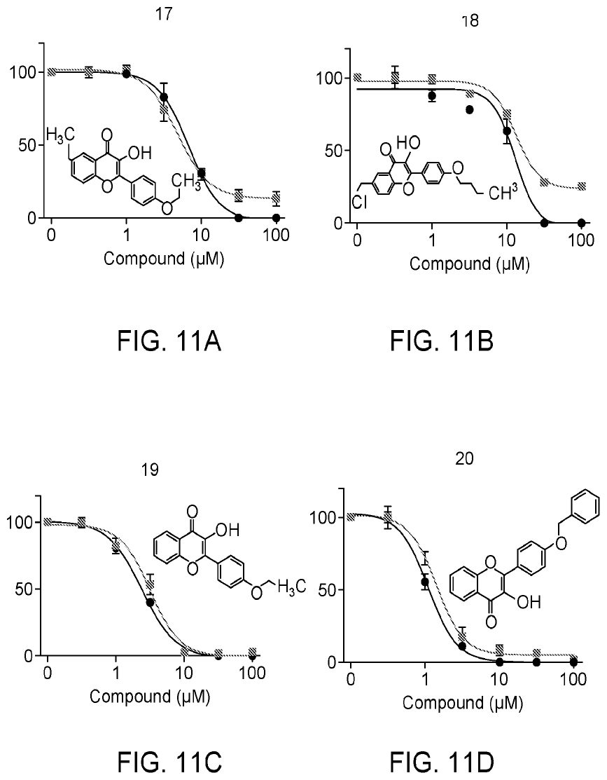

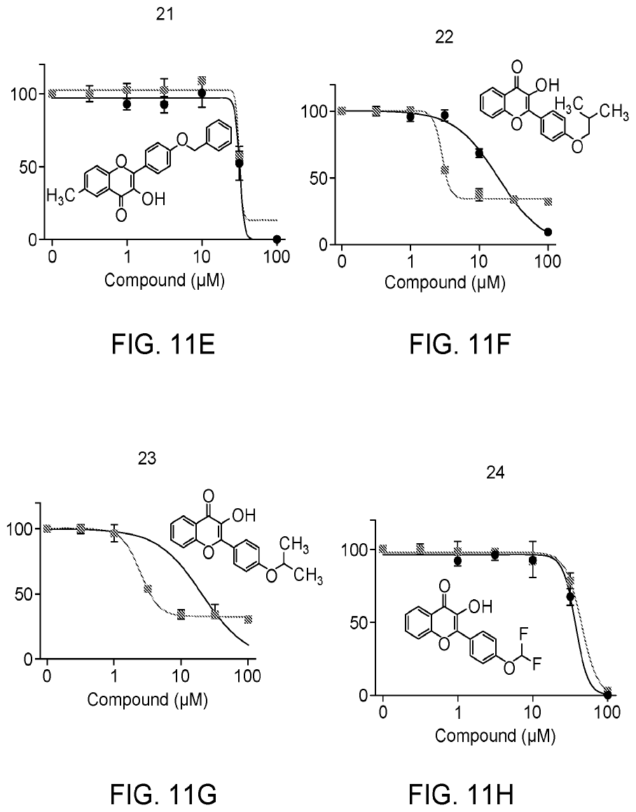

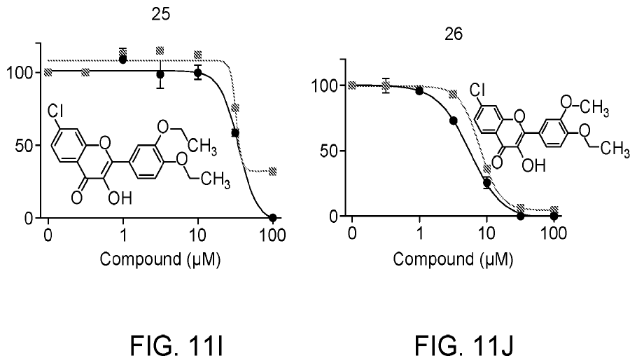

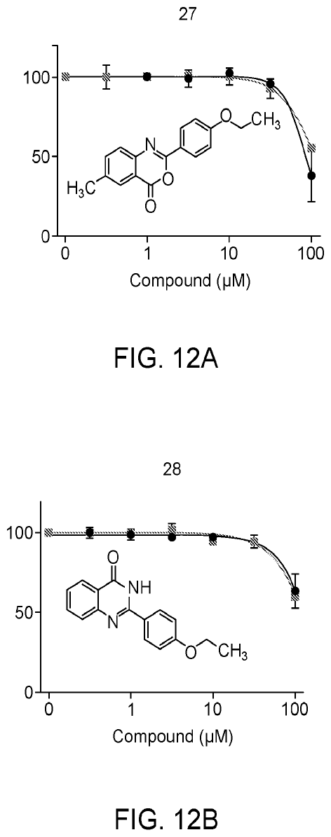

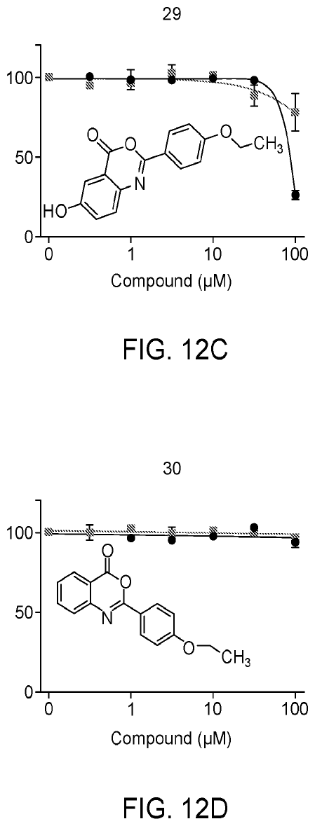

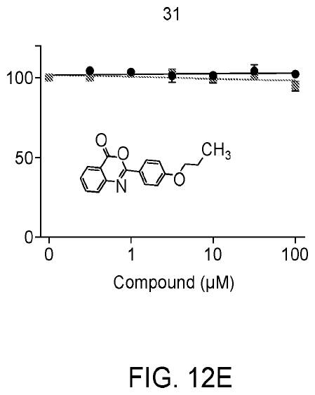

A second generation of flavonol compounds focused on this para ethoxy modification on the B ring of the flavonol core, were evaluated for their impact on reducing senescence fibroblast survival and blockade of collagen deposition in non-senescence fibroblasts stimulated with TGFβ. (FIG. 11). Based on these results we identified a structure activity relationship where para ethoxy modifications to the B ring drives potency, and unsubstituted A rings are optimal. Novel synthetic compounds explored in Table 1, including molecule F-4N were designed based on this SAR.

Finally, flavonols containing heteroatom substitutions within the aromatic core were tested for the impact on senescence fibroblast survival and blockage of collagen deposition. Although these analogs contained the para ethoxy in a position consistent with a flavonoid B ring, they displayed a significant drop in potency.

### Example 5: Treating IPF

A human identified as having IPF is administered or self-administers a composition including one or more flavonoid compounds having the structure of Formula (I). In some cases, the administered flavonoid compound(s) can reduce the severity of one or more symptoms of IPF. In some cases, the administered flavonoid compound(s) can reduce the amount of fibrotic scarring in the lung(s) of the human.

### Example 6: Treating PSC

A human identified as having PSC is administered or self-administers a composition including one or more flavonoid compounds having the structure of Formula (I). In some cases, the administered flavonoid compound(s) can reduce the severity of one or more symptoms of PSC. In some cases, the administered flavonoid compound(s) can reduce the amount of fibrotic scarring in the liver of the human.

### Example 7: F-4N and Lung Fibrosis

The results in this Example re-present and expand on at least some of the results provided in other Examples.

### Methods

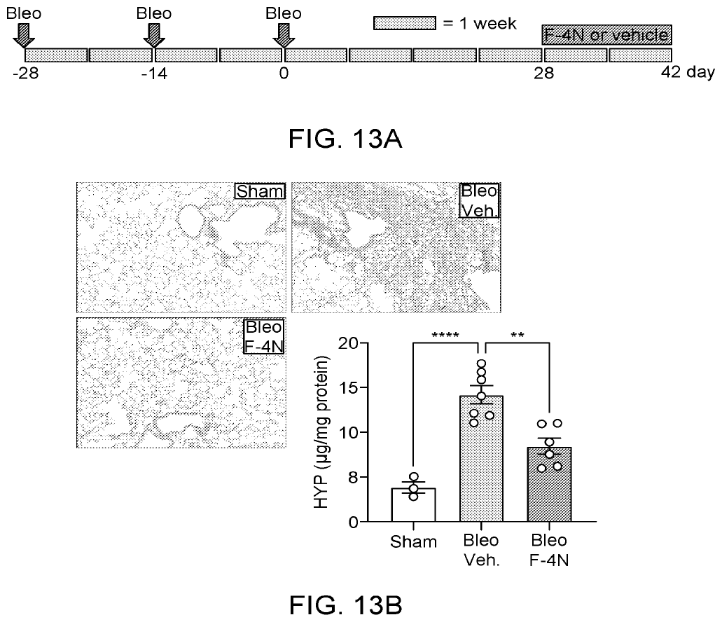

Mice received intratracheal bleomycin treatment 3× every 2 weeks. 28 days after the last bleomycin injury mice were treated with vehicle of F-4N daily for 14 days. A schematic of the study design is shown in FIG. 13A.

### Results

Efficacy of F-4N in a bleomycin injury model of lung fibrosis was evaluated.

Hydroxyproline analysis was performed on each lung from 8-week-old FVB wildtype mice administered bleomycin only (vehicle), bleomycin and F-4N, and sham mice. Representative H&E stained histological images of lungs are shown FIG. 13B.

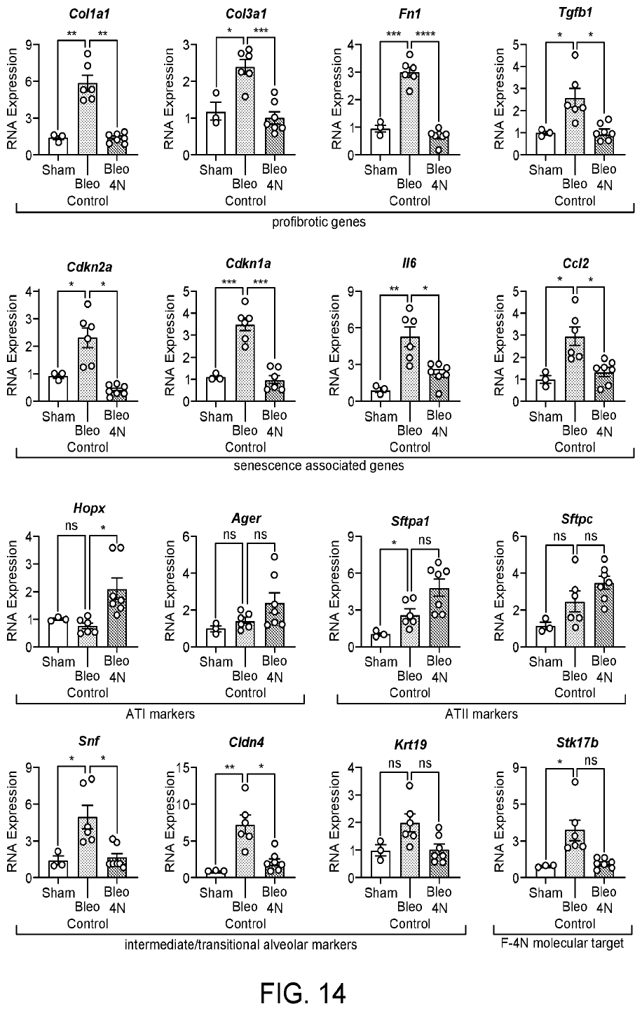

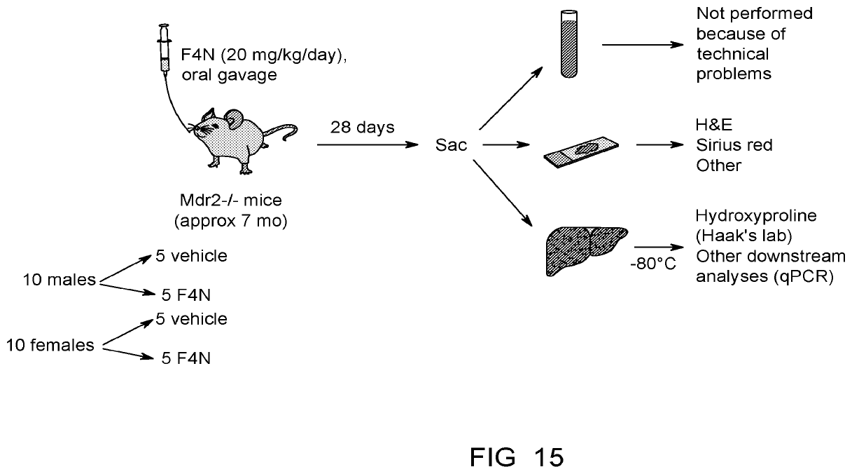

Whole lung RNA expression was also evaluated. Mouse lungs from FIG. 13B were analyzed by qPCR for changes in profibrotic genes, senescence associated genes, alveolar epithelial type I/type II markers, intermediate/transitional alveolar markers unique to fibrosis, and Stk17b (FIG. 14).

### Example 8: F-4N and PSC

The results in this Example re-present and expand on at least some of the results provided in other Examples.

### Methods

20 Mdr2^{-/-} mice (10 males, 10 females) approximately 7 months old with established liver fibrosis were assigned to receive either F-4N or vehicle (n=5 each group) for 4 weeks. At the end of treatment, mice were sacrificed, and livers were collected to assess liver inflammation and fibrosis. A schematic of the study design is shown in FIG. 15.

### Results

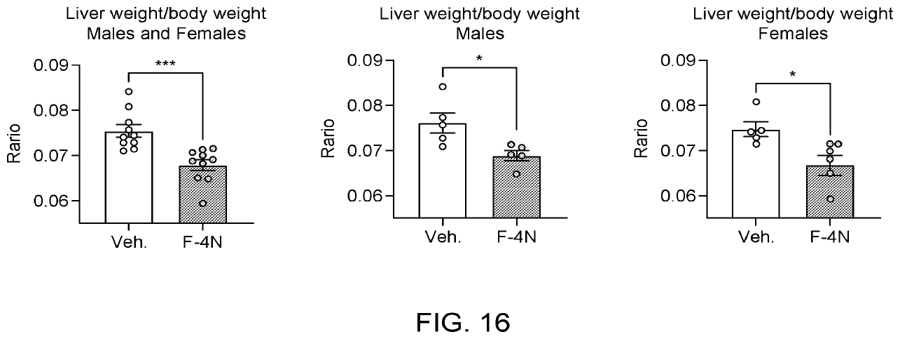

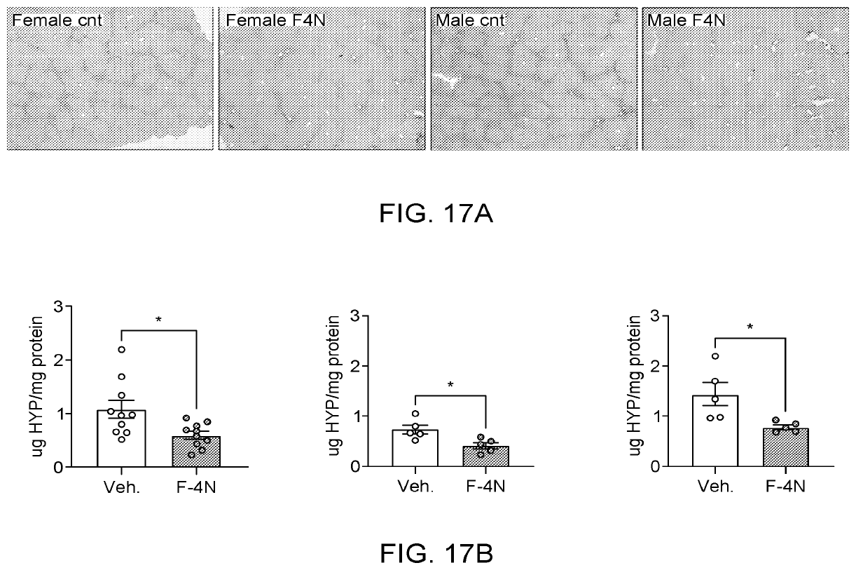

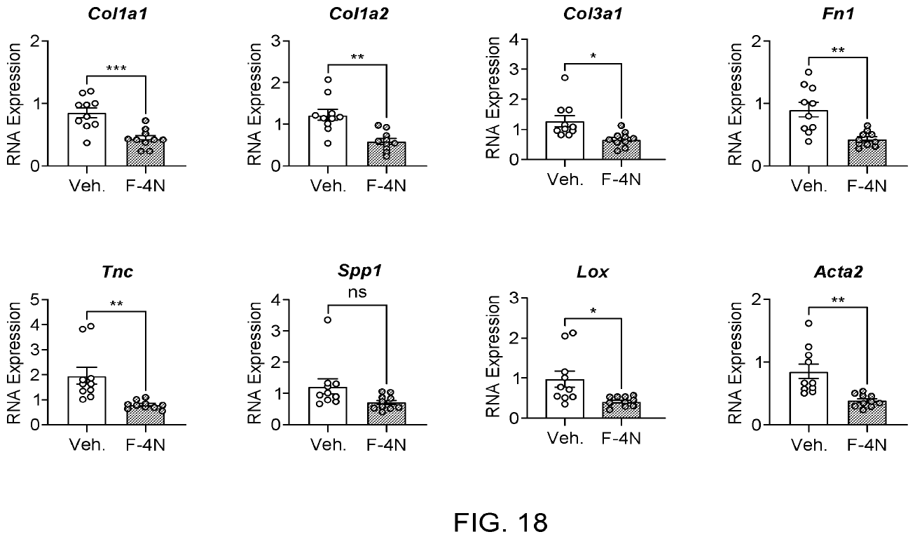

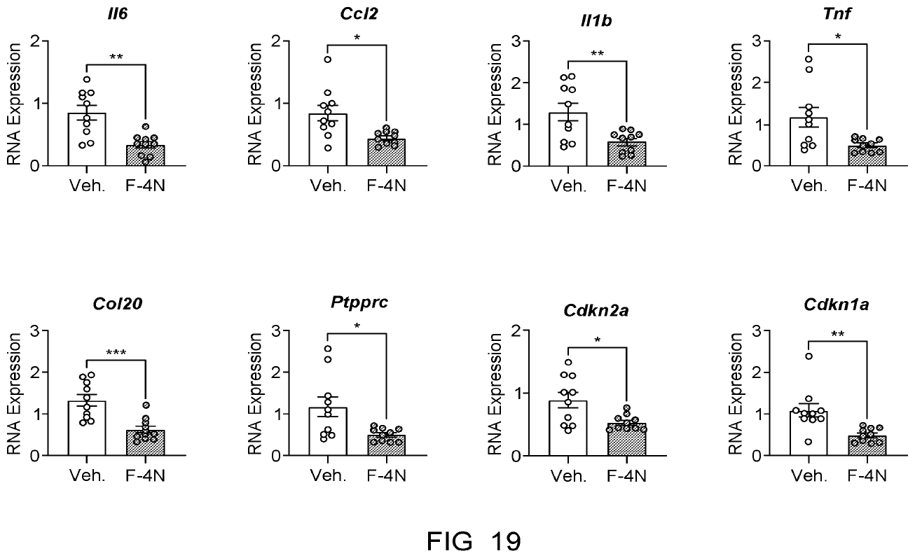

Oral delivery of F-4N for 4 weeks in a mouse model of PSC with established liver fibrosis resulted in:
Improvement of liver fibrosis, especially parenchymal fibrosis (“bridging fibrosis”), also confirmed by reduced expression of fibrogenic genes in the liver. See, FIGS. 16-18.
Significant reduction of tissue markers of liver inflammation and senescence. See, FIG. 19.

### Example 9: F-4N and Non-Alcoholic Steatohepatitis (NASH)

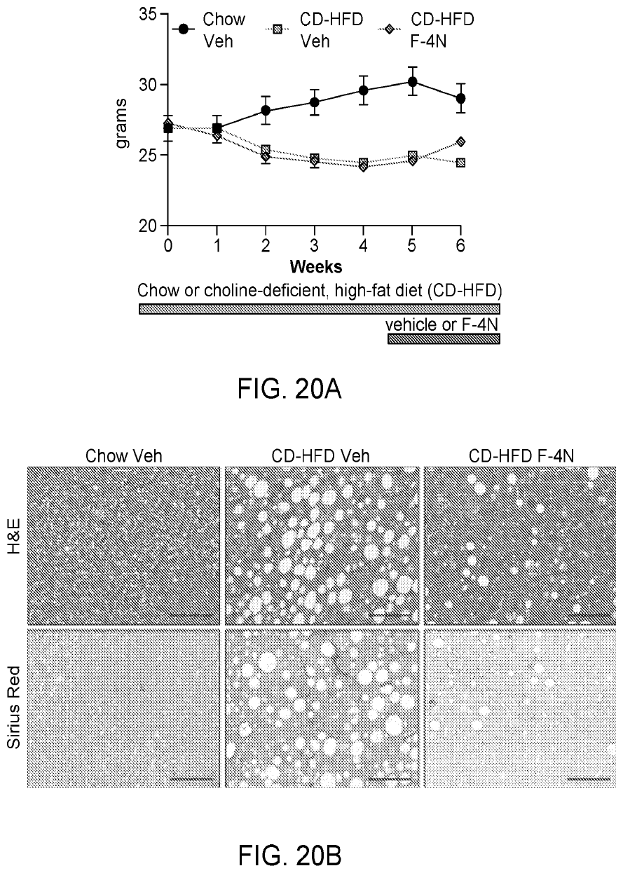

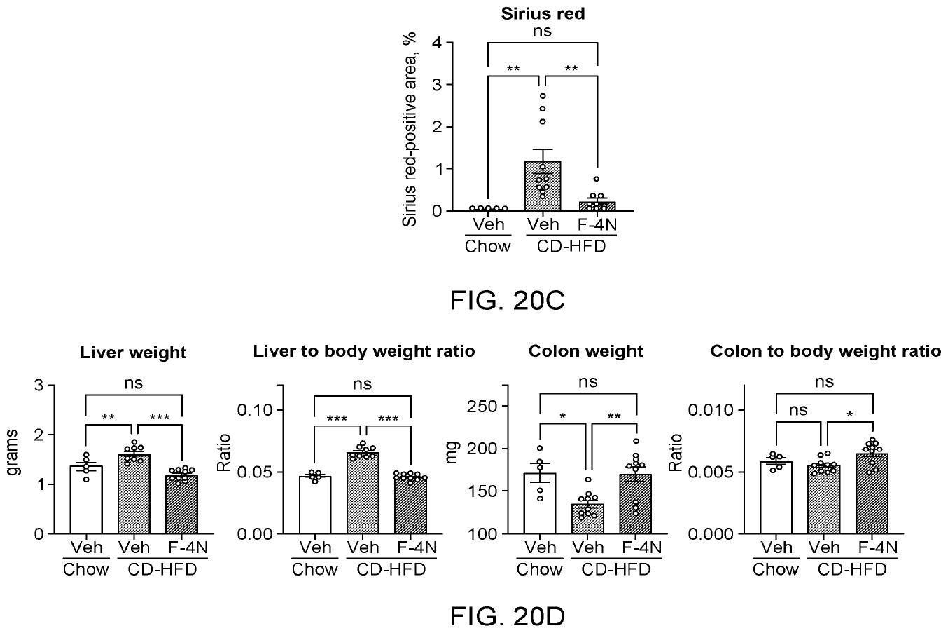

Mice in a mouse model of NASH were treated with 10 mg/kg F-4N daily i.p. for 2 weeks (FIG. 20A). Livers from treated mice were analyzed by H&E and Sirius Red staining (FIG. 20B). Sirius Red staining was quantified (FIG. 20C). Liver weight and colon weight changes were also assessed (FIG. 20D). These results suggested that F-4N was effective to treat NASH.

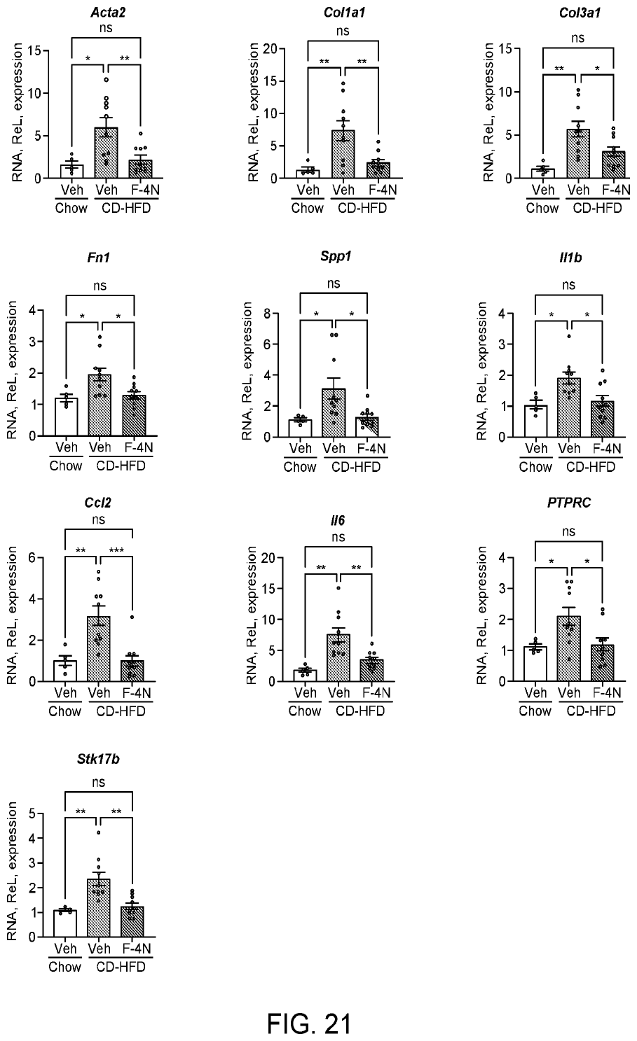

Livers from the study of FIG. 20 were analyzed by qPCR for expression of profibrotic genes, inflammatory genes, and for expression of Stk17b (the molecular target of F-4N). F-4N effectively reduced expression of profibrotic genes, inflammatory genes, and Stk17b (FIG. 21).

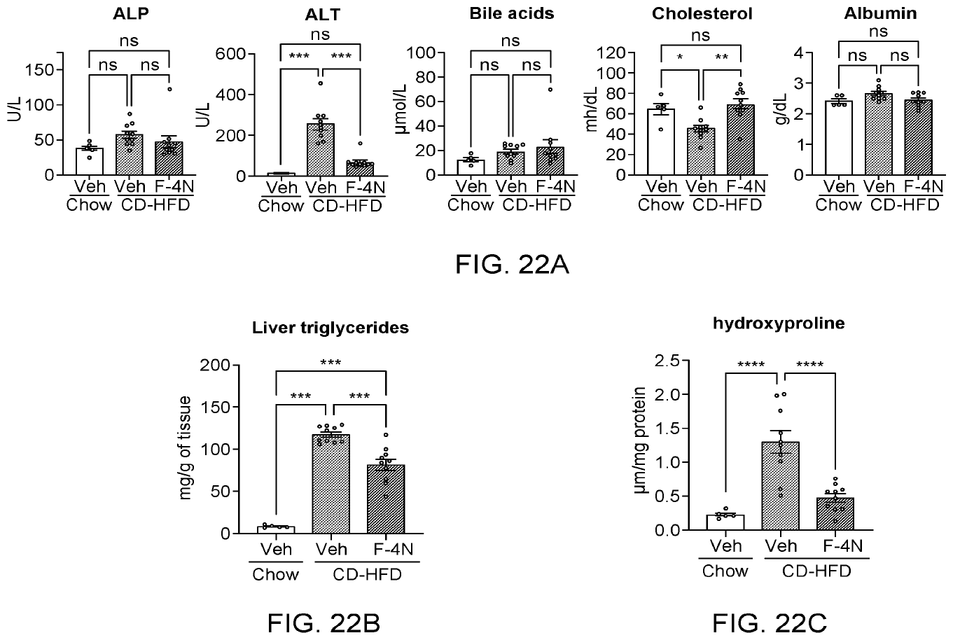

Further analysis of samples from the study of FIG. 20 were also performed. Liver function tests are shown in FIG. 22A, liver triglyceride analysis is shown in FIG. 22B, and hydroxyproline assessment of liver collagen content is shown in FIG. 22C. These results suggested that F-4N was effective to treat NASH.

### Example 10: F-4N and Ocular Fibrosis

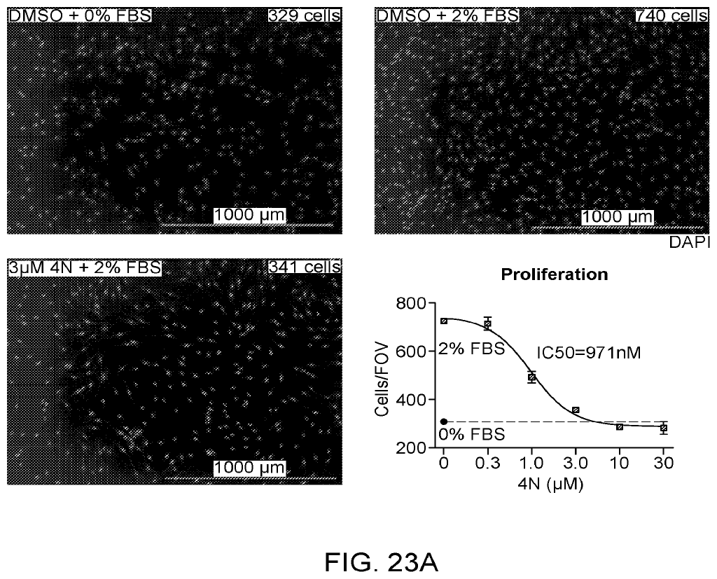

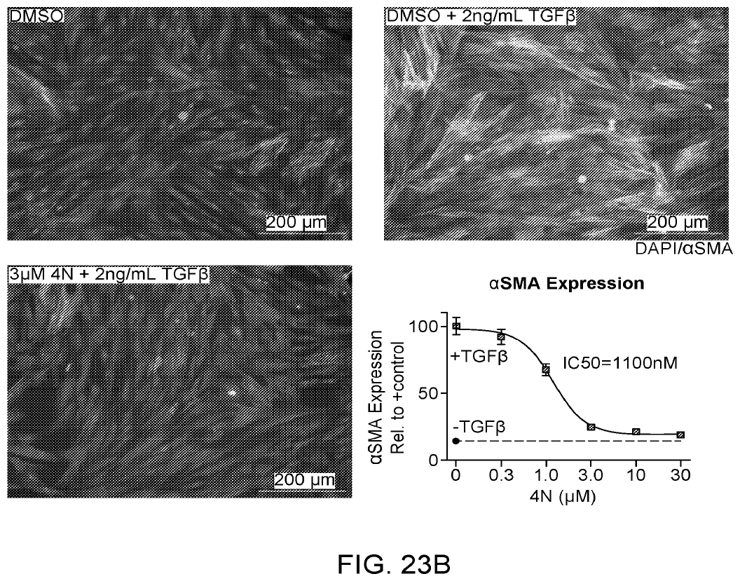

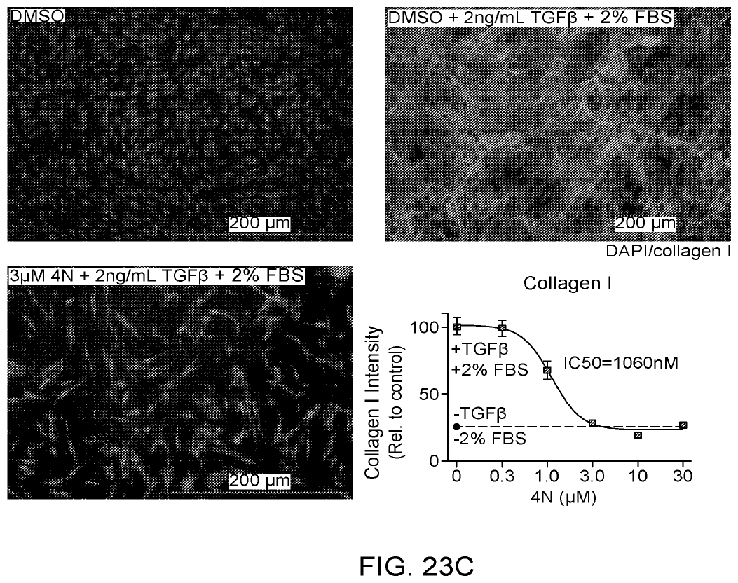

Conjunctival fibroblasts were cultured for 4 days in 2% FBS and with or without F-4N and evaluated for proliferation. Treated cells were fixed, stained with DAPI, and counted using automated software on a Cytation 5 (FIG. 23A).

Conjunctival fibroblasts were cultured for 3 days in 2% FBS with or without 2 ng/ml TGFβ and with or without F-4N and evaluated for the presence of fibroblasts. Treated cells were fixed, stained with DAPI, stained with an αSMA antibody, and quantified using automated software on a Cytation 5 (FIG. 23B).

Conjunctival fibroblasts cultured for 6 days in 2% FBS with or without 2 ng/ml TGFβ and with or without F-4N and evaluated for collagen deposition. Treated cells were fixed, stained with DAPI, stained with an antibody for collagen I, and quantified using automated software on a Cytation 5 (FIG. 23C).

Together, these results demonstrate that F-4N can be used to treat ocular fibrosis.

### Example 11: F-4N Pharmacology

### Dosing

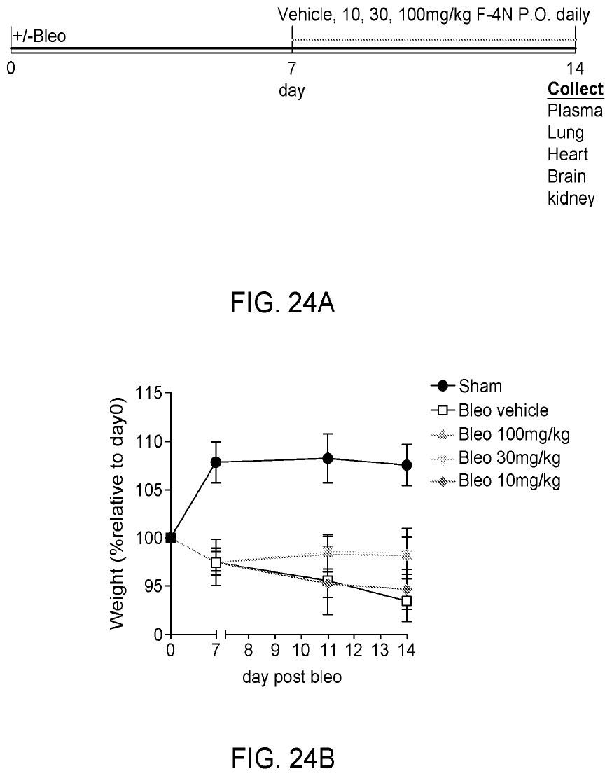

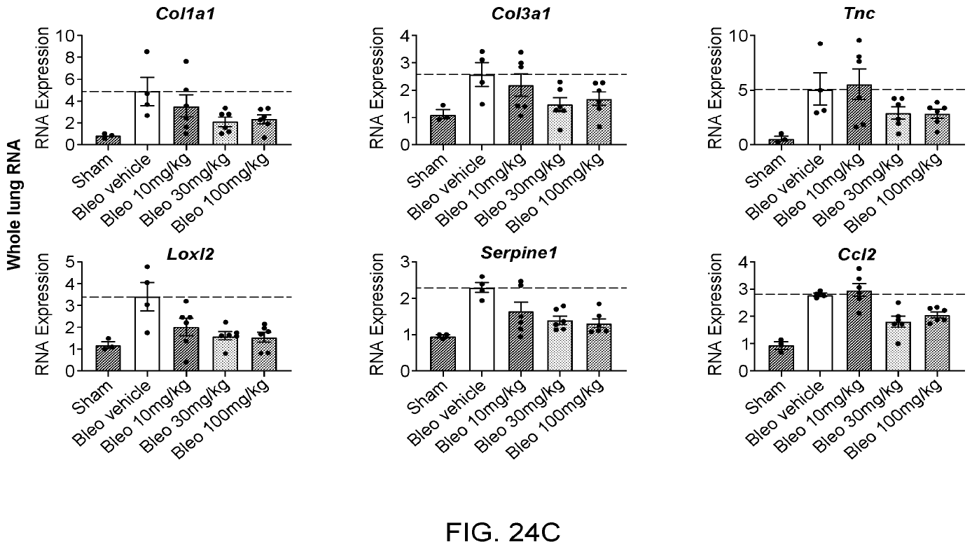

An acute exposure bleomycin study with varying doses of F-4N was performed as shown in FIG. 24A. On day 1 mice received intratracheal sham or bleomycin injury. On day 7 mice were divided into groups and treated daily with vehicle, 10, 30, or 100 mg/kg F-4N by oral gavage for 7 days. On day 14 organs and plasma were collected. Weight changes were measured throughout the experiment (FIG. 24B). Whole lung RNA expression of profibrotic genes as also examined (FIG. 24C). In this acute exposure, dose-finding study, 30 and 100 mg/kg daily oral gavage administration of F-4N improved weight change and reduced expression of profibrotic genes.

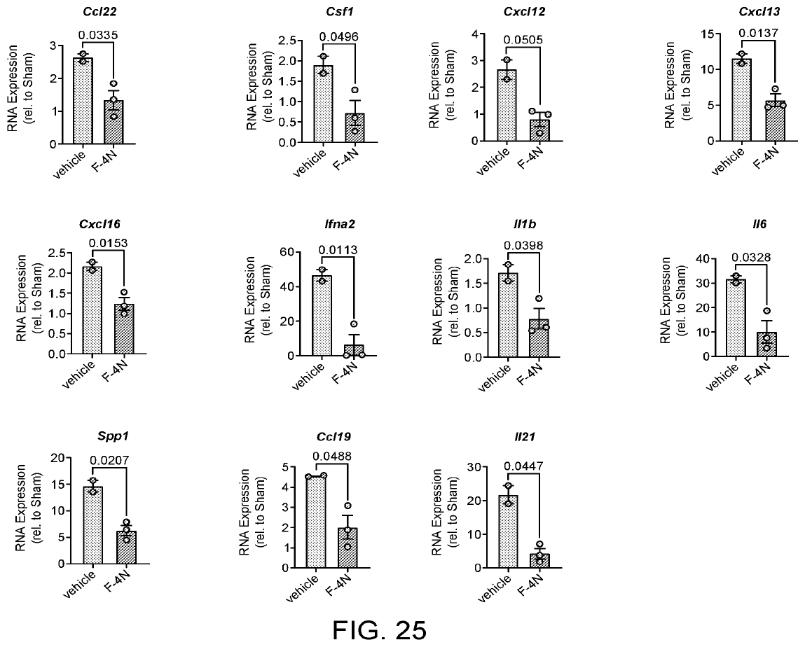

F-4N efficacy biomarkers were identified. RNA was collected from vehicle treated mice and from mice treated with 30 mg/kg F-4N. RNA was analyzed using the RT2 Profiler PCR array (Qiagen Catalog No.—330231) which measures expression of 89 different cytokines and chemokines. Eleven (11) genes were identified that exhibited reduced expression in mice after 7 days of treatment with F-4N (FIG. 25). A level of genes whose expression is altered in response to F-4N can be used as a biomarker for F-4N efficacy.

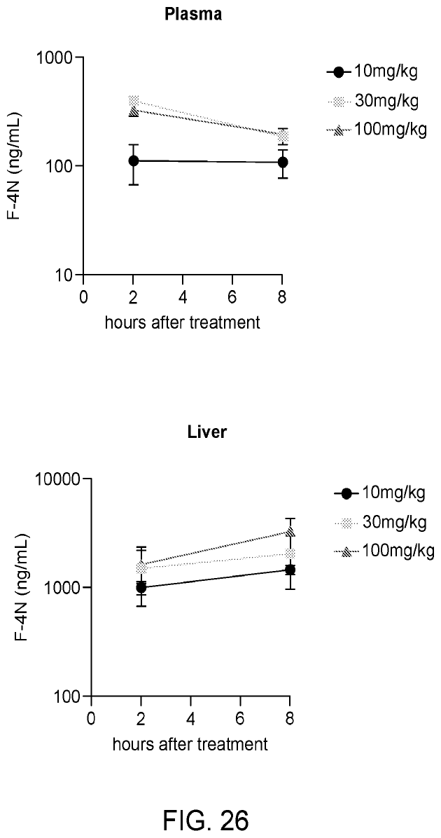

Levels of F-4N in the plasma and liver of mice following F-4N exposure were examined (FIG. 26). Plasma and liver tissue was collected 2 hours and 8 hours after the final dose from the mice subjected to the protocol described in FIG. 24A. Levels of unbound (free) F-4N were analyzed by Cyprotex (Framingham, MA) using LC-MS. Effective concentrations of F-4N were measured in both plasma and liver, with F-4N levels being higher in liver than plasma.

### Plasma Stability

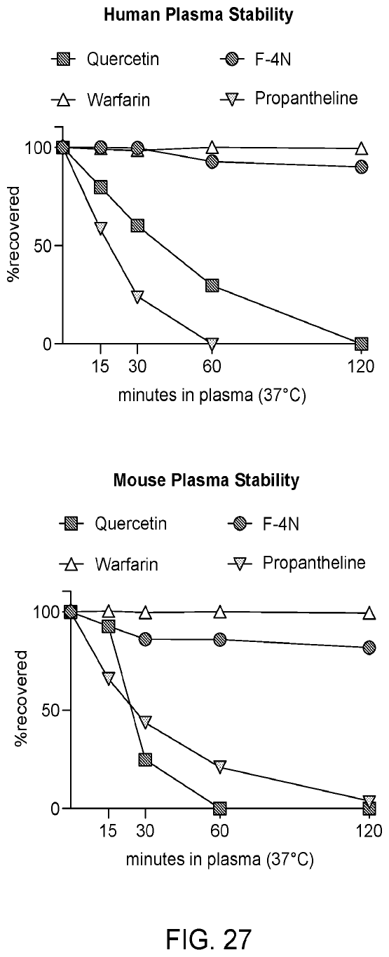

The stability of F-4N in plasma was assessed (FIG. 27). Compounds were incubated with human or mouse plasma. The percent of F-4N recovered was analyzed by LC-MS. Warfarin was used as a control for stable compound, and propantheline was used as a control for an unstable compound. Analysis was performed by Cyprotex (Framingham, MA). These results demonstrate that F-4N is stable in both human and mouse plasma.

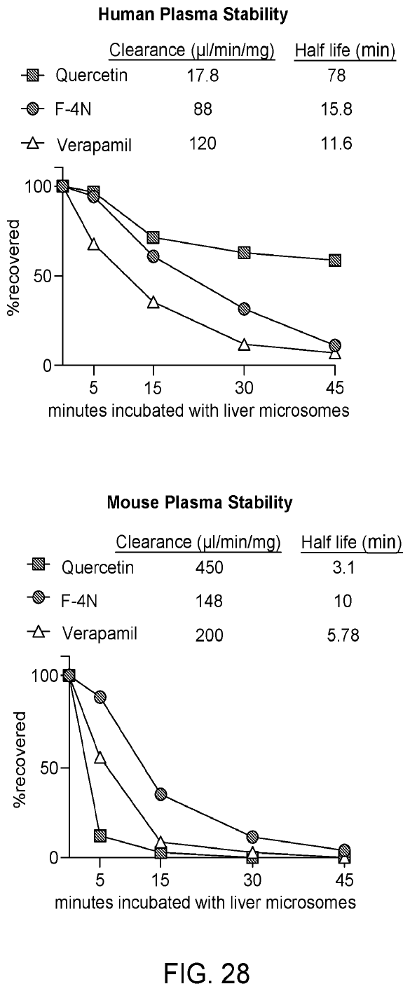

The microsomal stability of F-4N was also assessed (FIG. 28). Compounds were incubated with human or mouse liver derived microsomes. The percent of F-4N recovered was analyzed by LC-MS. Verapamil was used as a control for a rapidly degraded compound. Analysis was performed by Cyprotex (Framingham, MA). These results demonstrate that half-life and clearance rate of F-4N is in line with many clinically approved orally administered drugs.

Plasma protein binding assays were also performed (Table 2). Compounds were incubated with rat plasma for 4 hours. The percent of F-4N recovered was analyzed by LC-MS. Warfarin was used as a control for a compound with high plasma protein binding. Analysis performed by Cyprotex (Framingham, MA).

TABLE 2
Plasma protein binding assay.

| Test | Test | Test | Plasma | Plasma | Post-assay |
|---|---|---|---|---|---|
| article | Species | conc. | unbound | bound | recovery |
| F-4N | Rat | 5 μM | 0.94% | 99% | 100% |
| Quercetin | Rat | 5 μM | 5.0% | 95% | 0.80% |
| Warfarin | Rat | 2 μM | 0.971% | 99% | 100% |

These results demonstrate that F-4N displays high plasma protein binding.

### Example 12: Mechanism of F-4N

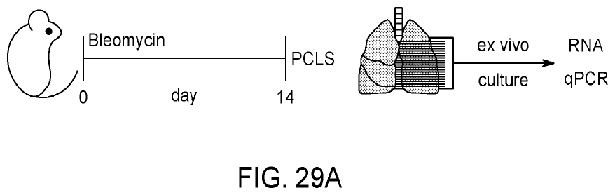

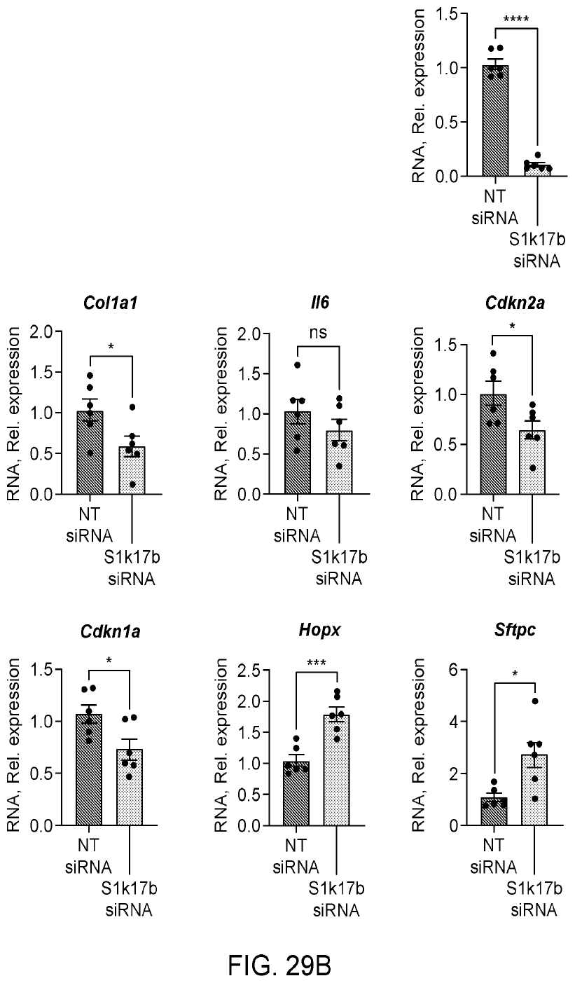

The effect of Stk17b on fibrosis markers was assessed. Stk17b was knocked down in a precision cut lung slice (PCLS) model. A schematic of the study protocol is shown in FIG. 29A. Six mice received intratracheal bleomycin on day 0. On day 14, during peak fibrosis, the lungs were harvested and sliced using a vibratome to generate 300 μM PCLSs. Tissues were cultured ex vivo for 4 days with either non-targeting siRNA or with siRNA targeting Stk17b. RNA was then collected and analyzed by qPCR (FIG. 29B). The results demonstrated that Stk17b can be targeted (e.g., can be reduced or inhibited) to treat lung fibrosis.

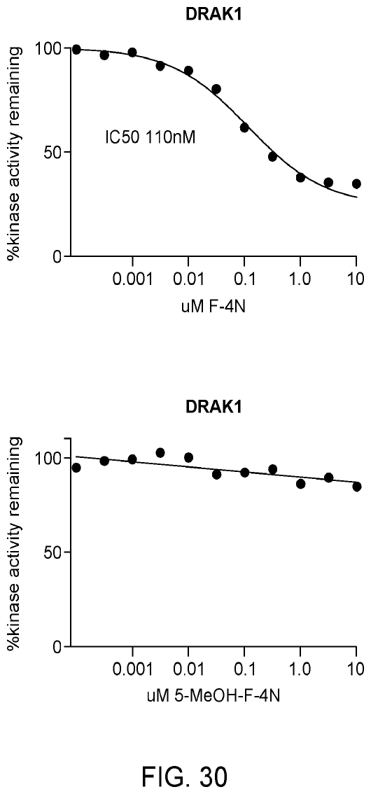

DRAK1 kinase activity was also assessed. Cells were incubated with DRAK1 and radiolabeled ATP and either F-4N or an inactive analog 5-MeOH-F-4N. A DRAK1 kinase activity was performed by Reaction Biology. DRAK1 kinase activity was measured and plotted (FIG. 30). These results demonstrated that F-4N can inhibit DRAK1 kinase activity. An analog inactive in phenotypic cell-based assays was inactive against DRAK1.

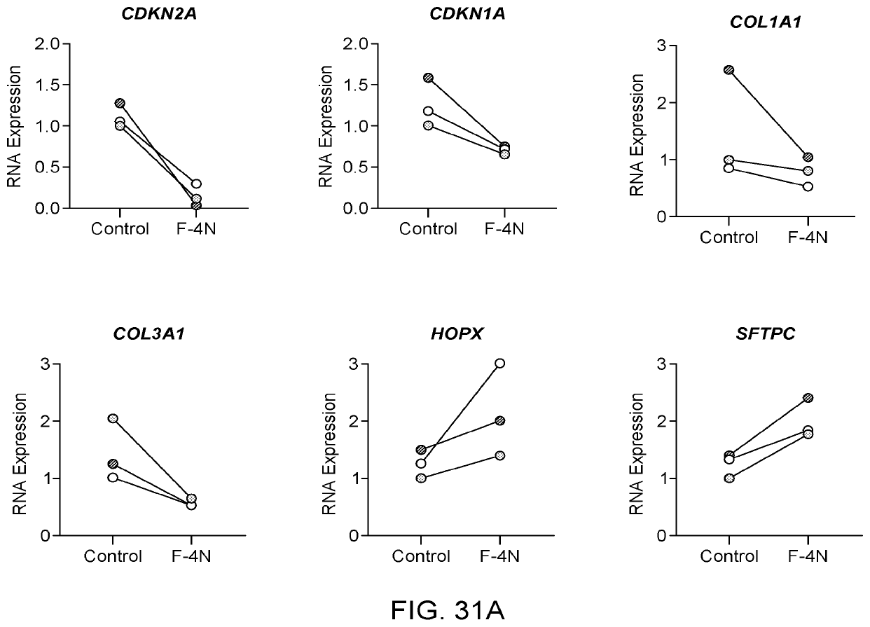

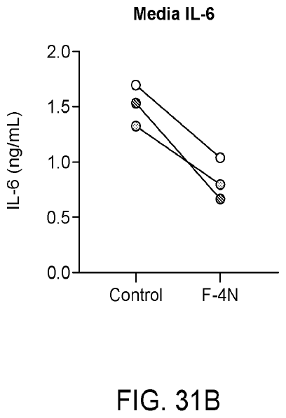

An IPF patient-derived organotypic ex vivo culture was used to determine efficacy of F-4N. Sections of lung tissue (about 500 μM) were cut from IPF patient transplant lungs and cultured for 4 days ex vivo with DMSO (0.1%) or F-4N (3 μM). Lung tissue slices were collected, RNA was isolated, and qPCR analysis was performed (FIG. 31). Media was also collected for ELISA analysis for IL-6 (FIG. 31B). N=3 patient samples. These results demonstrated that F-4N reduced expression of fibrotic markers and enhanced expression of mature alveolar epithelial cell markers.

### Example 13: Structural Optimization of Quercetin to Enhance Pharmacological Properties

This Example describes the identification of quercetin structural that can enhance senolytic capacity and can enhance transdifferentiation blockade in pulmonary fibroblasts.

### Comparative Flavonoid Efficacy and Lack of Toxicity in Aged Brain and Liver.

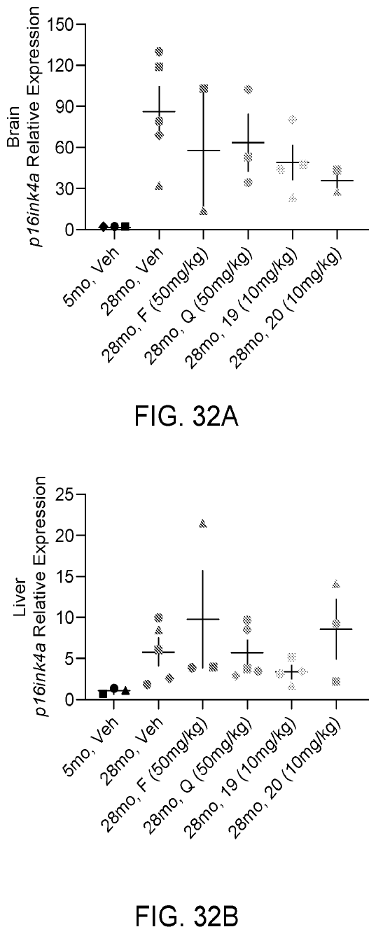

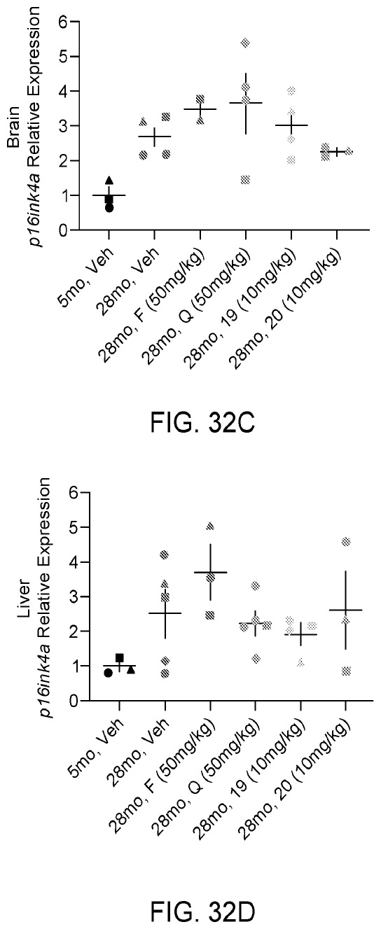

Five-month-old and twenty-eight month old wild type mice were oral gavage treated with the indicated concentrations of flavonoids (F, Q, compound 19, or compound 20) or vehicle for four consecutive days and were euthanized one week later. Real-time PCR analysis suggests that low dose administration of compound 19 and/or 20 may reduce expression of a key senescence-activation gene, p16ink4a, in (FIG. 32A) brain and (FIG. 32B) liver more robustly than fisetin (F) or quercetin (Q), which have established senolytic effects at comparatively higher doses. Analysis of CD68 expression, an indicator of inflammatory activation, demonstrates a lack of drug-induced toxicity in brain (FIG. 32C) or liver (FIG. 32D).

### Quercetin Analogs can Potently Kill Senescent Fibroblasts

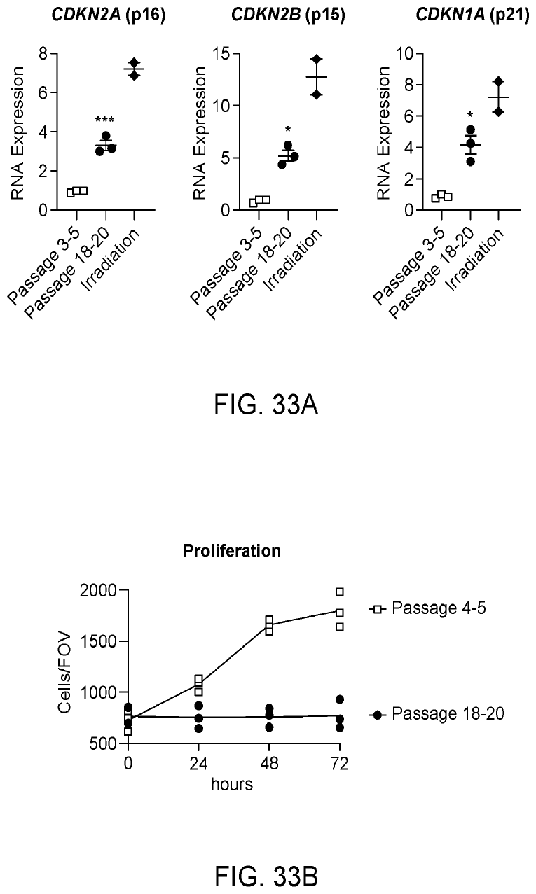

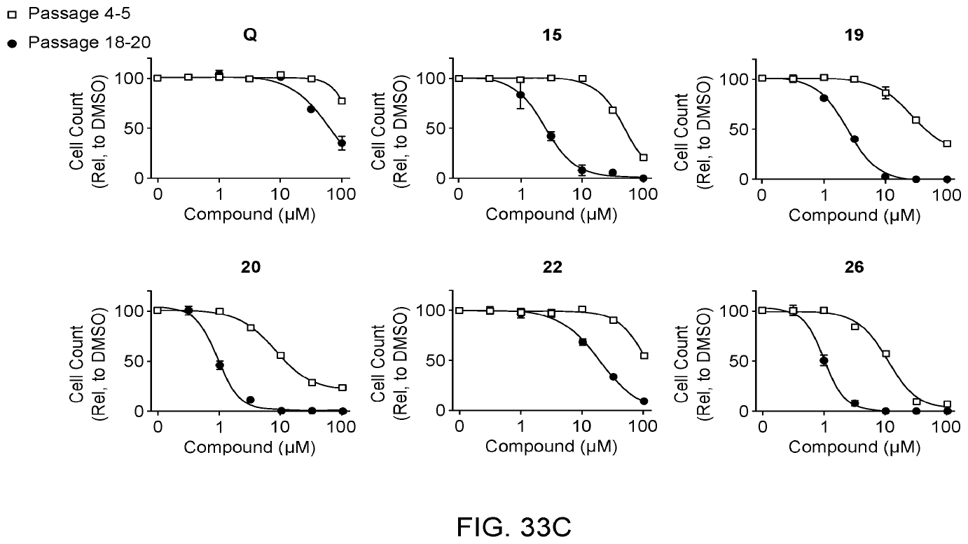

Expression of senescence markers (FIG. 33A) and proliferation (FIG. 33B) were measured in induced senescent fibroblasts treated with quercetin analogs. Over 30 diverse quercetin analogs were screened for their capacity to kill senescent cells more potently than low passage proliferative fibroblasts. Shown in FIG. 33C are the most potent derivatives, several exhibiting nanomolar to low micromolar potency.

### TGFβ and Senescent Cell Conditioned Media can Promote Fibroblast to Myofibroblast Transdifferentiation

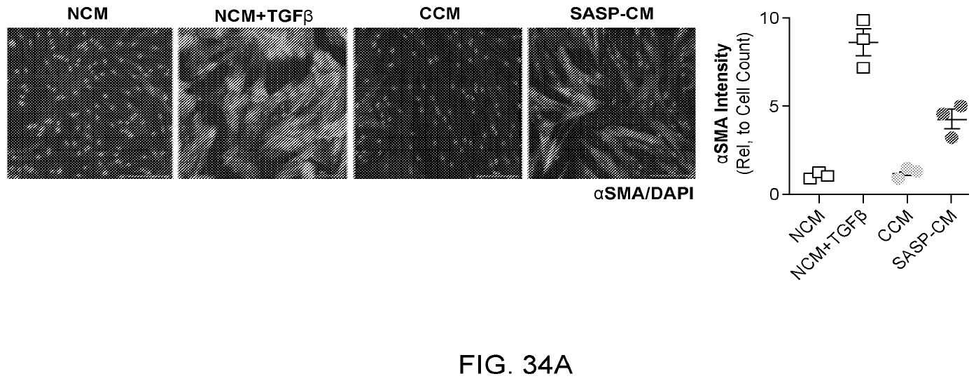

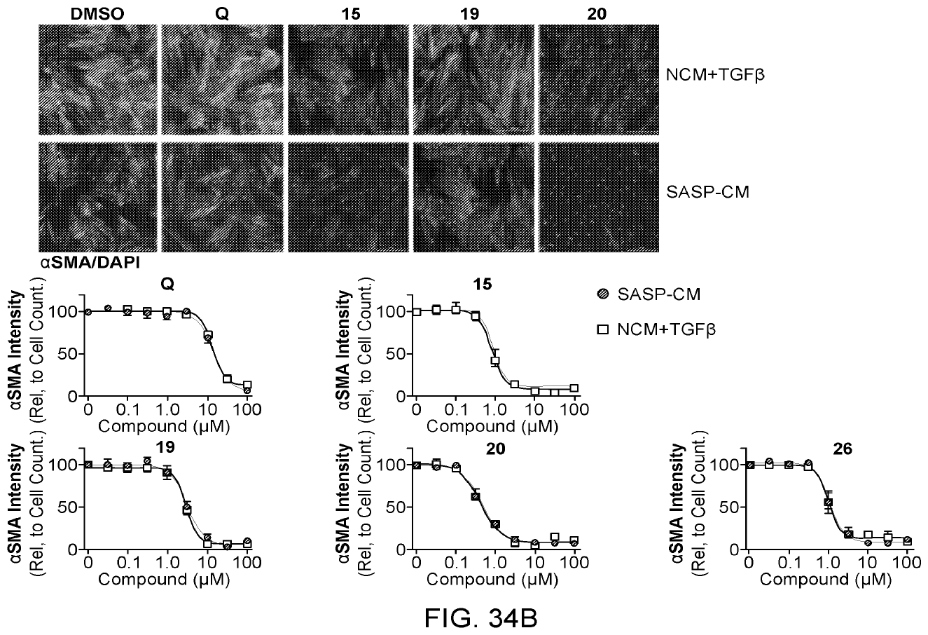

Fibroblasts were detected by αSMA staining and staining intensity was quantified (FIG. 34A). Markers of fibroblast activation were also assessed (FIG. 34B). These results demonstrated that quercetin analogs can potently prevent fibroblast activation.

### Quercetin Analogs can Prevent SASP-CM and TGFβ Induced Collagen Deposition.

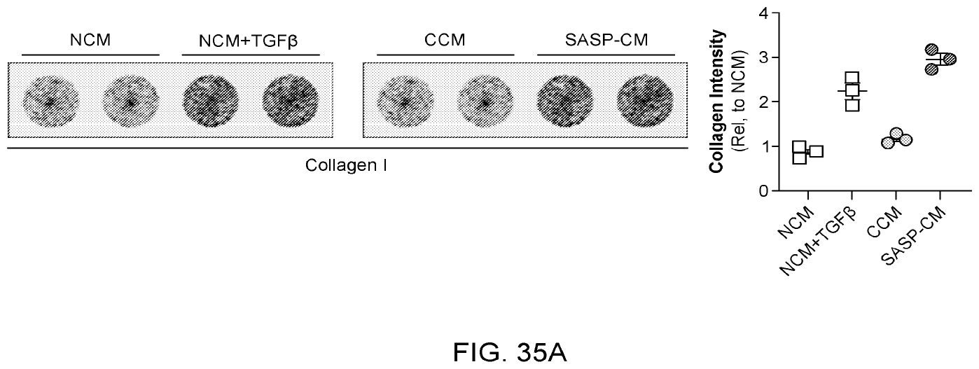

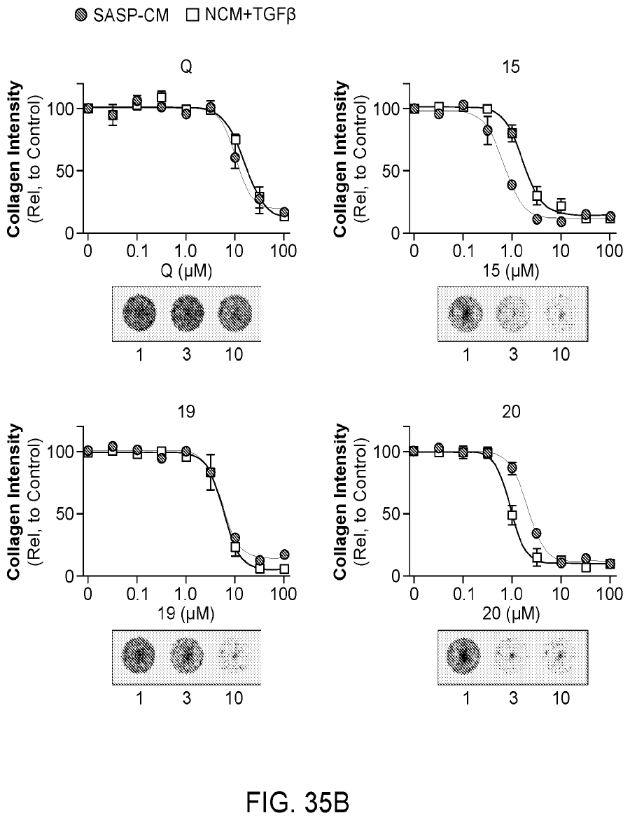

TGFβ and senescent cell conditioned media promote collagen I deposition (FIG. 35A). Quercetin analogs potently prevent collagen I deposition (FIG. 35B).

### Quercetin Analogs can Prevent TGFβ Induced Profibrotic Gene Expression

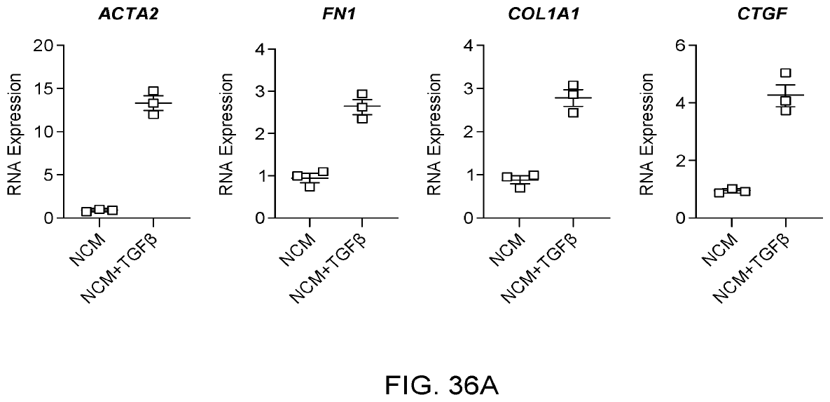

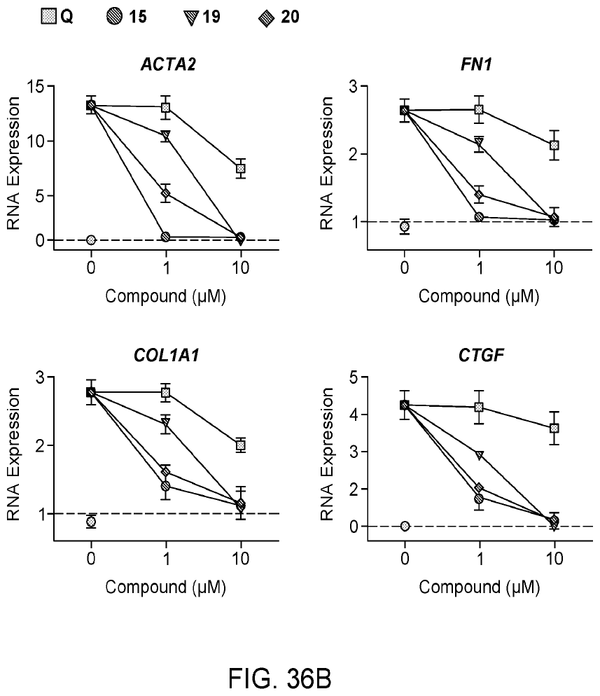

TGFβ promotes profibrotic gene expression (FIG. 36A). Quercetin analogs potently block profibrotic gene expression (FIG. 36B).

Quercetin Analogs Having a p-Ethoxy have Enhanced Activity

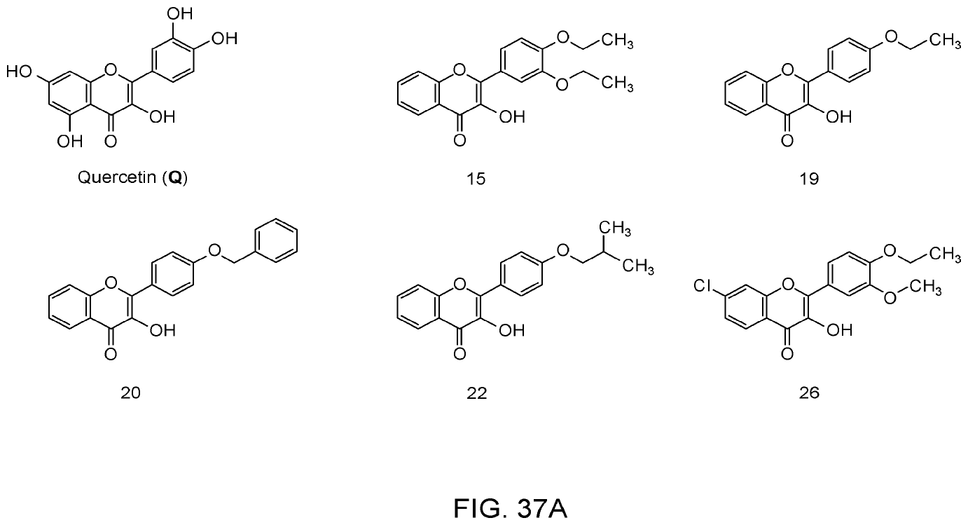

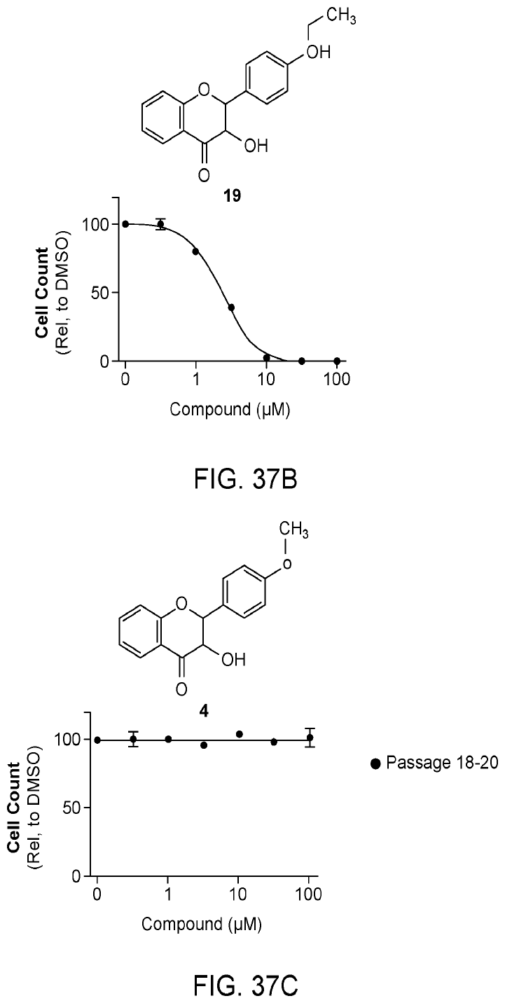

Exemplary quercetin analogs having a p-ethoxy are shown in FIG. 37A. A graph of cell proliferation showing that a quercetin analog having a p-ethoxy induced cellular senescence (FIG. 37B). A graph of cell proliferation showing that a quercetin analog lacking a p-ethoxy did not induce cellular senescence (FIG. 37C).

### Example 14: Treating IPF

A human identified as having IPF is administered or self-administers a composition including one or more flavonoid compounds having the structure of Formula (II). In some cases, the administered flavonoid compound(s) can reduce the severity of one or more symptoms of IPF. In some cases, the administered flavonoid compound(s) can reduce the amount of fibrotic scarring in the lung(s) of the human.

### Example 15: Treating PSC

A human identified as having PSC is administered or self-administers a composition including one or more flavonoid compounds having the structure of Formula (II). In some cases, the administered flavonoid compound(s) can reduce the severity of one or more symptoms of PSC. In some cases, the administered flavonoid compound(s) can reduce the amount of fibrotic scarring in the liver of the human.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising a flavonoid compound having a structure of Formula (I): 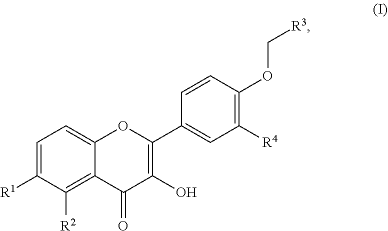
or a pharmaceutically acceptable salt thereof, wherein:
R¹ is selected from the group consisting of H, OH, a C₁-C₄ alkyl, a halogen, and a C₁-C₄ alkoxy;
R² is selected from the group consisting of H, OH, a C₁-C₄ alkyl, a halogen, and a C₁-C₄ alkoxy;
R³ is selected from the group consisting of H, CH₂CH₃, cyclopropyl, phenyl, 2-pyridinyl, 3-pyridinyl, and 4-pyridinyl;
and R⁴ is selected from the group consisting of H, OH, a C₁-C₄ alkyl, a halogen, and a C₁-C₄ alkoxy.

2. The composition of claim 1, wherein said flavonoid compound of Formula (I) has structure:

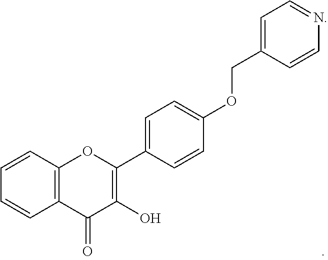 [SMILES: O=c1c(O)c(-c2ccc(OCc3ccncc3)cc2)oc2ccccc12]

3. The composition of claim 1, wherein said flavonoid compound of Formula (I) has structure:

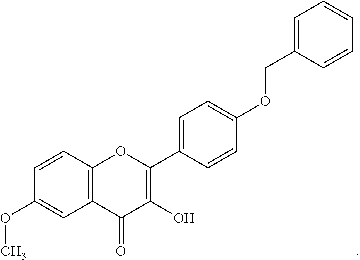 [SMILES: COc1ccc2oc(-c3ccc(OCc4ccccc4)cc3)c(O)c(=O)c2c1]

4. The composition of claim 1, wherein said flavonoid compound of Formula (I) has structure:

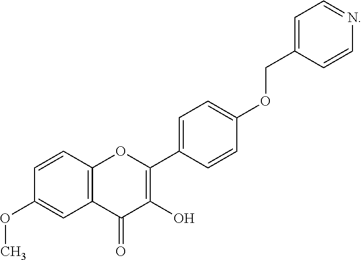 [SMILES: COc1ccc2oc(-c3ccc(OCc4ccncc4)cc3)c(O)c(=O)c2c1]

5. The composition of claim 1, said composition further comprising a pharmaceutically acceptable carrier, excipient, or diluent.

6. (canceled)

7. A method for (a) treating a mammal having a fibrotic condition, wherein said method comprises administering a composition of to said mammal, (b) reducing fibrosis in a mammal having a fibrotic condition, wherein said method comprises administering the composition to said mammal, (c) reducing a number of senescent cells in a mammal having a fibrotic condition, wherein said method comprises administering the composition to said mammal, or (d) inhibiting a serine/threonine kinase 17 (STK17) polypeptide in a mammal, wherein said method comprises administering the composition to said mammal,
wherein the composition comprises a flavonoid compound having a structure of Formula (I): 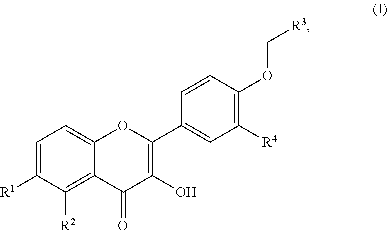
or a pharmaceutically acceptable salt thereof, wherein:
R¹ is selected from the group consisting of H, OH, a C₁-C₄ alkyl, a halogen, and a C₁-C₄ alkoxy;
R² is selected from the group consisting of H, OH, a C₁-C₄ alkyl, a halogen, and a C₁-C₄ alkoxy;
R³ is selected from the group consisting of H, CH₂CH₃, cyclopropyl, phenyl, 2-pyridinyl, 3-pyridinyl, and 4-pyridinyl;
and R⁴ is selected from the group consisting of H, OH, a C₁-C₄ alkyl, a halogen, and a C₁-C₄ alkoxy.

8. The method of claim 7, wherein said mammal is a human.

9. The method of claim 7, wherein said method comprises (a).

10. The method of claim 7, wherein said method comprises (b).

11. The method of claim 7, wherein said method comprises (c).

12. The method of claim 7, wherein said method comprises (d).

13-31. (canceled)

32. A composition comprising a flavonoid compound having a structure of Formula (II): 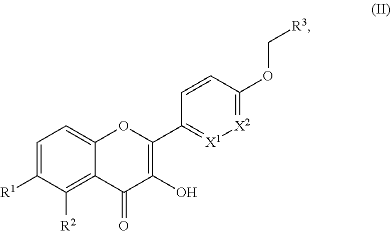
or a pharmaceutically acceptable salt thereof, wherein:
X¹ is selected from N and CH;
X² is selected from N and CR⁴;
R¹ is selected from the group consisting of H, OH, a C₁-C₄ alkyl, a halogen, and a C₁-C₄ alkoxy;
R² is selected from the group consisting of H, OH, a C₁-C₄ alkyl, a halogen, and a C₁-C₄ alkoxy;
R³ is selected from the group consisting of H, CH₃, CH₂CH₃, cyclopropyl, phenyl, 4-OH-phenyl, 2-OH-phenyl, 3-OH-phenyl, 2-pyridinyl, 3-pyridinyl, 4-pyridinyl, thiophen-2-yl, thiophen-3-yl, tetrahydrofuran-2-yl, and tetrahydrofuran-3-yl;
and R⁴ is selected from the group consisting of H, OH, a C₁-C₄ alkyl, a halogen, and a C₁-C₄ alkoxy.

33. The composition of claim 32, wherein said flavonoid compound of Formula (II) has any one of the following formulae: 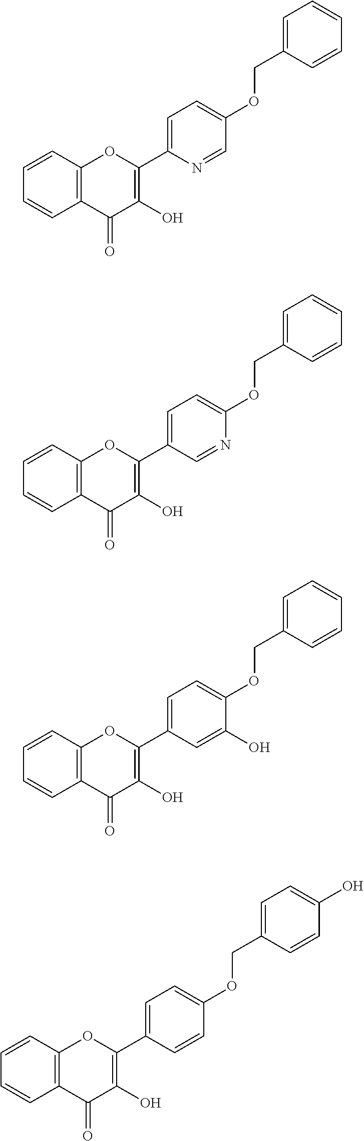 [SMILES: O=c1c(O)c(-c2ccc(OCc3ccccc3)cn2)oc2ccccc12 ; O=c1c(O)c(-c2ccc(OCc3ccccc3)nc2)oc2ccccc12 ; O=c1c(O)c(-c2ccc(OCc3ccccc3)c(O)c2)oc2ccccc12 ; O=c1c(O)c(-c2ccc(OCc3ccc(O)cc3)cc2)oc2ccccc12] 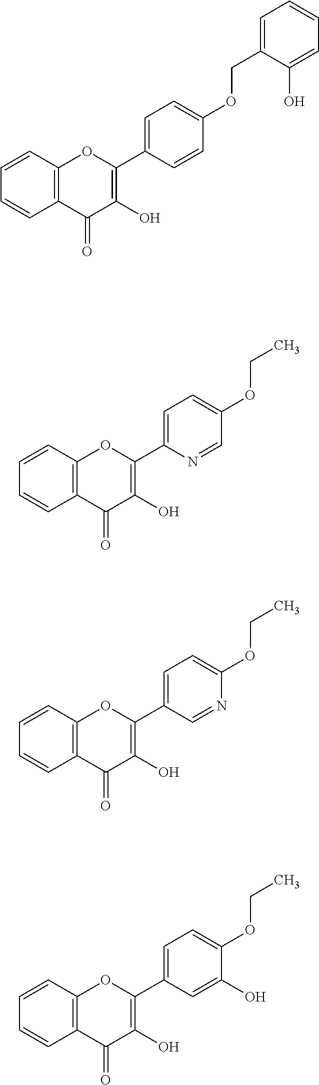 [SMILES: O=c1c(O)c(-c2ccc(OCc3ccccc3O)cc2)oc2ccccc12 ; CCOc1ccc(-c2oc3ccccc3c(=O)c2O)nc1 ; CCOc1ccc(-c2oc3ccccc3c(=O)c2O)cn1 ; CCOc1ccc(-c2oc3ccccc3c(=O)c2O)cc1O] 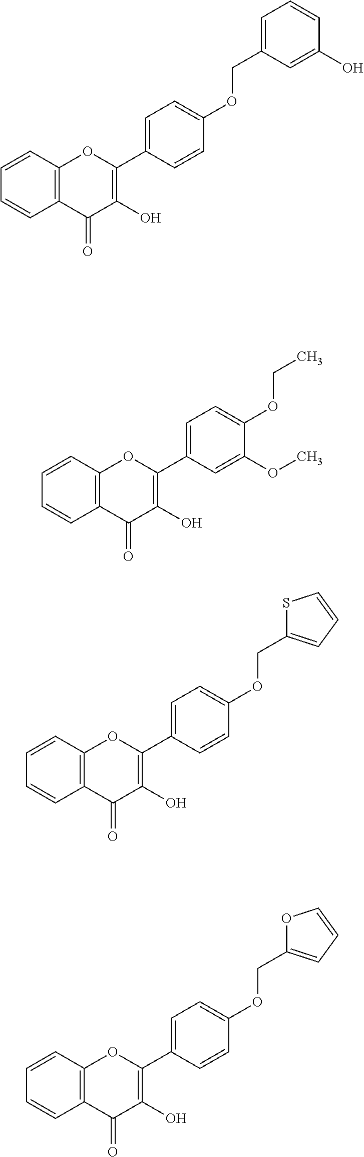 [SMILES: O=c1c(O)c(-c2ccc(OCc3cccc(O)c3)cc2)oc2ccccc12 ; CCOc1ccc(-c2oc3ccccc3c(=O)c2O)cc1OC ; O=c1c(O)c(-c2ccc(OCc3cccs3)cc2)oc2ccccc12 ; O=c1c(O)c(-c2ccc(OCc3ccco3)cc2)oc2ccccc12] 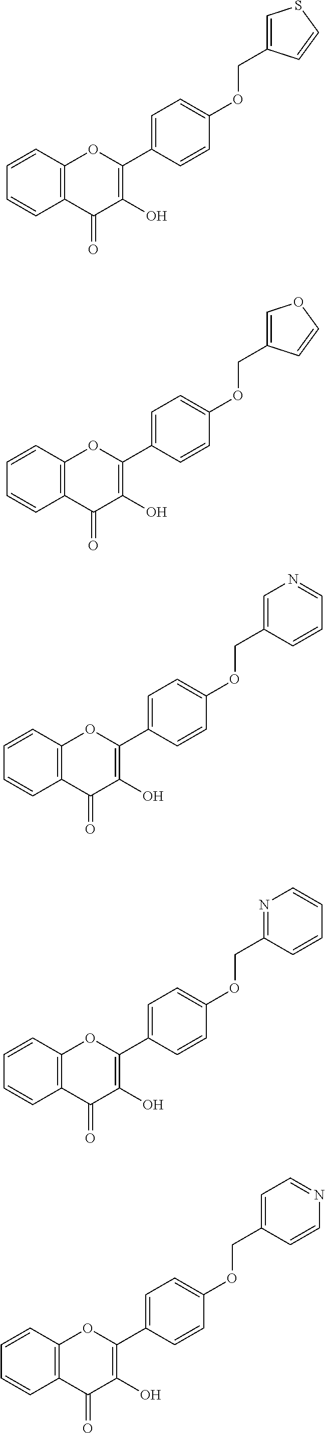 [SMILES: O=c1c(O)c(-c2ccc(OCc3ccsc3)cc2)oc2ccccc12 ; O=c1c(O)c(-c2ccc(OCc3ccoc3)cc2)oc2ccccc12 ; O=c1c(O)c(-c2ccc(OCc3cccnc3)cc2)oc2ccccc12 ; O=c1c(O)c(-c2ccc(OCc3ccccn3)cc2)oc2ccccc12 ; O=c1c(O)c(-c2ccc(OCc3ccncc3)cc2)oc2ccccc12]
or a pharmaceutically acceptable salt thereof.

34. (canceled)

35. A method for (a) treating a mammal having a fibrotic condition, wherein said method comprises administering a composition to said mammal, (b) reducing fibrosis in a mammal having a fibrotic condition, wherein said method comprises administering the composition to said mammal, (c) reducing a number of senescent cells in a mammal having a fibrotic condition, wherein said method comprises administering the composition to said mammal, or (d) inhibiting a STK17 polypeptide in a mammal, wherein said method comprises administering the composition to said mammal,
wherein the composition comprises a flavonoid compound having a structure of Formula (II): 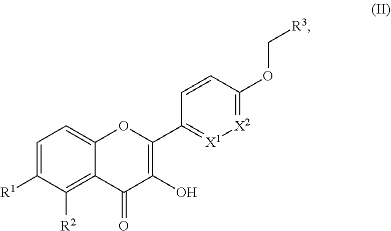
or a pharmaceutically acceptable salt thereof, wherein:
X¹ is selected from N and CH;
X² is selected from N and CR⁴;
R¹ is selected from the group consisting of H, OH, a C₁-C₄ alkyl, a halogen, and a C₁-C₄ alkoxy;
R² is selected from the group consisting of H, OH, a C₁-C₄ alkyl, a halogen, and a C₁-C₄ alkoxy;
R³ is selected from the group consisting of H, CH₃, CH₂CH₃, cyclopropyl, phenyl, 4-OH-phenyl, 2-OH-phenyl, 3-OH-phenyl, 2-pyridinyl, 3-pyridinyl, 4-pyridinyl, thiophen-2-yl, thiophen-3-yl, tetrahydrofuran-2-yl, and tetrahydrofuran-3-yl;
and R⁴ is selected from the group consisting of H, OH, a C₁-C₄ alkyl, a halogen, and a C₁-C₄ alkoxy.

36. The method of claim 35, wherein said mammal is a human.

37. The method of claim 35, wherein said method comprises (a).

38. The method of claim 35, wherein said method comprises (b).

39. The method of claim 35, wherein said method comprises (c).

40. The method of claim 35, wherein said method comprises (d).

41-57. (canceled)
